(19) United States
(12) Patent Application Publication
(10) Pub. No.: US 2024/0016853 A1
(43) Pub. Date: Jan. 18, 2024

(54) MODIFIED IPSCS

(71) Applicant: ADAPTIMMUNE LIMITED, Abingdon, Oxfordshire (GB)

(72) Inventors: Garth HAMILTON, Abingdon, Oxfordshire (GB); Christine SEIDL, Abingdon, Oxfordshire (GB)

(21) Appl. No.: 17/924,926

(22) PCT Filed: May 11, 2021
(86) PCT No.: PCT/GB2021/051125

§ 371 Date: Mar. 17, 2023

(30) Foreign Application Priority Data
May 11, 2020 (GB) 2006903.5

(51) Int. Cl. A61K 35/545; C12N 9/16; A61K 35/28; A61K 39/00; C07K 14/725; A61P 35/00

(52) U.S. Cl. CPC A61K 35/545; C12N 9/16; C12Y 301/03048; A61K 35/28; A61K 39/4611; C07K 14/7051; A61P 35/00

(57) ABSTRACT

The present invention provides a modified induced pluripotent stem cell iPSC or haemogenic lineage cell comprising at least one heterologous nucleic acid sequence encoding a heterologous T-cell receptor (TCR) integrated in the cell genome and uses thereof.

### INTRODUCTION

The present invention provides a modified induced pluripotent stem cell iPSC or haemogenic lineage cell comprising at least one heterologous nucleic acid sequence encoding a heterologous T-cell receptor (TCR) integrated in the cell genome and uses thereof.

### BACKGROUND

Adoptive T-cell therapy is widely recognised as an important therapeutic intervention for the treatment of cancer. Most current approaches use autologous or patient-derived T-cells. These include TILs (tumour infiltrating lymphocytes) or T-cells that have been virally transduced to express a CAR (chimeric antigen receptor) or affinity enhanced TCR (T-cell receptor).

One drawback of autologous therapies is the complexity associated with their manufacture. An alternative “off-the-shelf” approach is the production of engineered T-cells from an allogeneic source, such healthy donor derived T-cells or T-cells that have been differentiated from a human induced pluripotent stem cell (hiPSC). Additionally patients must wait for an apheresis slot to collect their own lymphocytes and the manufacturing time for the T cell product to be made, additionally patients may also suffer T cell dysfunction or have had multiple lines of prior chemotherapy, impairing their own lymphocyte count and function such that the cells available for manufacture are low in count or functionality at the outset.

The allogeneic approach is particularly advantageous as it may provide a more readily available supply of a uniform cell product that can be could be screened for a T cell phenotype linked with more a more functional and potent, anti-cancer capability associated with improved response. In short patients will get treatment faster with a more defined T-cell phenotype that does not vary from patient to patient. It is expected that gene editing can provide quick access to T cell therapy at reduced cost for a standardised high quality T cell product, for a much wider group of patients who have the most aggressive and resistant disease

The present invention provides allogeneic hiPSC derived T-cells (iT-cells) expressing a recombinant heterologous T-cell receptor (TCR) of defined tumour antigen specificity, a SPEAR TCR (Specific Peptide Enhanced Affinity Receptor).

A prerequisite for any allogeneic T-cell product which uses an αβTCR is the production of a clonal T-cell population. This is required to mitigate the risk of graft versus host disease (GvHD). iT-cells with restricted expression of a defined αβTCR have been successfully produced from clones of hiPSC that were transduced with lentiviral vectors encoding a specific TCR [Minagawa, A., et al., Enhancing T Cell Receptor Stability in Rejuvenated iPSC-Derived T Cells Improves Their Use in Cancer Immunotherapy. Cell Stem Cell, 2018. 23(6): p. 850-858 e4]. Related studies have suggested that restriction of αβTCR expression in differentiated iT-cells is driven by TCR allelic exclusion, which prevents erroneous rearrangement of endogenous TCR genes. However, due to the phenomenon of promoter silencing, lentiviral transduction is an inefficient method at promoting stable transgene expression in hiPSC and differentiated cells [Zou, J., et al., Oxidase-deficient neutrophils from X-linked chronic granulomatous disease iPS cells: functional correction by zinc finger nuclease-mediated safe harbor targeting. Blood, 2011. 117(21): p. 5561-72]. In addition, the development of a TiPSC derived from a recombinant heterologous T-cell receptor expressing T-cell would require additional interventions to prevent expression of the native TCR present in the initial T-cell material.

An alternative approach is to genetically engineer the hiPSC to prevent endogenous TCR expression in differentiated iT-cells. This is a challenging approach as it requires the inactivation of multiple genes in both alleles, for example TCR constant domains (TRBC1/2 and TRAC) or possibly genes involved in TCR gene rearrangement (e.g RAG1/2) in conjunction with the heterologous T-cell receptor knock-in. It is however difficult and unpredictable to ascertain what loci to target that will permit TCR expression in differentiated iT-cells. Here we present data based on a minimal editing strategy that targeted insertion of a recombinant heterologous T-cell receptor sequence permits its expression in differentiated iT-cells.

### SUMMARY OF THE INVENTION

According to the present invention there is provided a modified induced pluripotent stem cell iPSC or haemogenic lineage cell comprising at least one heterologous nucleic acid sequence encoding a heterologous T-cell receptor (TCR) integrated at or into a locus in the cell genome.

According to the invention the at least one heterologous nucleic acid sequence encoding a heterologous TCR can be an expressible heterologous nucleic acid sequence. For example the nucleic acid sequence may possess one or more encoding open reading frame and/or one or more regulatory nucleic acid sequences capable of enabling and/or regulating the expression of the encoding gene or genes or one or more encoding open reading within the cell, for example sequences enabling the transcription and/or post transcriptional modification and/or translation of the encoded sequence or gene. Suitable regulatory sequences can comprise any of, transcriptional or translational start, stop or termination sequences, operator sequence, promoter sequence, enhancer sequence, terminal repeats, ribosome binding site, cap sequence, polyA tail sequence.

Preferably the modified induced pluripotent stem cell iPSC or haemogenic lineage cell according to the invention expresses or presents the at least one heterologous TCR encoded by a heterologous TCR coding sequence, preferably expressed or presented at the cell surface. Preferably the at least one heterologous TCR is presented at the cell surface as a membrane bound functional TCR, preferably a membrane anchored heterodimeric TCR protein, preferably an alpha; beta TCR (optionally gamma:delta), preferably comprising variable alpha (a) and beta (p) chains, preferably expressed as a complex with invariant CD3 chains molecules. Preferably the at least one heterologous TCR is capable of binding or specifically binding to a cancer and/or tumour antigen or peptide thereof and/or to tumour and/or cancer cells (e.g. expressing a cancer and/or tumour antigen or peptide thereof) and/or to peptides or antigenic peptides therefrom. Preferably the binding of the TCR promotes activation of the modified induced pluripotent stem cell iPSC or haemogenic lineage cell as herein described and/or activation of T cell activity as herein described.

According to the present invention the modified haemogenic lineage cell may be derived from the modified induced pluripotent stem cell iPSC. For example an unmodified induced pluripotent stem cell iPSC may be modified or engineered, for example recombinantly engineered, to comprise the at least one heterologous nucleic acid sequence encoding a heterologous T-cell receptor (TCR) integrated at or into a locus in the cell genome, for example to produce the modified induced pluripotent stem cell which may then be differentiated into a modified haemogenic lineage cell. Alternatively an unmodified haemogenic lineage cell may be derived from an unmodified induced pluripotent stem cell iPSC, for example by a process of differentiation, and then may be may be modified or engineered to comprise the at least one heterologous nucleic acid sequence encoding a heterologous T-cell receptor (TCR) integrated at or into a locus in the cell genome.

According to the invention the induced pluripotent stem cell iPSC or haemogenic lineage cell, or the modified induced pluripotent stem cell iPSC or haemogenic lineage cell may be selected from any one of;
(a) a mesoderm cell, optionally which may express any one or more of the mesodermal markers, Brachyury, Goosecoid, MixI1, KDR (also known as FLK1 or VEGFR2), FoxA2, GATA6 or PDGF alpha R,
(b) a haemogenic endothelial cell, optionally which may be (a) CD34+ or (b) CD34+CD73− or (c) CD34+CD73−CXCR4−(CD184−),
(c) a haematopoietic progenitor cell, optionally which may be (a) CD34+ or (b) CD34+CD45+ or (c) CD34+ and/or CD45+ in combination with any one or more of CD117+, CD133+, CD45+, FLK+, CD38−, (d) CD34+, CD133+, CD45+, FLK1+, CD38−
(d) a progenitor T cell, optionally which may be (a) CD5+ and/or CD7+, or (b) CD5+ and/or CD7+ in combination with any one or more of: CD44+, CD25+, CD2+, CD45+, CD3−, CD4−, CD8−,
(e) a double positive T cell (DP T cell), optionally which may be (a) CD4+CD8+(b) CD4+CD8+ in combination with any one or more of CD3+, CD28+, CD45+,
(f) a single positive T cell (SP T cell) optionally which may be (a) CD4+(b) CD8+(b) CD4+ or CD8+ in combination with any one or more of CD3+, CD28+, CD45+,
(g) a mature T cell, optionally alpha beta T cell, gamma delta T cell, NKT cell, T helper cell (TH) which are CD4+, cytotoxic T cell (TC) which are CD8+, or
(h) an induced pluripotent stem cell iPSC, optionally a human induced pluripotent stem cell iPSC derived or created from a CD34+ progenitor cell, optionally isolated from umbilical cord blood, further optionally using the pEB-C5 and pEB-TG episomal plasmids.

Accordingly the induced pluripotent stem cell iPSC, is an induced pluripotent stem cell, and/or a human induced pluripotent stem cell iPSC, preferably derived or created from a CD34+ progenitor cell, optionally preferably isolated from umbilical cord blood, further optionally isolated using pEB-C5 and/or pEB-TG episomal plasmids.

In embodiments of the invention, the iPSC may be modified to reduce or eliminate RAG1 expression, for example by inactivation or knock-out of the RAG1 gene.

The modified pluripotent stem cell iPSC or haemogenic lineage cells may be cells of the lymphoid lineage including T cells, Natural Killer T (NKT) cells, and precursors thereof including embryonic stem cells, and pluripotent stem cells (e.g, those from which lymphoid cells may be differentiated). T cells can be lymphocytes that mature in the thymus and are chiefly responsible for cell-mediated immunity and also involved in the adaptive immune system. According to the present invention the T cells can include, but are not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and gamma-delta T cells. Cytotoxic T cells (CTL or killer T cells) are a subset of T-lymphocytes capable of inducing the death of infected somatic or tumour cells. Preferably, the T cells are optionally a CD4+ T cell or a CD8+ T cell or population of such T-cells, optionally CD4+ T cells; or CD8+ T cells, or a mixed population of CD4+ T cells and CD8+ T cells.

According to the present invention the heterologous nucleic acid sequence encoding the heterologous TCR is integrated at or into a locus in the cell genome, i.e. the genome of the induced pluripotent stem cell iPSC or haemogenic lineage cell. The locus may be a specific and/or fixed position on a chromosome for example where a particular gene or genetic marker is located, it may be the specific and/or fixed position of a gene or genetic marker or genetic element and/or a specific fixed position within a gene or genetic element of a gene such as, for example, an intron or exon of the gene or a regulatory element of a gene (e.g. transcriptional or translational start, stop or termination sequences, operator sequence, promoter sequence, enhancer sequence, terminal repeats, ribosome binding site, cap sequence, polyA tail sequence).

According to the invention, the heterologous nucleic acid sequence encoding a heterologous TCR can be integrated at or into one or both alleles of the locus in the cell genome. For example where the locus is a gene or endogenous gene or a specific and/or fixed position on a chromosome, and/or the position where a particular gene or endogenous gene of the induced pluripotent stem cell iPSC or haemogenic lineage cell is located, then the heterologous nucleic acid sequence encoding a heterologous TCR may be integrated at either one or both alleles of the locus.

Accordingly, the locus may be a gene or is within a gene encoding an endogenous protein of the cell or the induced pluripotent stem cell iPSC or haemogenic lineage cell.

According to the invention the heterologous nucleic acid sequence encoding a heterologous TCR may be integrated adjacent to or within a gene encoding an endogenous protein of the cell or the induced pluripotent stem cell iPSC or haemogenic lineage cell. Optionally the heterologous nucleic acid sequence encoding a heterologous TCR may be integrated within an intron or axon of a gene or nuclei acid sequence encoding an endogenous protein of the cell or the induced pluripotent stem cell iPSC or haemogenic lineage cell, optionally a 5′ exon or 3′ exon, preferably 3′ exon.

According to the invention the heterologous nucleic acid sequence encoding a heterologous TCR may be integrated within the 3′ exon before the TAG stop sequence of the gene encoding the endogenous protein. Preferably the integration of the heterologous nucleic acid sequence encoding a heterologous TCR, is non-disruptive to the production of the endogenous protein. For example, the production of the endogenous protein by the modified cell should provide all or substantially all of the endogenous protein sequence, which may be translated and/or produced and/or processed equivalently to the native protein. For example the gene encoding the endogenous protein may still be capable of transcription and/or post transcriptional processing and/or translation produce the endogenous protein sequence or substantially all of the endogenous protein sequence which may optionally be post translationally processed and/or trafficked in the cell. Preferably the integration of the heterologous nucleic acid sequence encoding a heterologous TCR, does not result in the deletion or substitution of the gene encoding the endogenous protein, and/or the deletion and/or inactivation of the produced endogenous protein, it may result in the concatenation of the heterologous nucleic acid sequence encoding a heterologous TCR with the nucleic acid sequence or gene encoding the endogenous protein, optionally such that a fusion gene results and/or the gene products are produced as a fusion protein.

According to the invention the modified induced pluripotent stem cell iPSC or haemogenic lineage cell may comprise a fusion sequence between the nucleic acid encoding the heterologous TCR and the nucleic acid encoding the endogenous protein, for example as generated by the integration of the nucleic acid encoding the heterologous TCR, for example a fusion gene or sequence or multicistronic fusion gene or sequence between the nucleic acid encoding the heterologous TCR and the nucleic acid encoding the endogenous protein, for example wherein the expression of the heterologous TCR is linked to the expression of the endogenous protein and/or wherein the heterologous TCR may co-transcribed and/or co-translated and/or co-expressed with the gene product of the gene at the locus, e.g. the nucleic acid encoding the endogenous gene.

Accordingly the invention provides a modified induced pluripotent stem cell iPSC or haemogenic lineage cell in which the nucleic acid encoding the heterologous TCR may be connected to a nucleic acid encoding an endogenous protein by a nucleic acid sequence encoding a peptide comprising an enzymatic cleavage site and/or a nucleic acid sequence which mediates ribosome-skipping.

Accordingly the invention provides a modified induced pluripotent stem cell iPSC or haemogenic lineage cell in which the nucleic acid encoding the heterologous TCR may be connected to a nucleic acid encoding an endogenous protein by at least one, optionally two, nucleic acid sequence encoding a peptide comprising an enzymatic cleavage site and/or at least one, optionally two, nucleic acid sequence which mediates ribosome-skipping. The nucleic acid sequence which mediates ribosome-skipping may be either a T2A and/or P2A skip sequence. The nucleic acid sequence encoding a peptide comprising an enzymatic cleavage site may encode a furin cleavage site, the sequence of the encoded furin site may be preferably RAKR.

According to the invention, the nucleic acid encoding the heterologous TCR may comprise a coding sequence of a TCRα and/or TCRβ chain, optionally with at least one, optionally two intervening nucleic acid sequence encoding a peptide comprising an enzymatic cleavage site and/or at least one, optionally two, nucleic acid sequence which mediates ribosome-skipping. The nucleic acid sequence which mediates ribosome-skipping may be either a T2A and/or P2A skip sequence. The nucleic acid sequence encoding a peptide comprising an enzymatic cleavage site may encode a furin cleavage site, the sequence of the encoded furin site may be preferably RAKR.

Accordingly, the transcription and/or expression of the heterologous TCR and the endogenous protein of the modified induced pluripotent stem cell iPSC or haemogenic lineage cell according to the invention may be from the same promoter. Accordingly the heterologous nucleic acid sequence encoding a heterologous TCR and nucleic acid sequence or gene encoding the endogenous protein may share the same regulatory sequences for transcription and/or post transcriptional modification or co-transcriptional modification and/or translation, regulatory sequences as referred to herein. Post transcriptional modification or co-transcriptional may include processing of an RNA primary transcript following transcription to produce a mature, functional RNA molecule and/or may include any of mRNA processing and/or 5′ processing and/or Capping and/or 3′ processing and/or cleavage and polyadenylation and/or introns splicing and/or histone mRNA processing

The present invention provides a modified induced pluripotent stem cell iPSC or haemogenic lineage cell according to the invention, wherein the heterologous TCR is expressed as a fusion protein with an endogenous protein, optionally connected by a peptide comprising an enzymatic cleavage site, preferably a furin cleavage site, preferably RAKR and/or ribosome skip sequence, preferably T2A and/or P2A skip sequence.

Accordingly, the fusion protein may be processed in the cell to release the nascent, free or native heterologous TCR, optionally by cleavage of the enzyme cleavage site or furin cleavage site and/or removal the skip sequence peptides optionally performed in the endoplasmic reticulum of the cell.

The present invention provides a modified induced pluripotent stem cell iPSC or haemogenic lineage cell according to the invention wherein the heterologous TCR is expressed and/or presented in the cell as a nascent heterologous TCR comprising a TCR alpha chain and TCR beta chain, preferably expressed and/or presented at the cell surface. The nascent protein may be separated from the fusion protein and thereby from the endogenous protein for example during post translational processing in the cell, such is in the cell endoplasmic reticulum or golgi, it is then optionally trafficked and/or presented at the cell surface as a membrane bound functional TCR, preferably a membrane anchored heterodimeric TCR protein, preferably an alpha; beta TCR optionally capable of binding or specifically binding to a cancer or tumour antigen or peptide thereof and/or to tumour and/or cancer cells and/or tumour and/or cancer tissue and/or to peptides or antigenic peptides therefrom and/or promoting activation of the modified induced pluripotent stem cell iPSC or haemogenic lineage cell as herein described and/or activating T cell activity as herein described.

According to the present invention the endogenous protein can be a protein that is trafficked to the cell surface, optionally via the secretory pathway and/or to the plasma membrane of the cell. Preferably the endogenous protein is a membrane protein and/or transmembrane protein, optionally a receptor protein, preferably a receptor protein tyrosine phosphatase (PTP), preferably CD45 or protein tyrosine phosphatase receptor type C or PTPRC.

According to the invention the least one heterologous nucleic acid sequence encoding a heterologous T-cell receptor (TCR) can be integrated at or into a locus in the cell genome where the locus is or is in a gene encoding a membrane protein or transmembrane protein, optionally a receptor protein, preferably a receptor protein tyrosine phosphatase (PTP), preferably CD45 or protein tyrosine phosphatase receptor type C or PTPRC. Preferably the locus is or is in the PTPRC (CD45) gene on chromosome 1.

According to the invention the least one heterologous nucleic acid sequence encoding a heterologous T-cell receptor (TCR) can be integrated at or into a locus in the cell genome where the locus or site of integration is at exon 33 of the PTPRC (CD45) gene, preferably in the PTPRC (CD45) gene on chromosome 1, optionally before the TAG stop codon or immediately adjacent to and/or before the TAG stop codon.

The present invention provides a modified induced pluripotent stem cell iPSC or haemogenic lineage cell according to the present invention, wherein the fusion gene or sequence or multicistronic fusion gene or sequence comprises the nucleic acid encoding the heterologous TCR and nucleic acid encoding PTPRC with an intervening nucleic acid sequence encoding a peptide comprising an enzymatic cleavage site, preferably a furin cleavage site, and/or nucleic acid sequence which mediates ribosome-skipping, preferably selected from a T2A and/or P2A skip sequence and wherein the nucleic acid sequence encoding the heterologous TCR comprises the coding sequence of a TCRα and TCRβ chain, preferably with an intervening nucleic acid sequence encoding a peptide comprising an enzymatic cleavage site, preferably a furin cleavage site, and/or nucleic acid sequence which mediates ribosome-skipping, preferably selected from T2A and/or P2A skip sequence.

### TCR Binding

According to the invention the heterologous TCR may bind or specifically bind to an antigen or peptide antigen which is any of:
(a) a cancer and/or tumour antigen or peptide antigen thereof, or
(b) a cancer and/or tumour antigen or peptide antigen thereof associated with a cancerous condition and/or presented by tumour or cancer cell or tissue.

According to the present invention the heterologous TCR may bind or specifically and/or selectively bind to an antigen or peptide antigen thereof, for example to a cancer and/or tumour antigen or peptide antigen thereof, optionally presented by a cancer and/or tumour and/or in complex with a peptide presenting molecule, for example major histocompatibility complex (MHC) or an HLA, alternatively presented without a peptide presenting molecule (for example as an endogenously expressed cancer and/or tumour cell surface antigen or peptide antigen). The cancer and/or tumour antigen or peptide antigen thereof can be an antigen or peptide antigen thereof expressed by cancer and/or tumour cells or tissue at a higher level than on normal cells or tissue, preferably a significantly higher level, for example 100, 1000, 10,000, 100,000, 1000,000 or greater times higher, preferably the antigen or peptide antigen thereof is not expressed by normal cells or tissue. The cancer and/or tumour antigen or peptide antigen thereof may be any of a cancer-testis antigen, NY-ESO-1, MART-1 (melanoma antigen recognized by T cells), WT1 (Wilms tumor 1), gp100 (glycoprotein 100), tyrosinase, PRAME (preferentially expressed antigen in melanoma), p53, HPV-E6/HPV-E7 (human papillomavirus), HBV, TRAIL, DR4, Thyroglobin, TGFBII frameshift antigen, LAGE-1A, KRAS, CMV (cytomegalovirus), CEA (carcinoembryonic antigen), AFP (α-fetoprotein), a MAGE, melanoma associated antigen or member of the MAGEA gene family, MAGE-A1, MAGE-A2, MAGE-A3, MAGE-A4, MAGE-A6, MAGE-A8, and MAGE-A9, MAGE-A10, or MAGE-A12, or peptide antigen thereof.

According to the invention the TCR may bind MAGE A4, or an antigenic peptide thereof, for example Human MAGE A4 or MAGE A4 of SEQ ID NO: 1 or an antigenic peptide thereof. The TCR may bind to an antigenic peptide of MAGE A4 comprising SEQ ID NO: 2, GVYDGREHTV. Alternatively the TCR may bind alpha fetoprotein (AFP), or an antigenic peptide thereof, for example alpha fetoprotein (AFP) of SEQ ID NO: 20 or a peptide antigen of AFP or a peptide antigen of AFP comprising FMNKFIYEI (SEQ ID No: 21) or residues 158-166 derived from alpha fetoprotein (AFP) SEQ ID NO: 20.

The TCR may bind to an AFP antigenic peptide comprising;
(a) residues 158-166 of SEQ ID NO:20 (AFP158),
(b) residues 137-145 of SEQ ID NO:20 (AFP137),
(c) residues 325-334 of SEQ ID NO:20 (AFP325),
(d) residues 542-550 of SEQ ID NO:20 (AFP542), or
(e) residues FMNKFIYEI (SEQ ID No: 21).

### Specific Binding TCR

According to the invention the heterologous TCR may bind or specifically bind an antigen or peptide antigen thereof for example a cancer and/or tumour antigen or peptide antigen thereof as described herein, optionally associated with a cancerous condition and/or presented by tumour or cancer cell or tissue.

Specificity describes the strength of binding between the heterologous TCR and a specific target cancer and/or tumour antigen or peptide antigen thereof and may be described by a dissociation constant, Kd, the ratio between bound and unbound states for the receptor-ligand system. Additionally, the fewer different cancer and/or tumour antigens or peptide antigen thereof the heterologous TCR can bind, the greater its binding specificity.

According to the invention the heterologous TCR may bind to less than 10, 9, 8, 7, 6, 5, 4, 3, 2 different cancer and/or tumour antigens or peptide antigen thereof.

According to the invention the heterologous TCR may bind, an antigen or peptide antigen thereof, for example to a cancer and/or tumour antigen or peptide antigen, for example MAGE A4 of SEQ ID NO: 1, an antigenic peptide of MAGE A4 comprising SEQ ID NO: 2, alpha fetoprotein (AFP) of SEQ ID NO: 20 or a peptide antigen of AFP or a peptide antigen of AFP comprising FMNKFIYEI (SEQ ID No: 21), with a dissociation constant of between, 0.01 μM and 100 μM, between 0.01 μM and 50 μM, between 0.01 μM and 20 μM, between 0.05 μM and 20 μM or of 0.01, 0.02, 0.03, 0.04, 0.05, 0.06, 0.07, 0.08, 0.09, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, 10.0 μM; or between 10 μM and 1000 μM, between 10 μM and 500 μM, between 50 μM and 500 μM or of 10, 20 30, 40, 50 60, 70, 80, 90, 100 μM, 150 μM, 200 μM, 250 μM, 300 μM, 350 μM, 400 μM, 450 μM, 500 μM; optionally measured with surface plasmon resonance, optionally at 25° C., optionally between a pH of 6.5 and 6.9 or 7.0 and 7.5. The dissociation constant, K_{D} or k_{off}/kₒₙ may be determined by experimentally measuring the dissociation rate constant, k_{off}, and the association rate constant, kₒₙ. A TCR dissociation constant may be measured using a soluble form of the TCR, wherein the TCR comprises a TCR alpha chain variable domain and a TCR beta chain variable domain.

According to the invention the heterologous T cell receptor (TCR), and/or modified cells comprising the heterologous T cell receptor (TCR) may specifically and/or with high affinity, and/or selectively bind, an antigen or peptide antigen thereof, for example to a cancer and/or tumour antigen or peptide antigen, as herein described in complex with or presented by a peptide presenting molecule for example major histocompatibility complex (MHC) or an HLA, optionally class I or II, for example with HLA-A2, or selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642; for example with a dissociation constant as herein above described, preferably of between 0.01 μM and 100 μM such as 50 μM, 100 μM, 200 μM, 500 μM, preferably between 0.05 μM to 20.0 μM.

According to the invention the heterologous T cell receptor (TCR), and/or modified cells comprising the heterologous T cell receptor (TCR) may specifically and/or with high affinity, and/or selectively bind, MAGE A4 (SEQ ID NO: 1) a peptide antigen thereof or an antigenic peptide thereof comprising SEQ ID NO: 2, GVYDGREHTV optionally in complex with HLA-A*02, optionally selected from HLA*02, HLA-A*02:01, HLA-A*02:02, HLA-A*02:03, HLA-A*02:04, HLA-A*02:06, HLA-A*02:642 or HLA-A*02:07, preferably HLA-A*02:01 or HLA-A*02; for example with a dissociation constant as herein above described, preferably of between 0.01 μM and 100 μM such as 50 μM, 100 μM, 200 μM, 500 μM, preferably between 0.05 μM to 20.0 μM.

According to the invention the heterologous T cell receptor (TCR), and/or modified cells comprising the heterologous T cell receptor (TCR) may specifically and/or with high affinity, and/or selectively bind, to AFP (SEQ ID NO: 20) a peptide antigen thereof or peptide antigen comprising the sequence FMNKFIYEI (SEQ ID No: 21), optionally in complex with HLA-A2, or with any HLA selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642; for example with a dissociation constant as herein above described, preferably of between 0.01 μM and 100 μM such as 50 μM, 100 μM, 200 μM, 500 μM, preferably between 0.05 μM to 20.0 μM.

Alternatively the heterologous T cell receptor (TCR), and modified cells comprising the heterologous T cell receptor (TCR) may bind or specifically and/or selectively bind and/or bind with high affinity to an antigen or peptide antigen thereof, for example to a cancer and/or tumour antigen or peptide antigen, as herein described as an endogenously expressed cancer and/or tumour cell surface antigen or peptide antigen (e.g. AFP or MAGE A4 or antigenic peptides thereof) optionally wherein the binding is independent of presentation of the cell surface antigen as a complex with an peptide-presenting or antigen-presenting molecule, for example major histocompatibility complex (MHC) or human leukocyte antigen (HLA) or major histocompatibility complex class related protein (MR)1; for example with a dissociation constant as herein above described, preferably of between 0.01 μM and 100 μM such as 50 μM, 100 μM, 200 μM, 500 μM, preferably between 0.05 μM to 20.0 μM.

According to the present invention the TCR binding may be specific for one antigen or peptide antigen thereof, for example a cancer and/or tumour antigen or peptide antigen thereof, as herein described (e.g. AFP or MAGE A4 or antigenic peptides thereof), optionally as expressed on a cancer and/or tumour cell surface, in comparison to a closely related cancer and/or tumour antigen or peptide antigen sequence. The closely related cancer and/or tumour antigen or peptide antigen sequence may be of similar or identical length and/or may have a similar number or identical number of amino acid residues. The closely related peptide antigen sequence may share between 50 or 60 or 70 or 80 to 90% identity, preferably between 80 to 90% identity and/or may differ by 1, 2, 3 or 4 amino acid residues. For example, the closely related peptide sequence may be derived from the polypeptide sequence comprising the sequence or having the sequence GVYDGREHTV, SEQ ID NO: 2 or FMNKFIYEI, SEQ ID No: 21.

The binding affinity may be determined by equilibrium methods (e.g. enzyme-linked immunosorbent assay (ELISA) or radioimmunoassay (RIA)), or kinetics (e.g. BIACORE™ analysis). Avidity is the sum total of the strength of binding of two molecules to one another at multiple sites, e.g. taking into account the valency of the interaction. According to the invention the modified pluripotent stem cell iPSC or haemogenic lineage cells may demonstrate improved affinity and/or avidity to a cancer and/or tumour antigen or peptide antigen thereof, or a cancer and/or tumour antigen or peptide antigen thereof presented by tumour of cancer cell or tissue and recognised by the heterologous TCR in comparison in comparison to cells lacking the heterologous TCR or having an alternative heterologous TCR or CAR.

Selective binding denotes that the heterologous TCR binds with greater affinity to one cancer and/or tumour antigen or peptide antigen thereof in comparison to another. Selective binding is denoted by the equilibrium constant for the displacement by one ligand antigen of another ligand antigen in a complex with the heterologous TCR.

### Heterologous TCR

According to the present invention the modified pluripotent stem cell iPSC or haemogenic lineage cells comprising at least one heterologous nucleic acid sequence encoding a heterologous TCR integrated into a locus in the cell genome can express a heterologous T cell receptor (TCR). Upon binding to the antigen, the modified pluripotent stem cell iPSC or haemogenic lineage cells can exhibit T cell effector functions and/or cytolytic effects towards cells bearing the antigen and/or undergo proliferation and/or cell division. In certain embodiments, the modified pluripotent stem cell iPSC or haemogenic lineage cells comprising the TCR exhibits comparable or better therapeutic potency compared to T cells comprising either a transduced TCR or a chimeric antigen receptor (CAR) targeting the same cancer and/or tumour antigen and/or peptide (antigenic peptide). Activated modified pluripotent stem cell iPSC or haemogenic lineage cells can secrete anti-tumour cytokines which can include, but are not limited to, TNFalpha, IFNγ and IL2.

The term “heterologous” or “exogenous” refers to a polypeptide or nucleic acid that is foreign to a particular biological system, such as a cell or host cell, and is not naturally present in that system and which may be introduced to the system by artificial or recombinant means. Accordingly, the expression of a TCR which is heterologous, may thereby alter the immunogenic specificity of the pluripotent stem cell iPSC or haemogenic lineage cells, for example T cells, so that they recognise or display improved recognition for one or more tumour or cancer antigens and/or peptides as herein described that are present on the surface of the cancer cells of an individual with cancer. The modification of immunogenic cells or T cells and their subsequent expansion may be performed in vitro and/or ex vivo.

### TCR Structure

Preferably, the heterologous TCR is not naturally or endogenously expressed by the induced pluripotent stem cell iPSC or haemogenic lineage cells, i.e. prior to modification, (i.e. the TCR is exogenous or heterologous). A heterologous TCR may include αβTCR heterodimers. A heterologous TCR may be a recombinant or synthetic or artificial TCR i.e. a or TCR that does not exist in nature. For example, a heterologous TCR may be engineered to increase its affinity or avidity for a specific cancer and/or tumour antigen or peptide antigen thereof (i.e. an affinity enhanced TCR or specific peptide enhanced affinity receptor (SPEAR) TCR). The affinity enhanced TCR or (SPEAR) TCR may comprise one or more mutations relative to a naturally occurring TCR, for example, one or more mutations in the hypervariable complementarity determining regions (CDRs) of the variable regions of the TCR α and β chains. These mutations may increase the affinity of the TCR for a cancer and/or tumour antigen or peptide antigen thereof, as herein described or MHCs that display a cancer and/or tumour antigen or peptide antigen thereof, as herein described, optionally when expressed by tumour and/or cancer cells. Suitable methods of generating affinity enhanced or matured TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to tumour or cancer cells expressing the tumour antigen of the MAGE family, or AFP or peptide antigens thereof as described herein.

### MAGEA4/AFP TCR

According to the invention the heterologous TCR may be a MAGE A4 TCR, which may comprise the α chain reference amino acid sequence of SEQ ID NO: 5 or a variant thereof and the β chain reference amino acid sequence of SEQ NO: 7 or a variant thereof. Alternatively the heterologous TCR may be an AFP TCR which may comprise the α chain reference amino acid sequence of SEQ ID NO: 22 or a variant thereof and the β chain reference amino acid sequence of SEQ NO: 23 or a variant thereof.

A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence (for example, with respect to either a chain reference sequence and/or β chain reference sequence). The TCR may be encoded by the α chain reference nucleotide sequence of SEQ ID NO: 6 or a variant thereof and the β chain reference nucleotide sequence of SEQ NO: 8 or a variant thereof (MAGE A4) or by the α chain reference nucleotide sequence of SEQ ID NO: 24 or a variant thereof and the β chain reference nucleotide sequence of SEQ NO: 25 (AFP) or a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence (for example, with respect to either a chain reference sequence and/or β chain reference sequence for an AFP or MAGE A4 TCR).

### TCR CDRs

According to the present invention, for example for MAGE A4, the heterologous TCR may comprise a TCR alpha chain variable domain and a TCR beta chain variable domain, wherein:
(i) the alpha chain variable domain comprises CDRs having the sequences
VSPFSN (αCDR1), SEQ ID NO:11 or amino acids 48-53 of SEQ ID NO:5,
LTFSEN (αCDR2), SEQ ID NO:12 or amino acids 71-76 of SEQ ID NO:5, and
CVVSGGTDSWGKLQF (αCDR3), SEQ ID NO:13 or amino acids 111-125 of SEQ ID NO:5, and/or
(ii) the beta chain variable domain comprises CDRs having the sequences
KGHDR (βCDR1), SEQ ID NO:14 or amino acids 46-50 of SEQ ID NO:7,
SFDVKD (βCDR2), SEQ ID NO:15 or amino acids 68-73 of SEQ ID NO:7, and
CATSGQGAYEEQFF (βCDR3), SEQ ID NO:16 or amino acids 110-123 of SEQ ID NO:7 or sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto, optionally 100% sequence identity thereto, respectively.

According to the invention, for example for AFP, the heterologous TCR can comprise a TCR alpha chain variable domain and a TCR beta chain variable domain, wherein:
(i) the alpha chain variable domain comprises CDRs having the sequences
DRGSQS (αCDR1), SEQ ID NO:26 or amino acids 27-32 of SEQ ID NO:22, or sequence having at least 50% sequence identity thereto,
IYSNGD (αCDR2), SEQ ID NO:27 or amino acids 50-55 of SEQ ID NO:22, or sequence having at least 50% sequence identity thereto, and
AVNSDSGYALNF (αCDR3), SEQ ID NO:28 or amino acids 90-101 of SEQ ID NO:22, or sequence having at least 50% sequence identity thereto, and
(ii) the beta chain variable domain comprises CDRs having the sequences
SGDLS (βCDR1), SEQ ID NO:29 or amino acids 27-31 of SEQ ID NO:23, or sequence having at least 50% sequence identity thereto,
YYNGEE (βCDR2), SEQ ID NO:30 or amino acids 49-54 of SEQ ID NO:23, or sequence having at least 50% sequence identity thereto, and
ASSLGGESEQY (βCDR3), SEQ ID NO:31 or amino acids 92-102 of SEQ ID NO:23; or sequences having at least 50% sequence identity thereto; optionally the sequence identity may be any of least 50%, at least 55%, at least 60%, at least 65%, at least 70%, at least 75%, at least 80%, at least 85%, at least 90%, at least 95%, at least 98% or at least 99% sequence identity, optionally 100% sequence identity thereto respectively.

### TCR Variable Domains

According to the invention, the heterologous TCR may comprise a TCR, e.g. MAGE A4 TCR, in which the alpha chain variable domain comprises an amino acid sequence that has at least 50%, 55%, 60%, 65%, 70%, 75%, 80%, 85%, 90%, 95%, 96%, 97%, 98% or 99% or 100% identity to SEQ ID NO:9 or the sequence of amino acid residues 1-136 of SEQ ID NO:5, and/or the beta chain variable domain comprising an amino acid sequence that has at least 50%, 55%, 60%, 65%, 70%, 75%, 80%, 85%, 90%, 95%, 96%, 97%, 98% or 99% or 100% identity to SEQ ID NO:10 or the sequence of amino acid residues 1-133 of SEQ ID NO:7.

According to the invention, the heterologous TCR, e.g. AFP TCR, may comprise a TCR in which the alpha chain variable domain comprises an amino acid sequence that has at least 50%, 55%, 60%, 65%, 70%, 75%, 80%, 85%, 90%, 95%, 96%, 97%, 98% or 99% or 100% identity to the sequence of amino acid residues 1-112 of SEQ ID NO: 22, and/or the beta chain variable domain comprising an amino acid sequence that has at least 50%, 55%, 60%, 65%, 70%, 75%, 80%, 85%, 90%, 95%, 96%, 97%, 98% or 99% or 100% identity to the sequence of amino acid residues 1-112 of SEQ ID NO: 23.

### MAGE A4 & AFP TCR CDRs and Frameworks

The terms “progenitor TCR” or “parental TCR”, is used herein to refer to a TCR comprising the MAGE A4 TCR α chain and MAGE A4 TCR β chain of SEQ ID NOs: 5 and 7 respectively, or a TCR comprising the AFP TCR α chain and AFP TCR β chain of SEQ ID NOs: 22 and 23 respectively.

It is desirable to provide heterologous TCRs that are mutated or modified relative to the progenitor TCR that have an equal, equivalent or higher affinity and/or an equal, equivalent or slower off-rate for the peptide-HLA complex than the progenitor TCR. According to the invention the heterologous TCR may have more than one mutation present in the alpha chain variable domain and/or the beta chain variable domain relative to the progenitor TCR and may be denoted, “engineered TCR” or “mutant TCR”. These mutation(s) may improve the binding affinity and/or specificity and/or selectivity and/or avidity for target AFP or MAGE A4 or peptide antigens thereof. In certain embodiments, there are 1, 2, 3, 4, 5, 6, 7 or 8 mutations in alpha chain variable domain, for example 4 or 8 mutations, and/or 1, 2, 3, 4 or 5 mutations in the beta chain variable domain, for example 5 mutations. In some embodiments, the α chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 9 (MAGE A4) or amino acids 1-112 of of SEQ ID NO: 22 (AFP). In some embodiments, the β chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 10 (MAGE A4) or amino acids 1-112 of SEQ ID NO: 23 (AFP).

According to the invention the heterologous TCR, (e.g. MAGE A4 TCR), may comprise a TCR in which, the alpha chain variable domain comprises SEQ ID NO: 9 or the amino acid sequence of amino acid residues 1-136 of SEQ ID NO:5 or an amino acid sequence in which amino acid residues 1-47, 54-70, 77-110 and 126-136 thereof have at least 70%, 75%, 80%, 85%, 90%, 95%, 96%, 97%, 98% or 99% identity to the sequence of amino acid residues 1-47, 54-70, 77-110 and 126-136 respectively of SEQ ID NO:9 and/or in which amino acid residues 48-53, 71-76 and 111-125, CDR 1, CDR 2, CDR 3 respectively, have at least 70%, 75%, 80%, 85%, 90%, 95%, 96%, 97%, 98% or 99% identity to the sequence of amino acid residues 48-53, 71-76 and 111-125, CDR 1, CDR 2, CDR 3, respectively of SEQ ID NO:9.

According to the invention, the heterologous TCR may comprise a TCR in which, in the alpha chain variable domain, the sequence of:
(i) amino acid residues 1-47 thereof may have (a) at least 70%, 75%, 80%, 85%, 90% or 95% identity to the sequence of amino acid residues 1-47 of SEQ ID NO:9 or (b) may have one, two or three amino acid residues inserted or deleted relative to residues 1-47 of SEQ ID NO:9,
(ii) amino acid residues 48-53 is VSPFSN, CDR 1, SEQ ID NO:11 or amino acids 48-53 of SEQ ID NO:9,
(iii) amino acid residues 54-70 thereof may have (a) at least 70%, 75%, 80%, 85%, 90% or 95% identity to the sequence of amino acid residues 54-70 of SEQ ID NO: 9 or (b) may have one, two or three amino acid residues inserted or deleted relative to the sequence of amino acid residues 54-70 of SEQ ID NO: 9,
(iv) amino acid residues 71-76 may be LTFSEN, CDR 2, SEQ ID NO:12 or amino acids 71-76 of SEQ ID NO:9,
(v) amino acid residues 77-110 thereof may have at least 70%, 75%, 80%, 85%, 90% or 95% identity to the sequence of amino acid residues 77-110 of SEQ ID NO:9 or may have one, two or three insertions, deletions or substitutions relative to the sequence of amino acid residues 77-110 of SEQ ID NO:9,
(vi) amino acids 111-125 may be CVVSGGTDSWGKLQF, CDR 3, SEQ ID NO:13 or amino acids 111-125 of SEQ ID NO:9,
(vii) amino acid residues 126-136 thereof may have at least 70%, 75%, 80%, 85%, 90% or 95% identity to the sequence of amino acid residues 126-136 of SEQ ID NO: 9 or may have one, two or three insertions, deletions or substitutions relative to the sequence of amino acid residues 126-136 of SEQ ID NO:9.

According to the invention, the heterologous TCR, (e.g. MAGE A4 TCR), may comprise a TCR in which, in the beta chain variable domain comprises the amino acid sequence of SEQ ID NO:10, or an amino acid sequence in which amino acid residues 1-45, 51-67, 74-109, 124-133 thereof have at least 70%, 75%, 80%, 85%, 90% or 95% identity to the sequence of amino acid residues 1-45, 51-67, 74-109, 124-133 respectively of SEQ ID NO:10 and in which amino acid residues 46-50, 68-73 and 110-123 have at least 70%, 75%, 80%, 85%, 90% or 95% identity to the sequence of amino acid residues 46-50, 68-73 and 110-123, CDR 1, CDR 2, CDR 3, respectively of SEQ ID NO:10.

According to the invention, the heterologous TCR may comprise a TCR in which, in the beta chain variable domain, the sequence of:
(i) amino acid residues 1-45 thereof may have (a) at least 70%, 75%, 80%, 85%, 90% or 95% identity to the sequence of amino acid residues 1-45 of SEQ ID NO:10 or (b) may have one, two or three amino acid residues inserted or deleted relative to residues 1-45 of SEQ ID NO:10,
(ii) amino acid residues 46-50 is KGHDR, CDR 1, SEQ ID NO:14 or amino acids 46-50 of SEQ ID NO:10,
(iii) amino acid residues 51-67 thereof may have (a) at least 70%, 75%, 80%, 85%, 90% or 95% identity to the sequence of amino acid residues 51-67 of SEQ ID NO:10 or (b) may have one, two or three amino acid residues inserted or deleted relative to the sequence of amino acid residues 51-67 of SEQ ID NO:10,
(iv) amino acid residues 68-73 may be SFDVKD, CDR 2, SEQ ID NO:15 or amino acids 68-73 of SEQ ID NO:10,
(v) amino acid residues 74-109 thereof may have at least 70%, 75%, 80%, 85%, 90% or 95% identity to the sequence of amino acid residues 74-109 of SEQ ID NO:10 or may have one, two or three insertions, deletions or substitutions relative to the sequence of amino acid residues 74-109 of SEQ ID NO:10;
(vi) amino acids 110-123 may be CATSGQGAYEEQFF, CDR 3, SEQ ID NO:16 or amino acids 110-123 of SEQ ID NO:10,
(vii) amino acid residues 124-133 thereof may have at least 70%, 75%, 80%, 85%, 90% or 95% identity to the sequence of amino acid residues 124-133 of SEQ ID NO:10 or may have one, two or three insertions, deletions or substitutions relative to the sequence of amino acid residues 124-133 of SEQ ID NO:10.

According to the invention, the heterologous TCR may comprise a TCR which comprises an alpha chain variable domain of SEQ ID NO: 9 and/or a beta chain variable domain of SEQ ID NO: 10. According to the invention, the TCR may comprise a TCR which comprises an alpha chain of SEQ ID NO: 5 and/or a beta chain of SEQ ID NO: 7.

### AFP TCR CDRs and Frameworks

According to the invention the heterologous TCR, (e.g. AFP TCR), may comprise a TCR in which, the alpha chain variable domain comprises the amino acid sequence of amino acid residues 1-112 of SEQ ID NO:22, or an amino acid sequence in which amino acid residues 1-26, 33-49, 56-89 and 102-112 thereof have at least 70%, 75%, 80%, 85%, 90%, 95%, 96%, 97%, 98% or 99% identity to the sequence of amino acid residues 1-26, 33-49, 56-89 and 102-112 respectively of SEQ ID NO:22 and/or in which amino acid residues 27-32, 50-55, 90-101, CDR 1, CDR 2, CDR 3 respectively, have at least 70%, 75%, 80%, 85%, 90%, 95%, 96%, 97%, 98% or 99% identity to the sequence of amino acid residues 27-32, 50-55, 90-101, CDR 1, CDR 2, CDR 3, respectively of SEQ ID NO:22.

According to the invention, the heterologous TCR, (e.g. AFP TCR), may comprise a TCR in which, in the alpha chain variable domain, the sequence of:
(i) amino acid residues 1-26 thereof may have (a) at least 70%, 75%, 80%, 85%, 90% or 95% identity to the sequence of amino acid residues 1-26 of SEQ ID NO:22 or (b) may have one, two or three amino acid residues inserted or deleted relative to residues 1-26 of SEQ ID NO:22,
(ii) amino acid residues 27-32 is DRGSQS, αCDR 1, SEQ ID NO:26 or amino acids 27-32 of SEQ ID NO:22,
(iii) amino acid residues 33-49 thereof may have (a) at least 70%, 75%, 80%, 85%, 90% or 95% identity to the sequence of amino acid residues 33-49 of SEQ ID NO: 22 or (b) may have one, two or three amino acid residues inserted or deleted relative to the sequence of amino acid residues 33-49 of SEQ ID NO: 22,
(iv) amino acid residues 50-55 may be IYSNGD, αCDR 2, SEQ ID NO:27 or amino acids 50-55 of SEQ ID NO:22,
(v) amino acid residues 56-89 thereof may have at least 70%, 75%, 80%, 85%, 90% or 95% identity to the sequence of amino acid residues 56-89 of SEQ ID NO:22 or may have one, two or three insertions, deletions or substitutions relative to the sequence of amino acid residues 56-89 of SEQ ID NO:22,
(vi) amino acids 90-101 may be AVNSDSGYALNF, αCDR 3, SEQ ID NO:28 or amino acids 90-101 of SEQ ID NO:22,
(vii) amino acid residues 102-112 thereof may have at least 70%, 75%, 80%, 85%, 90% or 95% identity to the sequence of amino acid residues 102-112 of SEQ ID NO: 22 or may have one, two or three insertions, deletions or substitutions relative to the sequence of amino acid residues 102-112 of SEQ ID NO: 22.

According to the invention, the TCR, (e.g. AFP TCR), may comprise a TCR in which, in the beta chain variable domain comprises the amino acid sequence of amino acid residues 1-112 of SEQ ID NO: 23, or an amino acid sequence in which amino acid residues 1-26, 32-48, 55-91, 103-112 thereof have at least 70%, 75%, 80%, 85%, 90% or 95% identity to the sequence of amino acid residues 1-26, 32-48, 55-91, 103-112 respectively of SEQ ID NO: 23 and in which amino acid residues 27-31, 49-54 and 92-102 have at least 70%, 75%, 80%, 85%, 90% or 95% identity to the sequence of amino acid residues 27-31, 49-54 and 92-102, βCDR 1, βCDR 2, βCDR 3, respectively of SEQ ID NO: 23.

According to the invention, the TCR may comprise a TCR in which, in the beta chain variable domain, the sequence of:
(i) amino acid residues 1-26 thereof may have (a) at least 70%, 75%, 80%, 85%, 90% or 95% identity to the sequence of amino acid residues 1-26 of SEQ ID NO: 23 or (b) may have one, two or three amino acid residues inserted or deleted relative to residues 1-26 of SEQ ID NO: 23,
(ii) amino acid residues 27-31 is SGDLS, βCDR 1, SEQ ID NO:29 or amino acids 27-31 of SEQ ID NO: 23,
(iii) amino acid residues 32-48 thereof may have (a) at least 70%, 75%, 80%, 85%, 90% or 95% identity to the sequence of amino acid residues 32-48 of SEQ ID NO: 23 or (b) may have one, two or three amino acid residues inserted or deleted relative to the sequence of amino acid residues 32-48 of SEQ ID NO: 23,
(iv) amino acid residues 49-54 may be YYNGEE, CDR 2, SEQ ID NO: 30 or amino acids 49-54 of SEQ ID NO: 23,
(v) amino acid residues 55-91 thereof may have at least 70%, 75%, 80%, 85%, 90% or 95% identity to the sequence of amino acid residues 55-91 of SEQ ID NO: 23 or may have one, two or three insertions, deletions or substitutions relative to the sequence of amino acid residues 55-91 of SEQ ID NO: 23
(vi) amino acids 92-102 may be ASSLGGESEQY, βCDR 3, SEQ ID NO: 31 or amino acids 92-102 of SEQ ID NO: 23,
(vii) amino acid residues 103-112 thereof may have at least 70%, 75%, 80%, 85%, 90% or 95% identity to the sequence of amino acid residues 103-112 of SEQ ID NO: 23 or may have one, two or three insertions, deletions or substitutions relative to the sequence of amino acid residues 103-112 of SEQ ID NO: 23.

According to the invention the heterologous TCR may comprise a TCR in which the alpha chain comprises amino acid residues of SEQ ID No: 41, and the beta chain variable domain comprises amino acid residues of SEQ ID No: 23 or SEQ ID NO: 42.

### MUTANT AFP TCRs

Embodiments of the invention can include heterologous TCRs which are mutated relative to the parental AFP TCR, (in which the alpha chain comprises amino acid residues of SEQ ID No: 22, and the beta chain variable domain comprises amino acid residues of SEQ ID No: 23) or an AFP TCR in which in which the alpha chain comprises amino acid residues of SEQ ID No: 41, and the beta chain variable domain comprises amino acid residues of SEQ ID No: 23 or SEQ ID NO: 42.

Accordingly such mutated heterologous TCRs can comprise an
a. αCDR1 having the sequence DRGSQA, SEQ ID NO:32
b. αCDR2 having the sequence AVNSDSSYALNF, SEQ ID NO:33
c. αCDR2 having the sequence AVNSDSGVALNF, SEQ ID NO:34
d. αCDR1 having the sequence DRGSQA, SEQ ID NO:32 and αCDR2 having the sequence AVNSDSGVALNF, SEQ ID NO:34
e. αCDR2 having the sequence AVNSQSGYALNF, SEQ ID NO: 35
f. αCDR2 having the sequence AVNSQSGYSLNF, SEQ ID NO: 36
g. αCDR2 having the sequence AVNSQSSYALNF, SEQ ID NO: 40
h. αCDR1 having the sequence DRGSQA, SEQ ID NO:32 and αCDR2 having the sequence AVNSQSGYALNF, SEQ ID NO: 35
i. αCDR2 having the sequence AVNSQSGVALNF, SEQ ID NO: 36
j. αCDR2 having the sequence AVNSQNGYALNF, SEQ ID NO: 37
k. αCDR1 having the sequence DRGSFS, SEQ ID NO: 38
l. αCDR1 having the sequence DRGSYS, SEQ ID NO: 39
m. αCDR1 having the sequence DRGSYS, SEQ ID NO: 39 and αCDR2 having the sequence AVNSDSSYALNF SEQ ID NO: 33,
n. αCDR1 having the sequence DRGSYS, SEQ ID NO: 39 and αCDR2 having the sequence AVNSDSSYALNF SEQ ID NO: 33,
o. αCDR1 having the sequence DRGSYS, SEQ ID NO: 39 and αCDR2 having the sequence AVNSQSGYALNF, SEQ ID NO: 35

According to the invention the heterologous TCR, or heterologous mutated TCR, can comprise an alpha chain variable domain that includes a mutation in one or more of the amino acids corresponding to: 31Q, 32S, 94D, 95S, 96G, 97Y, and 98A, with reference to the numbering shown in SEQ ID No: 22. For example, the alpha chain variable domain may have one or more of the following mutations: Q31F/Y, S32A, D94Q, S95N, G96S, Y97V, A98S, according to the numbering shown in SEQ ID No: 22.

According to the invention the heterologous TCR can comprise a TCR alpha chain variable domain of SEQ ID NO: 22 but that includes a mutation in one or more of the amino acids corresponding to: 31Q, 32S, 94D, 95S, 96G, 97Y, and 98A, for example one or more of Q31F/Y, S32A, D94Q, S95N, G96S, Y97V, A98S, according to the numbering shown in SEQ ID No: 22, and/or a beta chain variable domain comprising D1 to 1112 of SEQ ID NO: 23 or SEQ ID NO: 42.

According to the invention the heterologous TCR can comprise a TCR alpha chain variable domain and a TCR beta chain variable domain, wherein:
(i) the alpha chain variable domain comprises CDRs having the sequences
DRGSQA (αCDR1), SEQ ID NO:32 or amino acids 27-32 of SEQ ID NO:41,
IYSNGD (αCDR2), SEQ ID NO:27 or amino acids 50-55 of SEQ ID NO:22, and
AVNSQSGYALNF (αCDR3), SEQ ID NO:35 or amino acids 90-101 of SEQ ID NO:41, and
(ii) the beta chain variable domain comprises CDRs having the sequences
SGDLS (βCDR1), SEQ ID NO:29 or amino acids 27-31 of SEQ ID NO:23,
YYNGEE (βCDR2), SEQ ID NO:30 or amino acids 49-54 of SEQ ID NO:23, and
ASSLGGESEQY (βCDR3), SEQ ID NO:31 or amino acids 92-102 of SEQ ID NO:23.

According to the invention the heterologous TCR may comprise a TCR in which the alpha chain variable domain comprises amino acid residues 1-112 of SEQ ID No: 41, and the beta chain variable domain comprises amino acid residues 1-112 of SEQ ID No: 33 or SEQ ID NO:42.

According to the invention the heterologous TCR may comprise a TCR in which the alpha chain comprises amino acid residues of SEQ ID No: 41, and the beta chain variable domain comprises amino acid residues 1-112 of SEQ ID No: 33 or SEQ ID NO:42.

### TCR Variants

Amino acid and nucleotide sequence identity is generally defined with reference to the algorithm GAP (GCG Wisconsin Package™, Accelrys, San Diego CA). GAP uses the Needleman & Wunsch algorithm (J. Mol. Biol. (48): 444-453 (1970)) to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 2444-2448), or the Smith-Waterman algorithm (Smith and Waterman (1981) J. Mol Biol. 147: 195-197), generally employing default parameters.

Particular amino acid sequence variants may differ from a reference sequence by insertion, addition, substitution or deletion of 1 amino acid, 2, 3, 4, 5-10, 10-20 or 20-30 amino acids. In some embodiments, a variant sequence may comprise the reference sequence with 1, 2, 3, 4, 5, 6, 7, 8, 9, 10 or more residues inserted, deleted or substituted. For example, up to up to 20, up to 30 or up to 40 residues may be inserted, deleted or substituted.

In some preferred embodiments, a variant may differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. Conservative substitutions may, for example, be between amino acids within the following groups:
alanine and glycine;
glutamic acid, aspartic acid, glutamine, and asparagine
arginine and lysine;
asparagine, glutamine, glutamic acid and aspartic acid
isoleucine, leucine and valine;
phenylalanine, tyrosine and tryptophan
serine, threonine, and cysteine.

### The CD8α Co-Receptor

According to the present invention, the modified induced pluripotent stem cell iPSC or haemogenic lineage cell comprising at least one heterologous nucleic acid sequence encoding a heterologous TCR integrated into a locus in the cell genome and/or expressing or presenting a heterologous TCR according to the invention may further express or present a heterologous co-receptor (e.g., the cell is transduced with or engineered to comprise for example by gene knock-in, a nucleic acid encoding the co-receptor, e.g. CD8 co-receptor). The heterologous co-receptor may be a CD8 co-receptor. The CD8 co-receptor may comprise a dimer or pair of CD8 chains which comprises a CD8-α and CD8-β chain or a CD8-α and CD8-α chain. Preferably, the CD8 co-receptor is a CD8αα co-receptor comprising a CD8-α and CD8-α chain. A CD8α co-receptor may comprise the amino acid sequence of at least 80% identity to SEQ ID NO: 3, SEQ ID NO: 3 or a variant thereof. The CD8α co-receptor may be a homodimer.

Preferably the CD8 co-receptor binds to class 1 MHCs and potentiates TCR signalling. According to the invention the CD8 co-receptor may comprise the reference amino acid sequence of SEQ ID NO: 3 or may be a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence SEQ ID NO: 3. The CD8 co-receptor may be encoded by the reference nucleotide sequence of SEQ ID NO: 4 or may be a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence SEQ ID NO: 4.

According to the invention the heterologous CD8 co-receptor may comprise a CD8 co-receptor in which, in the Ig like V-type domain comprises CDRs having the sequence;
(i) VLLSNPTSG, CDR1, SEQ ID NO: 17, or amino acids 45-53 of SEQ ID NO: 3,
(ii) YLSQNKPK, CDR2, SEQ ID NO: 18 or amino acids 72-79 of SEQ ID NO: 3,
(iii) LSNSIM, CDR3, SEQ ID NO: 19 or amino acids 80-117 of SEQ ID NO: 3, or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto respectively.

According to the invention the heterologous CD8 co-receptor may comprise a CD8 co-receptor which comprises or in which, in the Ig like V-type domain comprises, residues 22-135 of the amino acid sequence of SEQ ID No: 3, or an amino acid sequence in which amino acid residues 22-44, 54-71, 80-117, 124-135 thereof have at least 70%, 75%, 80%, 85%, 90% or 95% identity to the sequence of amino acid residues 22-44, 54-71, 80-117, 124-135, CDR 1, CDR 2, CDR 3, respectively of SEQ ID No: 3 and in which amino acid residues 45-53, 72-79 and 118-123 have at least 70%, 75%, 80%, 85%, 90% or 95% identity to the sequence of amino acid residues 45-53, 72-79 and 118-123 respectively of SEQ ID No: 3.

According to the invention the CD8 co-receptor may comprise a CD8 co-receptor in which, or in which in the Ig like V-type domain, the sequence of:
(i) amino acid residues 22-44 thereof may have (a) at least 70%, 75%, 80%, 85%, 90% or 95% identity to the sequence of amino acid residues 22-44 of SEQ ID NO:3 or (b) may have one, two or three amino acid residues inserted or deleted relative to residues 22-44 of SEQ ID NO:3,
(ii) amino acid residues 45-53 is VLLSNPTSG, SEQ ID NO:17, CDR1, or amino acids 45-53 of SEQ ID NO:3,
(iii) amino acid residues 54-71 thereof may have (a) at least 70%, 75%, 80%, 85%, 90% or 95% identity to the sequence of amino acid residues 54-71 of SEQ ID NO:3 or (b) may have one, two or three amino acid residues inserted or deleted relative to the sequence of amino acid residues 54-71 of SEQ ID NO:3,
(iv) amino acid residues 72-79 may be YLSQNKPK, CDR2, SEQ ID NO:18 or amino acids 72-79 of SEQ ID NO:3,
(v) amino acid residues 80-117 thereof may have at least 70%, 75%, 80%, 85%, 90% or 95% identity to the sequence of amino acid residues 80-117 of SEQ ID NO:3 or may have one, two or three insertions, deletions or substitutions relative to the sequence of amino acid residues 80-117 of SEQ ID NO:3;
(vi) amino acids 118-123 may be LSNSIM, CDR3, SEQ ID NO:19 or amino acids 80-117 of SEQ ID NO:3,
(vii) amino acid residues 124-135 thereof may have at least 70%, 75%, 80%, 85%, 90% or 95% identity to the sequence of amino acid residues 124-135 of SEQ ID NO:3 or may have one, two or three insertions, deletions or substitutions relative to the sequence of amino acid residues 124-135 of SEQ ID NO:3.

The modified induced pluripotent stem cells iPSC or haemogenic lineage cells that comprise a nucleic acid encoding the heterologous CD8 co-receptor and/or which express heterologous CD8 co-receptor may demonstrate improved affinity and/or avidity and/or improved T-cell activation, as determinable by the assays disclosed herein, towards or on stimulation by antigenic peptide, tumour or cancer antigen optionally when presented on HLA relative to modified cells (e.g. induced pluripotent stem cells iPSC or haemogenic lineage cells) that do not express heterologous CD8 co-receptor. The heterologous CD8 of modified cells may interact or bind specifically to an MHC, the MHC may be class I or class II, preferably class I major histocompatibility complex (MHC), HLA-I molecule or with the MHC class I HLA-A/B2M dimer, preferably the CD8-α interacts with the α₃ portion of the Class I MHC (between residues 223 and 229), preferably via the IgV-like domain of CD8. Accordingly the heterologous CD8 improves TCR binding of the cells to the HLA and/or antigenic peptide bound or presented by HLA pMHCI or pHLA, optionally on the surface of antigen presenting cell, dendritic cell and/or tumour or cancer cell, tumour or cancer tissue compared to cells lacking the heterologous CD8. Accordingly the heterologous CD8 can improve or increase the off-rate (k_{off}) of the cell (TCR)/peptide-major histocompatibility complex class I (pMHCI) interaction of the modified induced pluripotent stem cells iPSC or haemogenic lineage cells, and hence its half-life, optionally on the surface of antigen presenting cell, dendritic cell and/or tumour or cancer cell, or tumour or cancer tissue compared to cells (iPSC or haemogenic lineage cells) lacking the heterologous CD8, and thereby may also provide improved ligation affinity and/or avidity. The heterologous CD8 can improve organizing the TCR on the stem cells iPSC or haemogenic lineage cells surface to enable cooperativity in pHLA binding and may provide improved therapeutic avidity. Accordingly, the heterologous CD8 co-receptor modified induced pluripotent stem cells iPSC or haemogenic lineage cells may bind or interact with LCK (lymphocyte-specific protein tyrosine kinase) in a zinc-dependent manner leading to activation of transcription factors like NFAT, NF-κB, and AP-1.

According to the invention the modified induced pluripotent stem cells iPSC or haemogenic lineage cells may have an improved or increased expression of CD40L, cytokine production, cytotoxic activity, induction of dendritic cell maturation or induction of dendritic cell cytokine production, optionally in response to cancer and/or tumour antigen or peptide antigen thereof optionally as presented by tumour of cancer cell or tissue, in comparison to pluripotent stem cell iPSC or haemogenic lineage cells lacking the heterologous CD8 co-receptor.

### Co-Stimulatory Ligand

According to the present invention, the modified pluripotent stem cell iPSC or haemogenic lineage cells, may further comprise (e.g. express or present) an exogenous or heterologous or recombinant co-stimulatory ligand (e.g., the cell is transduced with or engineered to comprise for example by gene knock-in, a nucleic acid encoding the co-stimulatory ligand) at least one co-stimulatory ligand, optionally one, two, three or four. The modified cell or cells, may co-express the heterologous TCR and the at least one exogenous co-stimulatory ligand. The interaction between the heterologous TCR and at the least one exogenous co-stimulatory ligand may provide a non-antigen-specific signal and activation of the cell. Co-stimulatory ligands include, but are not limited to, members of the tumour necrosis factor (TNF) superfamily, and immunoglobulin (Ig) superfamily ligands. TNF is a cytokine involved in systemic inflammation and stimulates the acute phase reaction. TNF superfamily members include, but are not limited to, nerve growth factor (NGF), CD40L (CD40L)/CD154, CD137L/4-1BBL, TNF-alpha, CD134L/OX40L/CD252, CD27L/CD70, Fas ligand (FasL), CD30L/CD153, tumour necrosis factor beta (TNFP)/lymphotoxin-alpha (LTa), Iymphotoxin-beta (TTb), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28. In certain embodiments, the at least one co-stimulatory ligand is selected from the group consisting of 4-1BBL, CD275, CD80, CD86, CD70, OX40L, CD48, TNFRSF14, and combinations thereof. The at least one exogenous or recombinant co-stimulatory ligand can be 4-1BBL or CD80, preferably, the at least one exogenous or recombinant co-stimulatory ligand is 4-1BBL. The modified pluripotent stem cell iPSC or haemogenic lineage cells may comprise two exogenous or recombinant co-stimulatory ligands, preferably the two exogenous or recombinant co-stimulatory ligands are 4-1BBL and CD80.

The modified cells may comprise an exogenous or a recombinant (e.g., the cell is transduced with or engineered to comprise for example by gene knock-in, a nucleic acid encoding) at least one construct which overcomes the immunosuppressive tumour microenvironment. Such constructs can be, but are not limited to, cyclic AMP phosphodiesterases and dominant-negative transforming growth factor beta (TGFbeta) receptor II. The modified stem cells iPSC or haemogenic lineage cells, modified T cell or a population of modified T cells may be engineered to release cytokines which have a positive effect on the cytolytic activity of said cells. Such cytokines include, but are not limited to interleukin-7, interleukin-15 and interleukin-21.

### Cell Function and Activation

The present invention provides a modified induced pluripotent stem cell iPSC or haemogenic lineage cell according to the invention, wherein the binding of the iPSC or haemogenic lineage cell and/or heterologous TCR to the antigen or peptide antigen, e.g. cancer and/or tumour antigen or peptide antigen thereof or a cancer and/or tumour cell or tissue expressing or presenting the cancer and/or tumour antigen or peptide antigen thereof as herein described, can induce activation and/or function of the iPSC or haemogenic lineage cell, optionally as determined by any one or more of;
(a) up-regulation of T cell activation markers, for example either CD69 and/or CD25 on CD3+ cells,
(b) up-regulation of cytokine production, for example any one or more of IFN gamma, IL-2 or Granzyme B,
(c) induced cell cytotoxic activity in the presence of the antigen or antigen peptide,
(d) ability to kill tumour cells presenting the antigen or antigen peptide,
(e) increased iPSC or haemogenic lineage cell secretion of cytokines and/or interferon,
(f) increased iPSC or haemogenic lineage cell proliferation, for example as judged by cell markers (for example as judged by Ki67 expression level),
(g) increased iPSC or haemogenic lineage cell antigen responsiveness,
(h) increased iPSC or haemogenic lineage cell target cell killing,
(i) increased iPSC or haemogenic lineage cell CD28 signalling,
(j) increased iPSC or haemogenic lineage cell ability to infiltrate tumour,
(k) increased ability of iPSC or haemogenic lineage cell to recognise and bind to dendritic cell presented antigen
(l) increased iPSC or haemogenic lineage cell ability to resist T-cell repressive factors as determined for example by a reduced repressive response to T-regs, Myeloid derived suppressor cells (MDSCs), PD-L1 protein expression, or serum cytokine levels selected from CCL3, IL8, IL1β, CXCL10, or sIL2Rα or levels of inhibitory receptors, selected from PD-1, CTLA-4, TIM-3, LAG-3, BTLA or TIGIT, or
(m) increase in level of interferon-γ, interleukin-6, interleukin-10, cytokine production, such as IL-2, TNF-α, IFN-γ and granzyme B by the iPSC or haemogenic lineage cell.

### TCR+ Cell Persistence

The present invention provides a modified induced pluripotent stem cell iPSC or haemogenic lineage cell according to the invention, which demonstrate improved TCR+ cell persistence in culture or subject samples for example, as determined by the measurement of the persistence of modified pluripotent stem cell iPSC or haemogenic lineage cells expressing or presenting a heterologous T-cell receptor (TCR) as herein described. Persistence of the infused engineered and modified pluripotent stem cell iPSC or haemogenic lineage cells is correlated with therapeutic effect and is also a long-term safety measure. Cell persistence can be determined by qPCR or flow cytometry (FCM). For example, the quantitation of TCR+(e.g. AFP TCR or MAGE-A4 TCR) cells by PCR of coding gene from DNA. Alternatively, quantification of T cell phenotype and activity associated with the heterologous TCR may be determined by a range of assays, for example:
Phenotype analysis for determination of T-cell lineages in cell product/culture or in the subject blood pre and post infusion.
Quantitation of the senescence and activation status of T-cells in cell product/culture or from subject samples
Quantitation of soluble factors reflecting in vivo function of TCR+ T cells,

### Nucleic Acid Construct or Vector

The present invention provides a nucleic acid construct or vector comprising a nucleic acid region encoding the heterologous TCR according to the invention and at least one homology region comprising a nucleic acid region homologous to a nucleic acid region at the locus in the cell genome for integration of the nucleic acid region encoding the heterologous TCR. Preferably the locus is as herein before described.

Preferably the locus is or is in a gene encoding a membrane protein or transmembrane protein, optionally a receptor protein, preferably a receptor protein tyrosine phosphatase (PTP), preferably CD45 or protein tyrosine phosphatase receptor type C or PTPRC.

Accordingly and preferably the locus is or is in the PTPRC (CD45) gene on chromosome 1, optionally wherein the locus is at exon 33 of the PTPRC (CD45) gene, optionally before the TAG stop codon or immediately adjacent to and/or before the TAG stop codon.

The nucleic acid encoding the heterologous TCR may comprise a coding sequence of a TCRα and/or TCRβ chain, for example as described herein, optionally with an intervening nucleic acid sequence encoding a peptide comprising an enzymatic cleavage site and/or nucleic acid sequence which mediates ribosome-skipping, preferably wherein the nucleic acid sequence encoding a peptide comprising an enzymatic cleavage site encodes a furin cleavage site, preferably RAKR, preferably wherein the nucleic acid sequence which mediates ribosome-skipping is a ribosome skip sequence, preferably T2A and/or P2A skip sequence.

According to the invention the construct or vector can comprise a left hand and/or a right hand homology region each homologous to a nucleic acid region at the locus in the cell genome for integration of the nucleic acid region encoding the heterologous TCR, optionally which flank opposite sides of the integration site.

Accordingly the construct or vector may further comprise any one or more of:
(a) a recombination target sequence, preferably loxP (locus of X-over P1) sequence,
(b) an expressible selection marker sequence, preferably an antibiotic resistance gene, optionally a neomycin resistance gene, preferably constitutively expressed from a promoter for example from an EF1A promoter.

According to the invention the construct or vector may comprise a nucleotide sequence encoding the heterologous TCR comprising the sequence SEQ ID No: 45, homology regions comprising the sequences SEQ ID No: 43 and SEQ ID No: 44, and optionally a recombination target sequence comprising SEQ ID No: 48 and/or an expressible selection marker sequence comprising SEQ ID No: 47 and SEQ ID No: 49.

The present invention further provides a process of producing a modified induced pluripotent stem cell iPSC or haemogenic lineage cell according to the invention comprising introducing the nucleic acid construct or vector according to the invention into an unmodified induced pluripotent stem cell iPSC or haemogenic lineage cell, optionally as herein described, under conditions to permit integration of the nucleic acid sequence encoding a heterologous T-cell receptor (TCR) at or into a locus in the cell genome and optionally isolating the modified induced pluripotent stem cell iPSC or haemogenic lineage cell. The process may be achieved using known genome editing methodologies such as those which employ AAV, Transcription activator-like effector nucleases (TALEN), CRISPR, Zinc finger nucleases, or engineered meganucleases

### Treatment

The present invention further provides a pharmaceutical composition comprising the modified induced pluripotent stem cell iPSC or haemogenic lineage cell according to the invention and a pharmaceutically acceptable carrier.

The present invention further provides a modified induced pluripotent stem cell iPSC or haemogenic lineage cell or pharmaceutical composition according to the invention, for use in therapy and/or medicine.

The invention also provides any one or more of the following medical uses or methods of treatment comprising:
(a) a modified induced pluripotent stem cell iPSC or haemogenic lineage cell or pharmaceutical composition according to the invention, for use in treatment, prevention or delaying the progression of cancer and/or tumour in an individual or subject optionally wherein the treatment is cancer immunotherapy therapy and/or adoptive T cell therapy, optionally allogenic adoptive T cell therapy,
(b) use of a modified induced pluripotent stem cell iPSC or haemogenic lineage cell or pharmaceutical composition according to the invention, in the manufacture of a medicament for the treatment of cancer and/or tumour in an individual or subject, optionally wherein the treatment is cancer immunotherapy therapy and/or adoptive T cell therapy, optionally allogenic adoptive T cell therapy,
(c) a method of treating, preventing or delaying the progression of cancer and/or tumour in an individual or subject comprising administering to the individual the modified induced pluripotent stem cell iPSC or haemogenic lineage cell or pharmaceutical composition according to the invention, optionally wherein the treatment is cancer immunotherapy therapy and/or adoptive T cell therapy, optionally allogenic adoptive T cell therapy.

According to the present invention the cancer and/or tumour, which may be a solid tumour, may be selected from; lung cancer, non-small cell lung cancer (NSCLC), metastatic or advanced NSCLC, squamous NSCLC, adenocarcinoma NSCLC, adenosquamous NSCLC, large cell NSCLC, ovarian cancer, gastric cancer, urothelial cancer, esophageal cancer, esophagogastric junction cancer (EGJ), melanoma, bladder cancer, head and neck cancer, head and neck squamous cell carcinoma (HNSCC), cancer of the oral cavity, cancer of the oropharynx, cancer of the hypopharynx, cancer of the throat, cancer of the larynx, cancer of the tonsil, cancer of the tongue, cancer of the soft palate, cancer of the pharynx, synovial sarcoma, myxoid round cell liposarcoma (MRCLS): optionally wherein the cancer or tumour express a MAGE or AFP protein, peptide, antigen or peptide antigen thereof as described herein, optionally MAGE-A4 protein, peptide, antigen or peptide antigen thereof as described herein.

According to the present invention the cancer may be selected from any one or more of; breast cancer, metastatic breast cancer, liver cancer, renal cell carcinoma, synovial sarcoma, urothelial cancer or tumour, pancreatic cancer, colorectal cancer, metastatic stomach cancer, metastatic gastric cancer, metastatic liver cancer, metastatic ovarian cancer, metastatic pancreatic cancer, metastatic colorectal cancer, metastatic lung cancer, colorectal carcinoma or adenocarcinoma, lung carcinoma or adenocarcinoma, pancreatic carcinoma or adenocarcinoma, mucinous adenoma, ductal carcinoma of the pancreas, hematological malignancy: optionally wherein the cancer or tumour express a MAGE or AFP protein, peptide, antigen or peptide antigen thereof as described herein, optionally MAGE-A4 protein, peptide, antigen or peptide antigen thereof as described herein.

According to the invention the cancer and/or tumour can be liver cancer, or can be liver cancer selected from any of; cholangiocarcinoma, liver angiosarcoma, hepatoblastoma, hepatocellular carcinoma (HCC), optionally wherein the cancer is not amenable to transplant or resection, preferably the cancer is hepatocellular carcinoma (HCC); additionally the liver cancer may be coincident with any one or more of; diabetes, obesity, hepatitis B, hepatitis C, cirrhosis: optionally wherein the cancer or tumour express a MAGE or AFP protein, peptide, antigen or peptide antigen thereof as described herein, optionally MAGE-A4 protein, peptide, antigen or peptide antigen thereof as described herein.

According to the present invention the cancer can be relapsed cancer or refractory cancer or recurrent cancer or locally recurrent cancer or metastatic cancer, non-resectable cancer or locally confined, cancer with no surgical or radiotherapy option or inoperable cancer, or any combination thereof. According to the invention the cancer and/or tumour may be a solid tumour.

The invention also provides a modified induced pluripotent stem cell iPSC or haemogenic lineage cell or pharmaceutical composition according to the invention, for use in a method of,
(a) up-regulation of T cell activation,
(b) up-regulation of cytokine production, for example any one or more of IFN gamma, IL-2 or Granzyme B,
(c) induction of cell cytotoxic activity in the presence of the antigen or antigen peptide, or
(d) inducing the killing of tumour cells presenting the antigen or antigen peptide; optionally in a subject with cancer and/or tumour, optionally wherein the cancer and/or tumour express a cancer and/or tumour antigen or peptide antigen thereof as described herein, preferably a MAGE or AFP protein, peptide, antigen or peptide antigen thereof as described herein.

### Combinations

The present invention further provides a modified induced pluripotent stem cell iPSC or haemogenic lineage cell or pharmaceutical composition according to the invention, for use according to the forgoing medical uses or methods of treatment, wherein the modified induced pluripotent stem cell iPSC or haemogenic lineage cell, or pharmaceutical composition is for use, or is used, in combination with one or more further therapeutic agent optionally administered or for administration separately, sequentially or simultaneously.

Accordingly, the one or more further therapeutic agent can comprise, chemotherapy agent, hormonal therapy agent, a targeted drug, or immunotherapy. Accordingly the chemotherapy may comprise any one or more of, gemcitabine, docetaxel, pemetrexed, topotecan, irinotecan, etoposide, vinorelbine Lipoplatin, Cisplatin, Carboplatin, Oxaliplatin, Nedaplatin, Triplatin tetranitrate, Phenanthriplatin, Satraplatin, Picoplatin, methotrexate, capecitabine, taxane, anthracycline, paclitaxel, docetaxel, paclitaxel protein bound particles, doxorubicine, epirubicine, 5-fluorouracil, cyclophosphamide, vincristine, etoposide. Additionally, or alternatively the chemotherapy may comprise one or more chemotherapeutic agent selected from, FEC: 5-fluorouracil, epirubicine, cyclophosphamide; FAC: 5-fluorouracil, doxorubicine, cyclophosphamide; AC: doxorubicine, cyclophosphamide; EC: epirubicine, cyclophosphamide. Additionally or alternatively the one or more further therapeutic agent can comprise any one or more of Sorafenib, a PD1 or PD-L1 antagonist or inhibitor, Regorafenib, Cabozantinib, Sunitinib Brivanib, Everolimus, Tivantinib, Linifanibfrom Sorafenib, a PD1 or PD-L1 antagonist or inhibitor, Regorafenib, Cabozantinib, Sunitinib, Brivanib, Everolimus, Tivantinib, Linifanib, or a tyrosine kinase inhibitor or epidermal growth factor receptor (EGFR) inhibitor, such as crizotinib, erlotinib, gefitinib and afatinib, an immune checkpoint inhibitor, such as pembrolizumab or nivolumab, or monoclonal antibody against nuclear factor kappa-B ligand, such as denosumab, or an Epidermal Growth Factor Receptor Antagonist antibody, optionally Cetuximab.

As used herein the term “modified induced pluripotent stem cell iPSC or haemogenic lineage cell” may also include modified induced pluripotent stem cells iPSCs or haemogenic lineage cells or modified population of induced pluripotent stem cells iPSCs or population haemogenic lineage cells, (wherein the term modified also applies to both IPSC(s) and haemogenic lineage cell(s)).

### Kit

The present invention further provides a kit comprising,
(a) the modified induced pluripotent stem cell iPSC or haemogenic lineage cell or pharmaceutical composition according to the invention, and a package insert comprising instructions for use thereof for treating, preventing or delaying the progression of cancer and/or tumour in an individual or subject, or
(b) the modified induced pluripotent stem cell iPSC or haemogenic lineage cell or pharmaceutical composition according to the invention, and a package insert comprising instructions for use thereof for treating, preventing or delaying the progression of cancer and/or tumour in an individual or subject, in combination with one or more further therapeutic agent, optionally as herein described, optionally administered or for administration separately, sequentially or simultaneously

The invention will be further described by reference to the following figures and examples.

### FIGURES

FIG. 1—PTPRC splice variants. The PTPRC gene is located on chromosome 1 and contains 33 exons. Multiple splice variants are produced from this gene via alternative splicing. The splice variants are a result of splicing at exons 4-6. Each transcript contains the same 3′ exon structure. Figure adapted from Tchilian and Beverley 2006. Trends in Immunology.

FIG. 2—ADP A2M4 knock-in strategy—The targeting strategy was deigned to knock ADP A2M4 SPEAR into PTPRC exon 33. Editing was performed with AAV. The homology arms in the rAAV repair template correspond the 500 bp of genomic sequence upstream and downstream of the stop codon. Editing creates a multicistronic fusion between PTPRC and ADP A2M4 with each polypeptide coding sequence separated by a furin cleavage site (RAKR) and 2A skip peptide (PTPRC_FuT2A_TCRα_FuP2A_TCRβ). This design permits the expression of all 3 polypeptides from a single edited allele. Knock-in into Exon 33 also allows the expression of A2M4 across multiple PTPRC splice variants. The resistance cassette contains a LOXP flanked NEO resistance gene constitutively expressed from the EF1A promoter

FIG. 3—Vector map pAAV MCS_PTPRC_FuT2A_A2M4_LOXP_NEO. The targeting cassette containing the homology arms (LHA and RHA), FuT2A_A2M4 TCR and NEO resistance cassette was cloned between the NotI restriction sites in the pAAV MCS vector (Agilent). With the exception of the vector backbone, each fragment was amplified by PCR and fragments cloned into the vector by Gibson assembly.

FIG. 4—PCR screening strategy. Edited clones were isolated by limiting dilution in 96 well plates. A sample of gDNA was isolated from each clone using QuickExtract™. Clones were screened by PCR with primers outside the homology arm regions and within the transgene cassette. The LHA boundary PCR was performed using primers PTPRC-5int-FWD and PTPRC-5int-REV. The RHA boundary PCR was performed with primers PTPRC-3int-FWD and PTPRC-3int-REV. PCR products were sequenced. Three clones were progressed for differentiation. ChiPSC31 PTPRC_A2M4 clone 2, ADAPiJ001_J PTPRC_A2M4 clone 3D10 and ADAPiJ001_J PTPRC_A2M4 clone 3G5.

FIG. 5—Expression of ADP A2M4 in iT cells can be induced via lentiviral transduction of CD7+CD5+ progenitors. The iPSC line ADAPi001_J was differentiated and transduced with lentivirus express containing the ADP A2M4 T cell at Stage 4 (CD7+CD5+ progenitor). Transduced cells were progressed through the differentiation protocol. Cells were phenotyped by FACS and ADP A2M4 expression measured by staining with anti-Vα24 antibody and A2M4 MAGE-4 dextramer.

FIG. 6—Phenotypes of iT cells differentiated from edited iPSC clones (Stage 5): (A) iT cells differentiated from (A) ChiPSC31 PTPRC_A2M4 clone 2 (single targeted allele), (B) iT cells differentiated from ADAPi001_J PTPRC_A2M4 clone 3D10 (both alleles targeted) and (C) iT cells differentiated from ADAPi001_J PTPRC_A2M4 clone 3G5 (single targeted allele). Differentiation is unaffected by editing. Edited clones express the ADP A2M4 SPEAR. Cells were phenotyped by FACS and A2M4 expression measured by staining with anti-Vα24 antibodies and ADP A2M4 MAGE-4 dextramer. Knock-in at a single allele is sufficient to promote ADP A2M4 expression.

FIG. 7—iT cells differentiated from edited iPSC clones exhibit specific and potent cytolytic effector function: iT cell cytolytic function was assessed with the KILR assay. T2 cells were pulsed with decreasing concentrations (10⁻⁵ M-10⁻¹¹ M) of the MAGE-A4 derived peptide GVYDGREHTV and incubated with iT cells differentiated from ADAPi001_J (ADAP001_J WT NTD (non-transduced)), ADAPi001_J derived, iT cells transduced with ADP A2M4 lentivirus (ADAPi001_J WT A2M4 TD) and iT cells differentiated from edited ADAPi001_J PTPRC_A2M4 clone 3G10. Cytolytic function was compared to ADP A2M4 transduced and non-transduced PBL isolated from healthy donors.

FIG. 8—iT cells up-regulate the activation markers CD69 and CD25 when incubated with antigen positive tumour cells lines: iT cells differentiated from the ADAPi001_J iPSC line (NTD non-transduced), ADAPi001_J derived iT cells transduced with ADP A2M4 lentivirus (TD) and iT cells differentiated from edited ADAPi001_J PTPRC_A2M4 clone 3G10 were co-cultured (24 hrs) with the tumour lines A375 (HLA-A*02 positive and MAGE-A4 positive) and COLO205 (HLA-A*02 positive and MAGE-A4 negative) in the presence or absence of the MAGE-A4 peptide GVYDGREHTV (0.5 μg). Following co-culture, up-regulation in expression of the T cell activation markers CD69 and CD25 was determined by flow cytometry. The expression of activation markers was compared to ADP A2M4 transduced and non-transduced PBL isolated from healthy donors.

FIG. 9—Sequence validation of the INDELs present within the two RAG1 alleles (Exon2) in ADAPi001_J_7D10_RAG1^{-/-}. One allele (top) contains an 11 bp deletion and the second (bottom) contains a 16 bp deletion. Both INDELs are predicted to inactivate RAG1. Clone 7D10 ADAPi001_J_7 D10_RAG1^{-/-} was used to knock-in the A2M4 TCR into PTPRC (Exon33)

FIG. 10—RAG1^{-/-} iT cells do not express CD3 and TCR. The expression of cell surface markers CD45, CD4, CD8α, CD8β, CD56, CD3, TCR chain Vα-24 (binds to A2M4-TCR) and αβTCR were measured by FACS. Phenotypes of differentiated iT-cells (A) Wild Type ADAPi001_J (B) A2M4 lentivirus transduced wild type ADAPi001_J (C) ADAPi001_J_7 D10_RAG1^{-/-} (D) A2M4 lentivirus transduced ADAPi001_J_7 D10_RAG1^{-/-}.

FIG. 11—Monoallelic knock-in of the A2M4 TCR into PTPRC rescues CD3 and TCR expression RAG1^{-/-} iT-cells. A single copy of the A2M4 TCR was knocked into PTPRC (Exon33) in the RAG1^{-/-} iPSC clone ADAPi001_J_7 D10. Three clones were isolated and differentiated into iT cells The expression of cell surface markers CD45, CD4, CD8α, CD8β, CD56, CD3, TCR chain Vα-24 (binds to A2M4-TCR) and αβTCR were measured by FACS. (A) ADAPi001_J_1B8_RAG1^{-/-}_PTPRC^{A2M4/WT} (B) ADAPi001_J_2D4_RAG1^{-/-}_PTPRC^{A2M4/WT} (C) ADAPi001_J_2E7_RAG1^{-/-}_ PTPRC^{A2M4/WT}.

FIG. 12—iT-cells differentiated from a RAG1^{-/-} iPSC clone can kill antigen positive tumour cell lines following transduction with a lentivirus encoding the A2M4 TCR. MAGE-A4 peptide (GVYDGREHTV) peptide and non-pulsed GFP^{+ve} A375 (MAGE-A4^{+ve}, HLA-A2*02^{+ve}) cells were co-cultured with non-transduced or A2M4 transduced iT-cells differentiated from wild type ADPAi001_J or ADPAi001_J_c7D10_RAG1^{-/-}. A decrease in Green object counts (GFP) represents death of GFP^{+ve} A375 tumour cell lines.

FIG. 13—iT-cells differentiated from RAG1^{-/-} PTPRC^{A2M4/WT} iPSC clones can kill antigen positive tumour cell. MAGE-A4 peptide (GVYDGREHTV) pulsed and non-pulsed GFP^{+ve} A375 (MAGE-A4^{+ve}, HLA-A2*02^{+ve}) cells were co-cultured iT-cells differentiated from the iPSC clones ADAPi001_J_c1B8_RAG1^{-/-}_ PTPRC^{A2M4/WT}, ADPAi001_J_c2D7_RAG PTPRC^{A2M4/WT} or ADPAi001_J_c2E4_RAG1^{-/-} PTPRC^{A2M4/WT}. A decrease in Green object counts (GFP) represents death of GFP^{+ve} A375 tumour cell lines.

### EXAMPLES

### Introduction

We describe here the generation of iT-cells expressing a defined heterologous recombinant αβTCR. In a first set of experiments, starting with a GMP compliant hiPSC source, we knocked-in the recombinant heterologous T-cell receptor ADP A2M4 that recognises the MAGE-A4 peptide (GVYDGREHTV) presented by HLA-A*02. We show that ADP A2M4 iT-cells derived from engineered iPSCs specifically express the ADP A2M4 TCR as measured by anti-TCRVα24 and dextramer staining. Edited ADP A2M4 iT-cells up regulate activation markers including CD25 and CD69 when incubated with HLA-A*02 expressing tumour lines that express the cognate antigen and exhibit potent antigen dependent killing of these lines. In a second set of experiments, we knocked-in the ADP A2M4 TCR into a RAG1 null iPSC line ADAPi001_J. The following examples represents the development of an allogeneic hiPSC derived platform, with limited genome editing, that permits the production of ADP A2M4 TCR iT-cells of therapeutic value and activity.

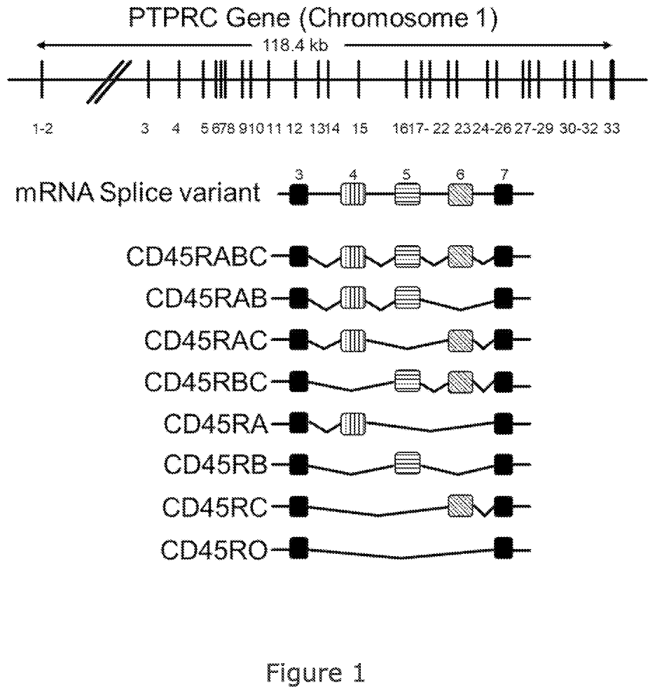

ADP A2M4 TCR is stably introduced into the iT-cells by knock-in at the PTPRC locus. PTPRC encodes a transmembrane protein tyrosine phosphatase (CD45) that is an important regulator of TCR signalling. The PTPRC gene contains 33 exons and encodes multiple isoforms (CD45RABC, CD45R0, CD45RAB, CD45RAC, CD45RBC, CD45RA, CD45RB and CD45RC) which are generated by alternative splicing of exons 4-6 (FIG. 1). The expression of CD45 isoforms is altered during T cell development, activation and differentiation. For example, CD45RA is primarily expressed on naïve T cell subsets whereas CD45RO is primarily expressed on memory T cell subsets.

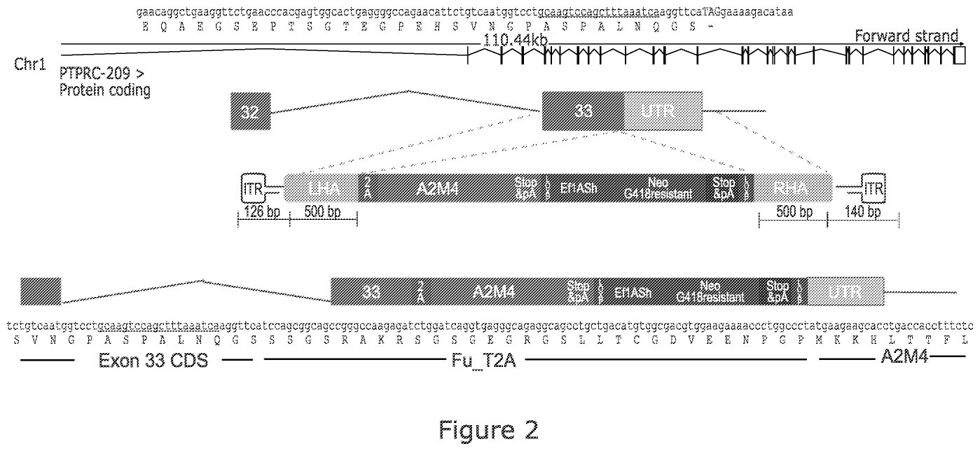

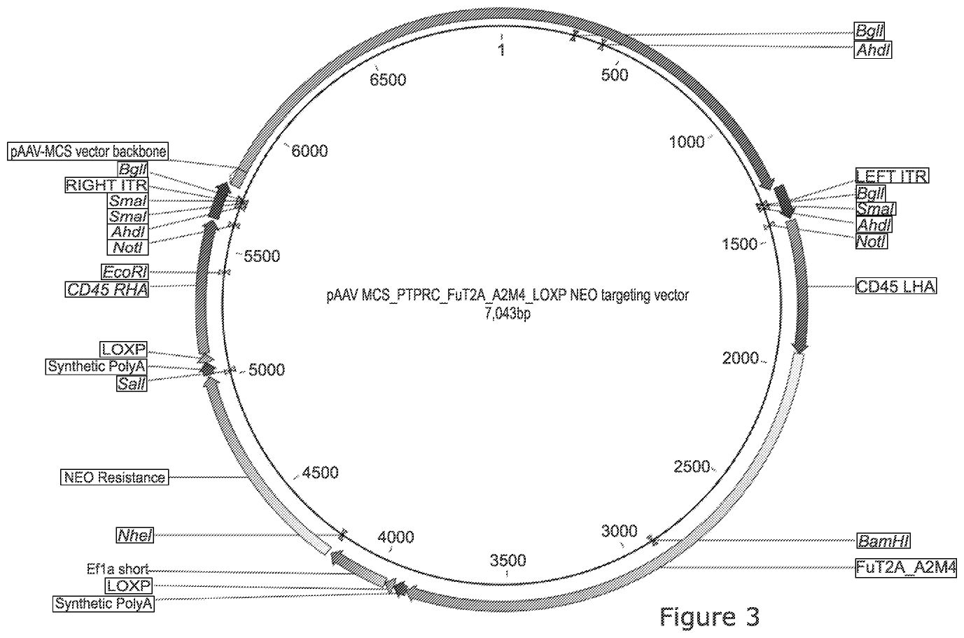

The PTPRC isoforms all share the same 3′ exon structure and thus modification of this region will be shared between all of the PTRPC isoforms. An rAAV targeting plasmid was generated to enable the knock-in of the ADP A2M4 TCR into PTPRC exon 33 immediately before the TAG stop codon. The construct was deigned to generate a multicistronic fusion gene between PTPRC and ADP A2M4 SPEAR. This permits the regulated transcription and expression of PTPRC isoforms and ADP A2M4 from the endogenous PTPRC promoter. A furin cleavage site combined with T2A or P2A skip sequence peptide separates the PTPRC and ADP A2M4 TCRα and A2M4 TCRβ coding sequences (FIGS. 2 and 3). This allows the production of multiple polypeptides from a single mRNA. Cleavage by the furin protease in the endoplasmic reticulum will remove the skip sequence peptides allowing the expression of nascent protein sequences.

Production of iPSC Cell Lines

The iPSC lines (ADAPi001) were created via re-programming of CD34+ progenitors isolated from umbilical cord blood using the pEB-C5 and pEB-TG episomal plasmids [Chou, B. K., et al., Efficient human iPS cell derivation by a non-integrating plasmid from blood cells with unique epigenetic and gene expression signatures. Cell Res, 2011. 21(3): p. 518-29]. All ADAPi001 clones are traceable and produced under GMP conditions. Nine ADAPi001 hiPSC clones were characterised with a small working cell bank of early passage number (p10) produced for each clone. Genomic stability was assessed via cyto-SNP analysis, karyotyping and WGS (whole genome sequencing). Pluripotency was determined with IHC (immunohistochemistry), flow cytometry and Pluritest [Muller, F. J., et al., A bioinformatic assay for pluripotency in human cells. Nat Methods, 2011. 8(4): p. 315-7]. Differentiation of iT-cells from ADAPi001 iPSC clones was also confirmed.

iPSC Cell Culture

The iPSC lines ChiPSC31 (Takara) and ADAPi001_J were used for editing experiments. The ChiPSC31 iPSC line was cultured on the COATI matrix with DEF-CS 500 culture media (Takara). The ADAPi001_J iPSC line was cultured on the Synthemax™ II-SC Substrate (Corning) with mTeSR™ Plus culture media (STEMCELL Technologies). Both iPSC lines were adapted for enzymatic dissociation with TrypLE™ Select (ThermoFisher) and passaged every 4-5 days. iPSC cultures were maintained in a humidified 37° C., 5% O₂, 5% CO₂ incubator. The expression of pluripotency markers (POU5F1, NANOG, TRA-160 and SOX2) and absence of the differentiation marker SSEA-1 was routinely monitored by FACS analysis.

### Generation of the AAV Targeting Vector.

The pAAV-MCS (Agilent) plasmid was digested with NotI (NEB) to remove the sequences between the left and right inverted terminal repeat (ITR) regions. The vector backbone was purified by agarose gel extraction. The left and right homology arms (LHA, SEQ ID No: 43 and RHA, SEQ ID No: 44), FuTA_A2M4 transgene cassette, SEQ ID No: 45, and LOPX, SEQ ID No: 48, flanked EF1A_Neo selection cassette SEQ ID No: 47 and SEQ ID No: 49 were amplified by PCR. Each primer contained overlapping sequence to allow generation of the targeting vector by Gibson assembly. The left and right homology arm sequences (500 bp) were amplified from genomic DNA (gDNA) isolated from the iPSC line ADAPi001_J. The LHA arm corresponds to nucleotides 198755679 to 198756178 on and RHA corresponds to nucleotides 198756182 to 198756681 Chromosome 1 (Genome build GRCh38 Ensembl release 99—January 2020). The ADP A2M4 TCR transgene contains the coding sequence of the TCRα and TCR chains with a furin cleavage site (RAKR) and P2A skip sequence between two TCR chains. The LOXP flanked selection cassette was synthetically synthesised (GeneArt). The pAAV_MCS_PTPRC_FuT2A_A2M4_LOXPNEO plasmid map is shown in FIG. 3.

### AAV Production

The packaging plasmids pHelper (Agilent 240071) and AAV-3 Rep-Cap Plasmid (Cell Biolabs VPK-423) used for rAAV production were grown in NEB® 5-alpha Competent E. coli. The AAV targeting plasmid containing the homology arms, FuT2A_A2M4 and NEO selection cassette were grown in NEB® Stable Competent E. coli. All plasmid DNA was purified using Plasmid Plus Giga Kit (Qiagen) or EndoFree Plasmid Giga Kit (Qiagen). The ITR integrity in the AAV transgene plasmid was confirmed with AhdI and BglI single restriction enzyme digest.

The 293AAV Cell Line (Cell Biolabs) used to produce rAAV was cultured with 10% (v/v) Heat Inactivated FBS (Life Technologies), DMEM, high glucose, GlutaMAX™ Supplement, pyruvate (Life technologies) Pen/Strep (Life Technologies) and Non Essential Amino Acids (Life Technologies). 293AAV were seeded in a 5-cell stack (Corning) at a density of 216×10⁶/5CS 24 hrs before transfection. 293AAV were transfected with the rAAV transgene plasmid, pHelper and AAV-3 Rep-Cap at a molar ratio of 2:1:1 using Turbofect transfection reagent (Thermofisher). A total 2.6 mg plasmid DNA was used for each transfection. Cells were cultured for 72 hrs before harvesting and virus purification. To harvest cells, EDTA (Thermofisher) was added to a final concentration of 6.25 mM to promote cell detachment from the tissue culture plastic. rAAV virus was purified via iodixanol gradient ultracentrifugation or the AAVpro® Purification Kit (Takara) according to the manufacturers instructions.

For iodixanol gradient purification cell pellets were collected by centrifugation (300 g, 5 min), resuspended in 5 ml cell lysis buffer (150 mM NaCl, 5 mM Tris-HCl pH8.5) and subjected to three rounds of freeze thaw using a dry-ice/ethanol bath and a 37° C. water bath. Following the final thaw, the lysate was treated with Benzonase (200 U/ml) (Sigma) for 1 hr at 37° C. Remaining debris was removed by centrifugation (1000 g, 29 min, 4° C.) before loading onto the iodixanol gradient. The iodixanol gradient (9 ml 15% iodixanol, 6 ml 25% iodixanol, 5 ml 40% iodixanol and 5 ml, 60% iodixanol) was prepared by diluting 60% iodixanol (OptiPrep), (STEMCELL Technologies). The 25% and 40% iodixanol phases were prepared by diluting 60% iodixanol in PBS-MK buffer (1×PBS, 2.76 mM MgCl2, 2 mM KCl). The 15% iodixanol phase was prepared by diluting 60% iodixanol in PBS-MK buffer (1×PBS 1 M NaCl, 2.76 mM MgCl₂, 2 mM KCl). Phenol red was added to the 25% and 60% phases into order to distinguish these layers. Gradient phases are added step wise into OptiSeal Polypropylene Centrifuge tubes (Beckman Coulter). The cell lysate is loaded on top of the 15% phase and tubes centrifuged in a 70Ti ultracentrifuge rotor at 67,000 rpm for 90 min at 18° C. rAAV is extracted from the 40% phase following centrifugation. rAAV virus preps were concentrated further using centrifugal filter units (MWCO 100 kDa) (Millipore) and the buffer was exchanged for PBS 0.001% (v/v) Pluronic Acid (Sigma). Virus preps were stored at −80° C.

Viral titres were confirmed by qPCR using the QuantiTect Probe PCR kit (Qiagen). Primer and probe sequences are designed against the ITR (Aurnhamer et al., 2012. Human Gene Ther Methods). Primer sequences ITR_F GGAACCCCTAGTGATGGAGTT, SEQ ID NO: 54, ITR_R CGGCCTCAGTGAGCGA SEQ ID NO: 55, Probe 56-FAM/CACTCCCTC/ZEN/TCTGCGCGCTCG/3IABkFQ. Viral quantification was determined against a standard curve prepared from pAAVS derived plasmid DNA of known concentration and copy number. The PCR program consisted of one cycle of denaturation at ° C. for 15 min followed by 40 cycles of denaturation at 95° C. for 15 s and primer annealing/extension at 60° C. for 60 s. qPCR reactions were performed on the QuantStudio 7 and analysed with the QuantStudio™ Real Time PCR software.

Gene Editing—Knock-In of A2M4 SPEAR into Exon 33 at the PTPRC Locus iPSC lines CHiPSC31 and ADAPi001-J were passaged enzymatically using TrypLE Select (ThermoFisher) as a single cell suspension. Following dissociation, iPSCs were counted (NucleoCounter NC-250). CHiPSC31 (200-300×10³) were seeded in a well of a coated (Coati) 6-well plate containing complete DEF-CS 500 supplemented with GF3. Media was exchanged for complete DEF-CS 500 the following day. ADAPi001-J (2-3×10⁵) were seeded into a well of a Synthemax (Corning) coated 6 well plate containing mTeSR Plus (STEMCELL Technologies) supplemented with 1× CloneR (STEMCELL Technologies), Media was exchanged for mTeSR Plus the following day. iPSC s (CHiPSC31 or ADAPi001-J) were cultured for 48 hours prior to AAV transduction. CHiPSC31 or ADAPi001-J were transduced with AAV3 serotyped virus (500-10000 Vg/cell). Antibiotic selection (Geneticin, 30-100 μg/ml) was commenced 48 hrs post transduction and was continued throughout the remaining culture. 3-6 days after viral transduction, iPSC s were seeded into coated 96 well plates for screening and clonal isolation. Clones were expanded in 96 well plates for 10-16 days before PCR screening. In some instances, positive clones were subjected to additional clonal isolation by limiting dilution. All clones were screed by PCR in order to confirm integration. All PCR products were sequence verified.

### Clone Isolation and Screening

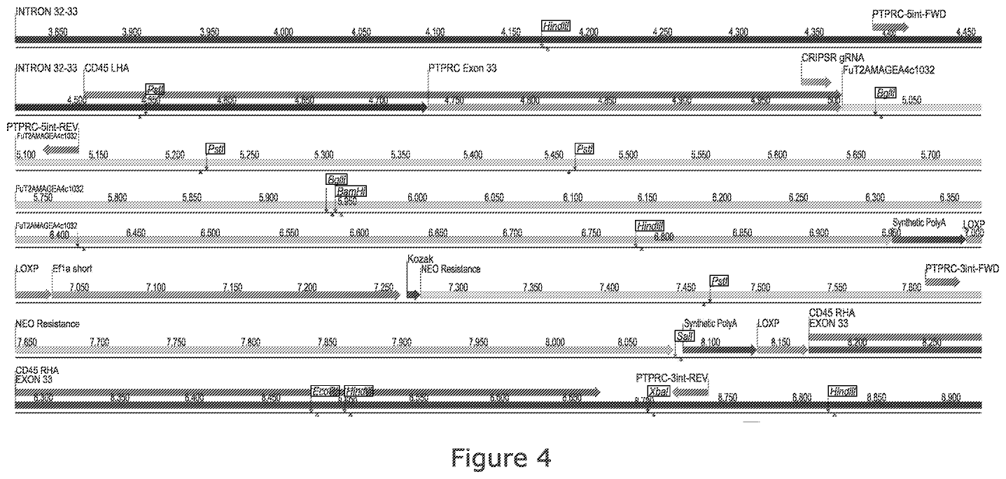

iPSC clones were isolated following selection of the edited iPSC cell pools with G418. Edited ChiPSC31 (3000 cells) were seeded into 10 cm dishes with complete DEF-CS 500 supplemented with GF3 and G418 (50 μg/ml). Media was exchanged every 48 hrs for complete DEF-CS 500 supplemented with G418 (50 μg/ml). Clones were manually picked following 11 days of expansion. A sample was taken for genomic DNA extraction using QuickExtract™ (Lucigen). Clones were screened for transgene integration by PCR (FIG. 4).

Edited clones from the ADAPi001_J line were isolated by limiting dilution in 96 well plates coated with Synthemax™. Cells were seeded at an average of 100 cells/well. 9600 iPSC were seeded in well A1 before 2× serial dilution in column 1 (A1-H1). Cells were then diluted 2× across all rows of the plate. Edited ADAPiJ001_J were seed into mTeSR Plus supplemented with 1× CloneR. Media was exchanged every 2 days for complete mTeSR Plus. Cells were cultured for 12-14 days before screening wells for the presence of single colonies. Clones were expanded and genomic DNA isolated using QuickExtract™ (Lucigen) for screening by PCR in order to confirm targeted transgene insertion.

PCR reactions were performed across homology arm boundaries. The 5′ LHA boundary was amplified with the primers PTPRC-5int-FWD CAGTGATTCCTGCCCTGATTCTTA (SEQ ID No: 50) and PTPRC-5int-REV TACAGCCACAGGATCACGAGAAAG (SEQ ID No: 51). The primer PTPRC-5int-FWD corresponds to nucleotides 198755562 to 198755585 (+) on chromosome 1 (Genome build GRCh38 Ensembl release 99—January 2020) and lies 93 nucleotides upstream of the LHA sequence. The PTPRC-5int-REV primer is in the A2M4 transgene sequence. The 3′ RHA boundary was amplified with the primers PTPRC-3int-FWD CCTGCCGAGAAAGTATCCATCAT (SEQ ID No: 52) and PTPRC-3int-REV TAGCATACACACACATACCACCTT (SEQ ID No: 53). The primer PTPRC-3int-FWD1 is in the NEO cassette and PTPRC-3int-REV1 corresponds to nucleotides 198756729 to 198756752 (−) on chromosome 1 and lies 47 nucleotides downstream from the RHA. PCR reactions were performed with Q5® Hot Start High-Fidelity 2× Master Mix (NEB). PCR program denaturation 98° C., 30 s, 1 cycle, 40 cycles denaturation 98° C., 5s, annealing 66° C., 10s, Extension 72° C., 25 s, 1 cycle extension 72° C., 5 min. PCR products were gel purified and sanger sequenced to confirm integration. Positive clones were expanded, and small cell banks produced. Maintenance of pluripotency marker expression was confirmed by flow cytometry. Three clones were progressed for differentiation. ChiPSC31 PTPRC_A2M4 clone 2, ADAPiJ001_J PTPRC_A2M4 clone 3D10 and ADAPiJ001_J PTPRC_A2M4 clone 3G5. ChiPSC31 PTPRC_A2M4 clone 2 and ADAPiJ001_J PTPRC_A2M4 clone 3G5 had targeted knock-in of ADP A2M4 at one allele whereas ADAPiJ001_J PTPRC_A2M4 clone 3D10 had ADP A2M4 knock-in at both alleles.

### T Cell Differentiation

HiPSC maintenance medium was removed, the cells washed twice with DMEM/F12 (Invitrogen). 2 mL of StemPro34 PLUS (StemPro34 from Invitrogen; StemPro34 basal media, with supplement added and Penicillin Streptomycin (1% v/v: Invitrogen) and Glutamine (2 mM: Invitrogen), Ascorbic Acid (50 mg/ml: Sigma Aldrich) and monothioglycerol (100 mM: Sigma Aldrich), further supplemented with 50 ng/mL of Activin A (Miltenyi Biotec) was added and incubated for 4 hours. Volumes are dependent of culture flask size, typically at least 2 mls/9 cm², and 20 mls/150 cm².

After 4 hours, the medium was removed, and the cells washed twice with DMEM/F12 to remove residual high concentration Activin A. The medium was replaced with 2 mL of StemPro34 PLUS supplemented with 5 ng/mL of Activin A, 10 ng/ml of BMP4 (Miltenyi Biotec) and 5 ng/ml of bFGF (Miltenyi Biotec) and incubated for 44 hours (Stage 1 media). The medium was then replaced with fresh Stage 1 media and supplemented with 10 μM CHIR-99021 (Selleckchem) and further cultured for 48 hours.

On Day 4, the medium was removed, and the cells washed twice with DMEM/F12 to remove residual stage 1 cytokines. The medium was then replaced with StemPro34 PLUS supplemented with 100 ng/mL of SCF (Miltenyi Biotec) and 15 ng/ml of VEGF (Miltenyi Biotec) and incubated for 48 hours (Stage 2 media). The medium was then replenished with fresh Stage 2 media and the cells cultured for a further 48 hours.

The medium was then replaced by the Stage 3 medium which requires the minimum of SCF and VEGF and the cells cultured for between 16-18 days, with demi depletion feeding every 48h. Typically this involved harvest of media and collection of cells in suspension by centrifugation (300 g, 10 min), and returning suspension cells to culture with fresh media (i.e 20 ml for a T150 flask).

On approximately day 16 CD34+ cells were isolated from resulting monolayers for onward culture. CD34+ cells were harvested by sequential incubation with Accutase (STEMCELL Technologies) for 30 mins at 37° C. and then Collagense II (Invitrogen: 2 mg/ml) for 30 mins at 37° C. Cell suspensions were collected and washed (×2 centrifugation at 300 g for 12 min in DMEM/F12), prior to CD34+ cell isolation via Magnetic activated beads (MACS) isolation (Miltenyi: according to manufacturer's instructions).

For continued lymphoid proliferation and differentiation, STEMCELL Technologies proprietary 2 stage (Lymphoid Proliferation/T cell Maturation) media was employed.

During culture in STEMCELL Technologies Lymphoid Proliferation media T-cell progenitors non-edited control lines were lentivirally transduced with the ADP A2M4 TCR in the presence of poloxymer F108 (Sigma).

### T Cell Phenotyping

iPSC derived T Cells were phenotyped using flow cytometry. The cells were stained with CD3 (clone SK7); A2M4 dextramer and live/dead stain EF506 to show expression of ADP A2M4 TCR in iPSC derived T Cells.

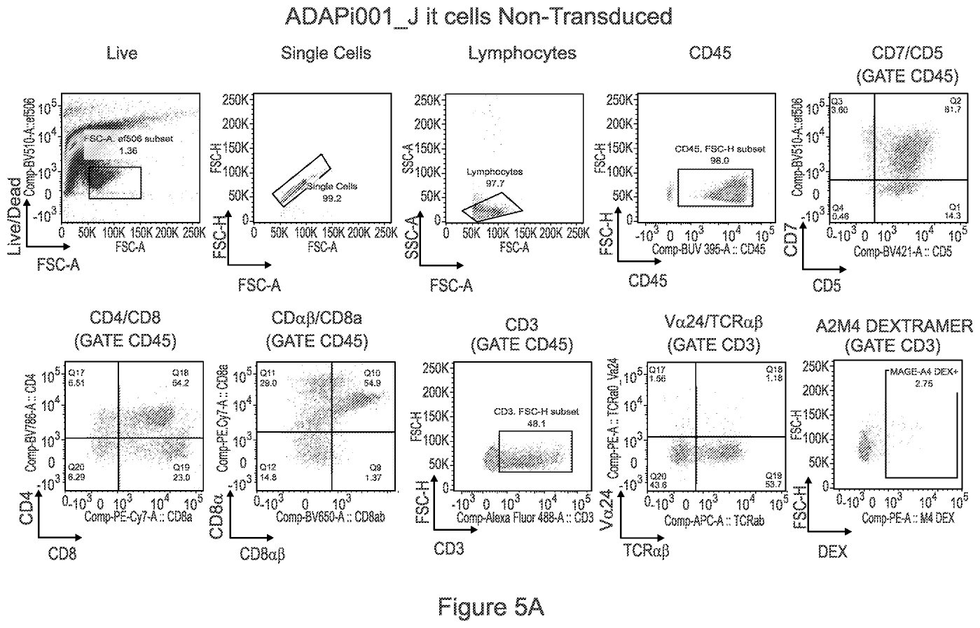

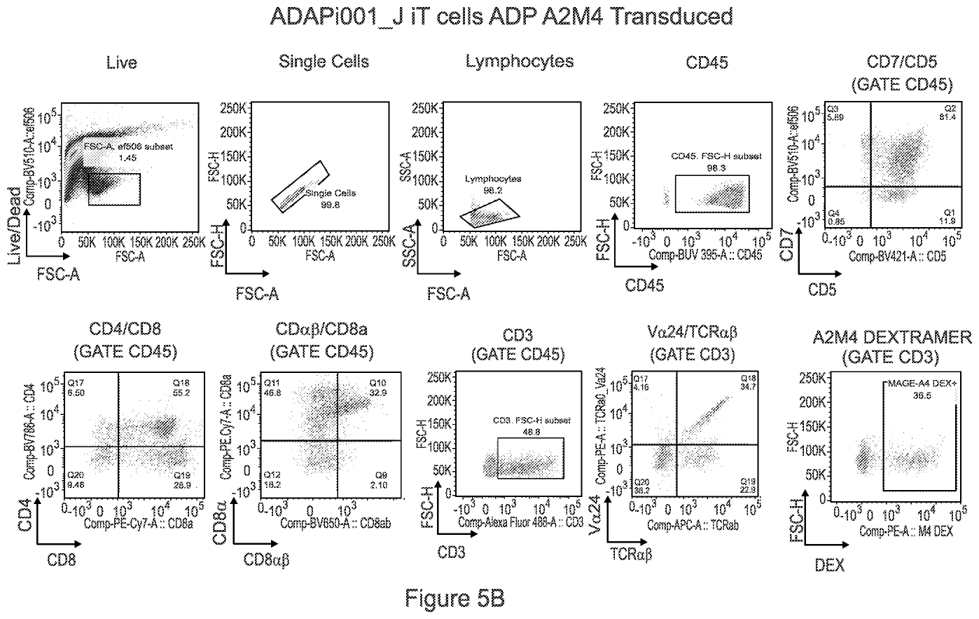

FIG. 5 shows phenotyping data for transduced clones. Expression of ADP A2M4 in iT cells can be induced via lentiviral transduction of CD7+CD5+ progenitors as described above. The iPSC line ADAPi001_J was differentiated and transduced with lentivirus express containing the ADP A2M4 T cell at Stage 4 (CD7+CD5+ progenitor) and the transduced cells were progressed through the differentiation protocol. Cells were phenotyped by FACS and ADP A2M4 expression measured by staining with anti-TCR antibody anti-Vα24 and MAGE-A4 GVYDGREHTV/HLA-A*0201 dextramer.

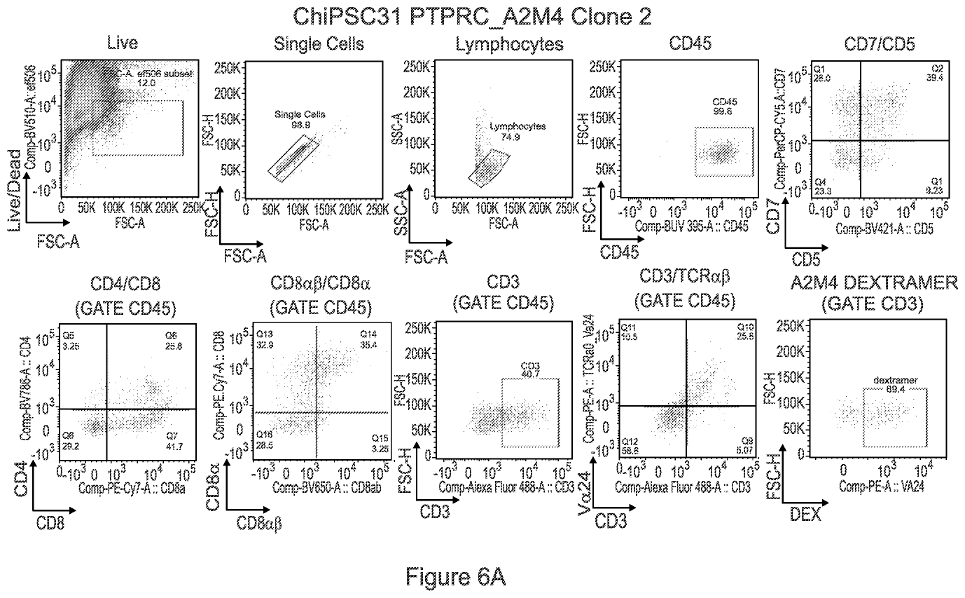

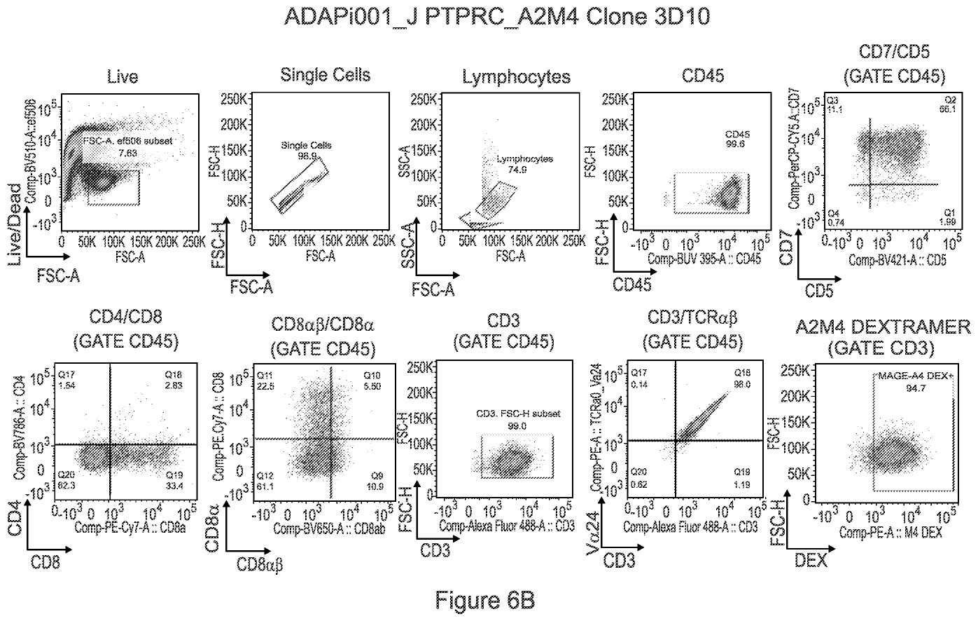

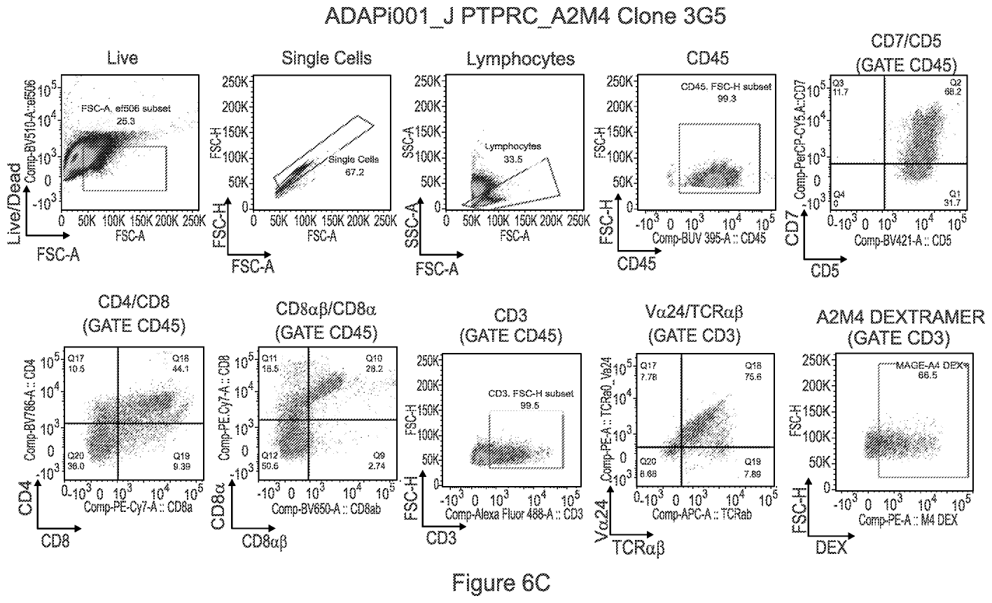

FIG. 6 shows phenotyping data for iT cells differentiated from edited iPSC clones (Stage Panel (A) shows iT cells differentiated from an alternative cell line ChiPSC31 PTPRC_A2M4 clone 2 (single targeted allele), panel (B) iT cells differentiated from ADAPi001_J PTPRC_A2M4 clone 3D10 (both alleles targeted) and panel (C) iT cells differentiated from ADAPi001_J PTPRC_A2M4 clone 3G5 (single targeted allele). Differentiation is unaffected by editing. iT-cells were phenotyped by flow cytometry and expression of CD45, CD7, CD5, CD4, CD8, CD56 and CD3 were detected. Edited clones express the ADP A2M4 TCR. Cells were phenotyped by FACS and A2M4 expression measured by staining with anti-TCR antibody anti-Vα24 and MAGE-A4 GVYDGREHTV/HLA-A*0201 dextramer. Knock-in at a single allele is sufficient to promote ADP A2M4 TCR expression, where both alleles are targeted the expression is improved.

### Flow Cytometry Staining

The following antibodies were used for flow cytometry: CD8αβ-BV650 (clone 2ST8.5H7), TCRγδ-APC (done B1), TCR Vα24-PE (Clone IP26), CD3-APC-R700 (clone UCHT1), CD4-BV605 (clone RPA-T4), CD8α-PE-CY7 (clone RPA-T8), CD69-BUV395 (clone FN50), CD25-BV421 (Clone BC96), HLA-DR-AF488 (clone L243), and EF506-BV510. Samples were acquired on the BD Fortessa.

### KILR Cytotoxicity Assay

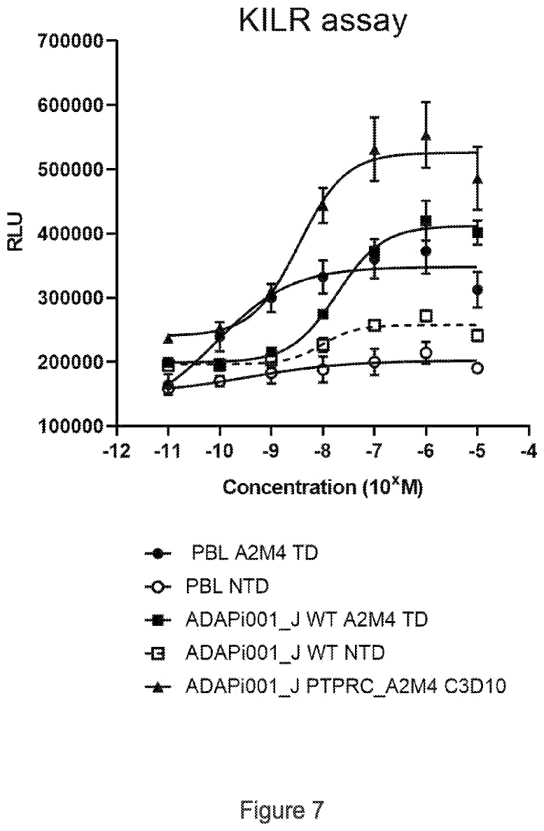

10,000 KILR T2s (generated by transduction with KILR retroparticles (DiscoverX)) were added per well of a 384 well white LUMITRAC 600 microplate (Greiner). T cells derived from iPSC lines at the end of stage 6 were added at 20,000 cells per well. MAGE-A4 peptide (GVYDGREHTV) was added to cells in a titration from 10⁻⁵ M to 10⁻¹¹ M. Cells and peptide were co-cultured for 24 hours at 37° C. under normoxia before addition of KILR detection solution (KILR Detection Kit (DiscoverX)) for 1 hr at room temperature. Luminescence from samples was detected using the FLUOstar Omega Microplate Reader. The cytolytic activity of iT cells was compared to non-transduced and ADP A2M4 lentivirus transduced PBL controls from healthy donors. FIG. 7 shows data for iT cells differentiated from ADAPi001_J (ADAP001_J WT NTD (non-transduced)), ADAPi001_J derived, iT cells transduced with ADP A2M4 lentivirus (ADAPi001_J WT A2M4 TD) and iT cells differentiated from edited ADAPi001_J PTPRC_A2M4 clone 3G10. Increased signal indicates increased cell death and the data demonstrate potent cytolytic effector function of the knock-in edited ADAPi001_J PTPRC_A2M4 clone 3G10.

Edited ADAPi001-J ADP A2M4 3D10 iT-cells, ADP A2M4 transduced and non-transduced ADAPi001-J (WT) derived iT-cells were also co-cultured with A375 (MAGE-A4+HLA-A*02+) or COLO205 (MAGE-A4− HLA-A*02+) in the presence or absence of MAGE-A4 peptide (GVYDGREHTV) for 24 hrs. Cytokines. (IFNγ and IL-2) and Granzyme B secretion was measured by ELISA. The edited ADP A2M4 iT-cells were seen to release high levels of each cytokine in an antigen dependent manner.

### Antigen-Specific Activation Assay

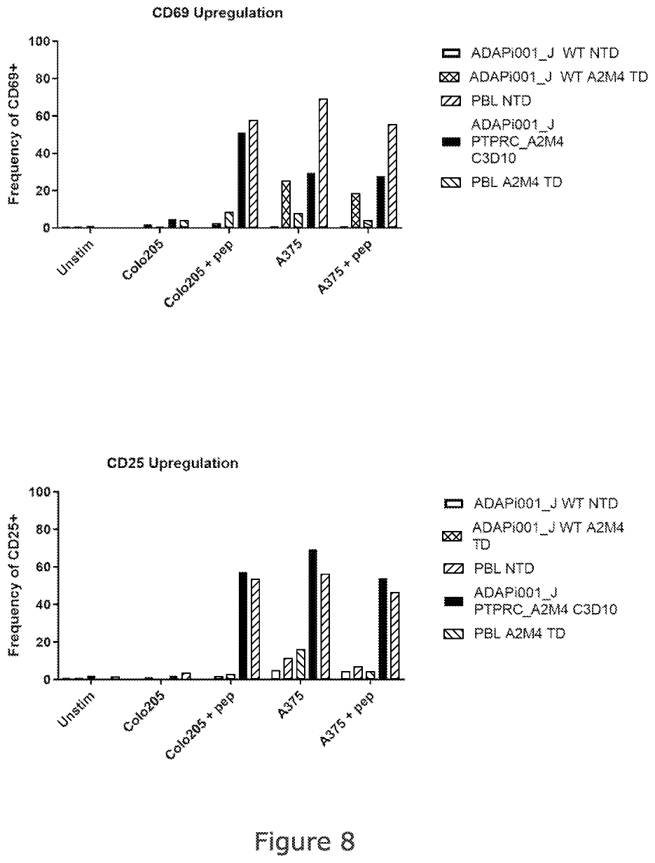

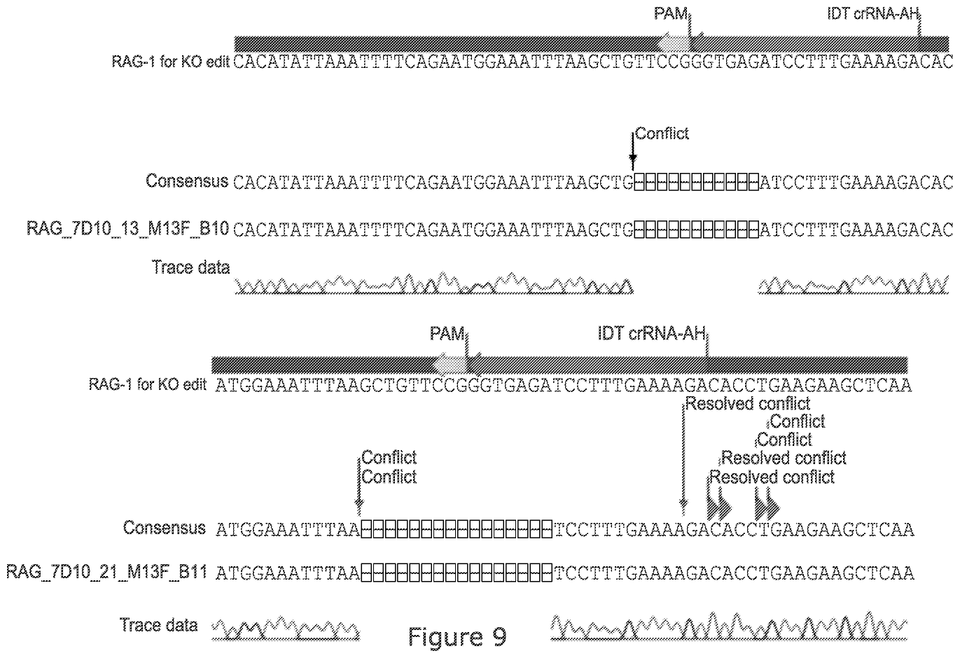

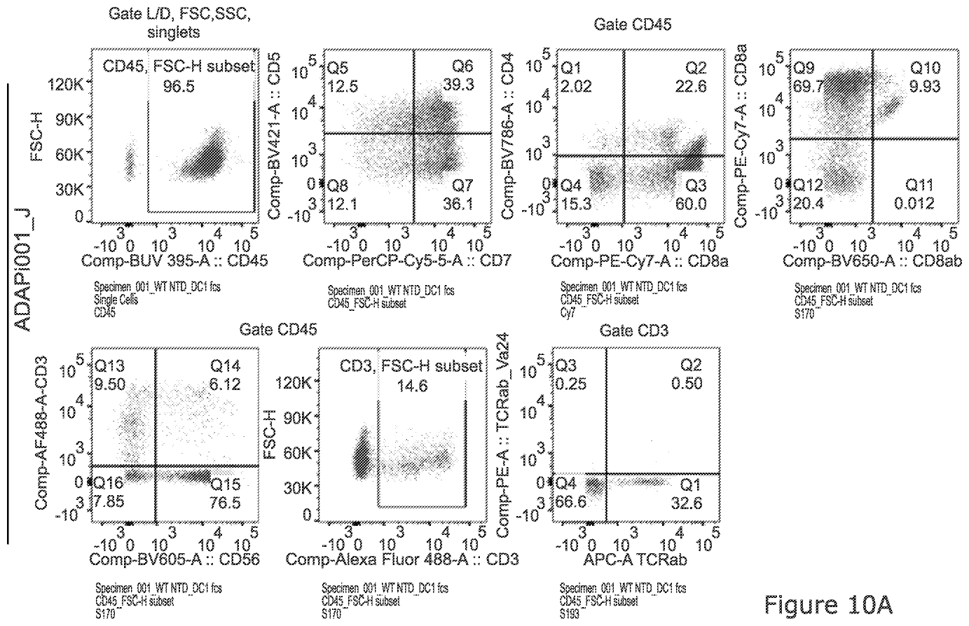

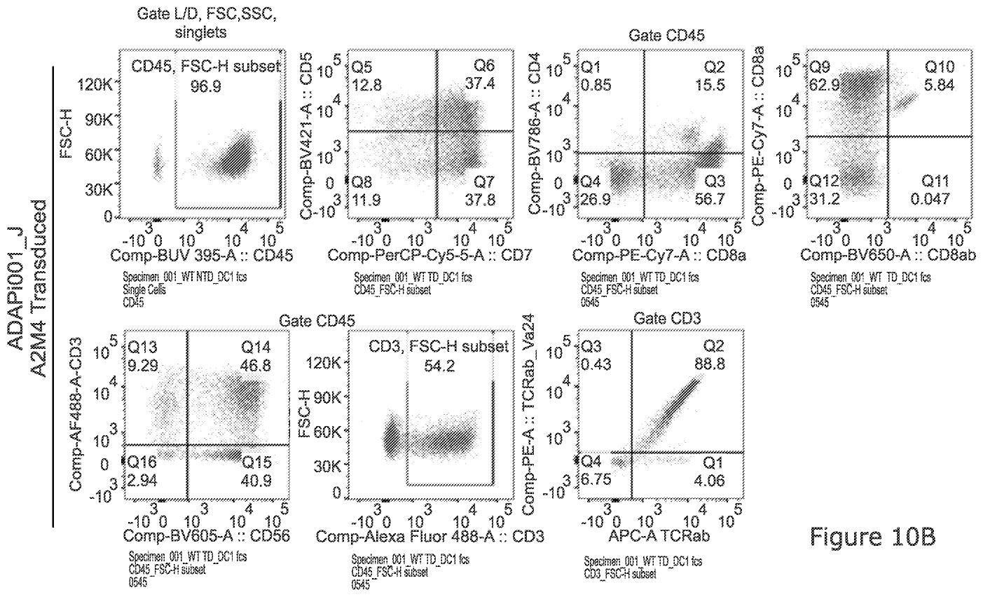

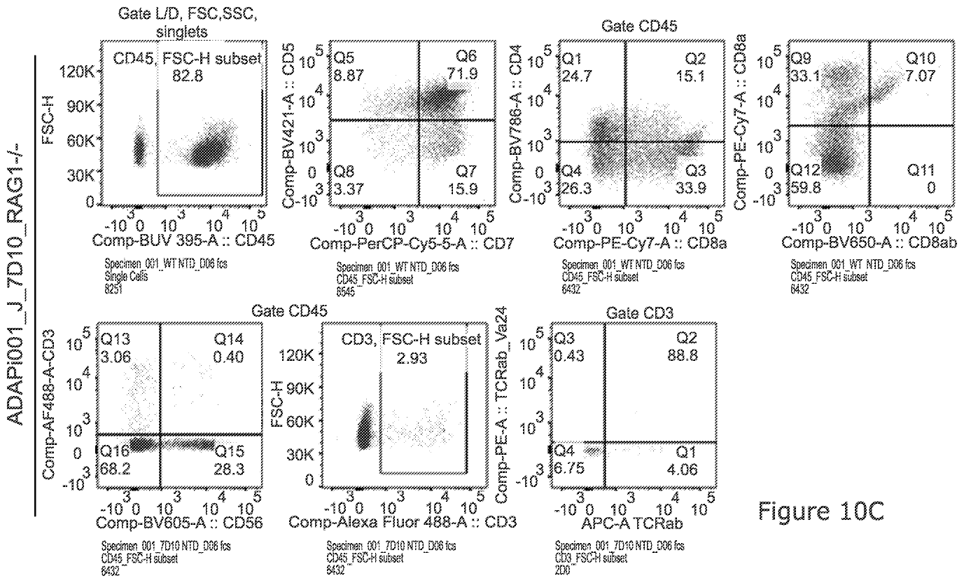

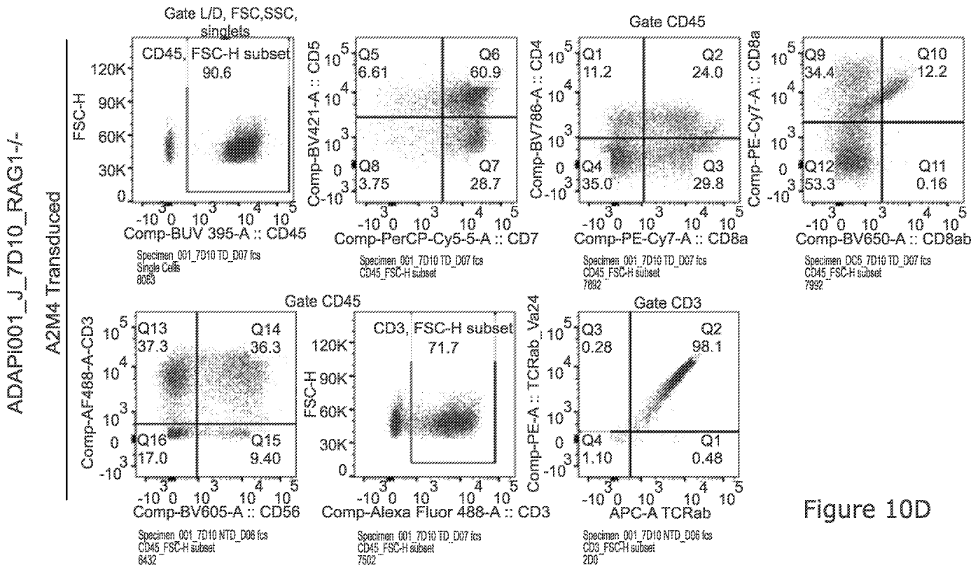

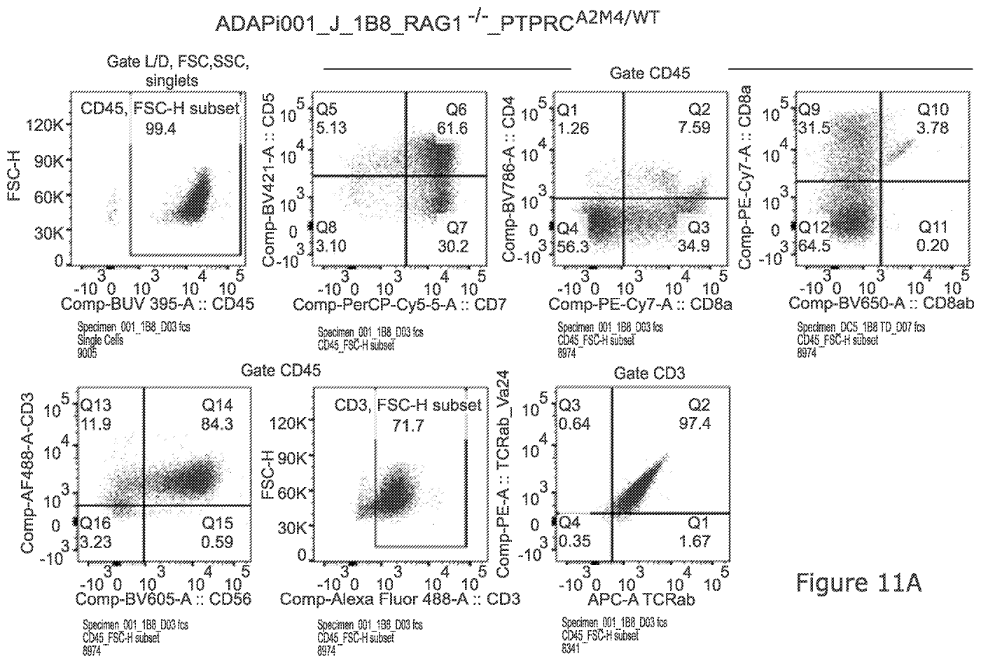

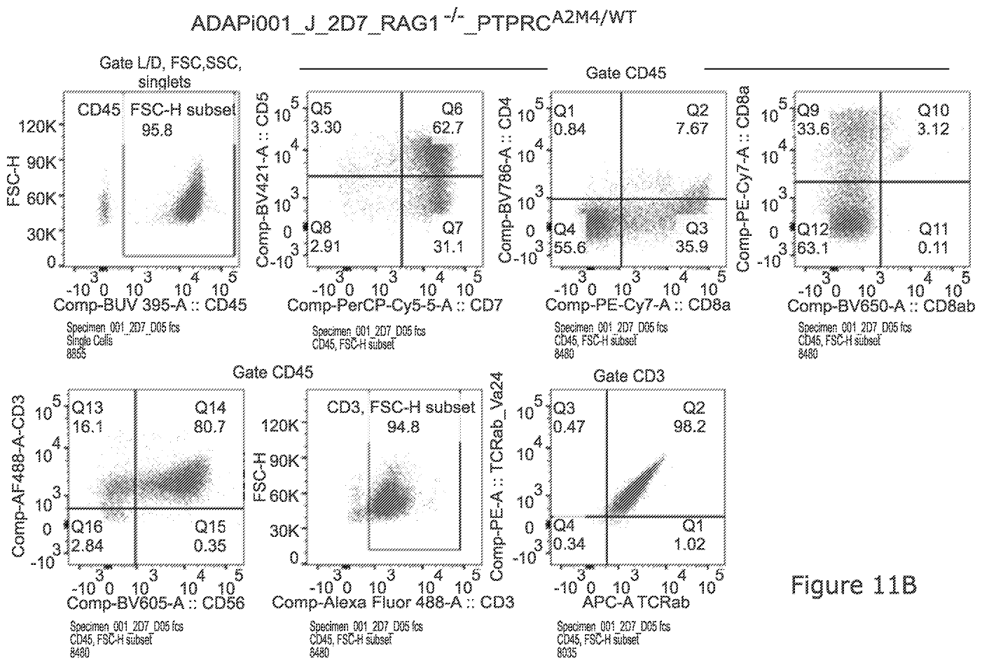

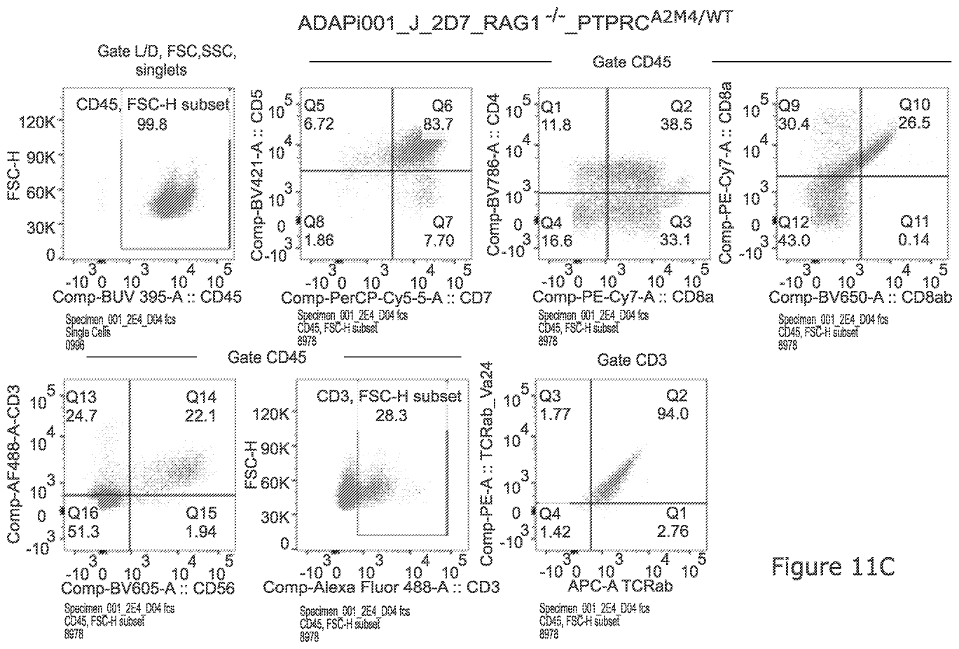

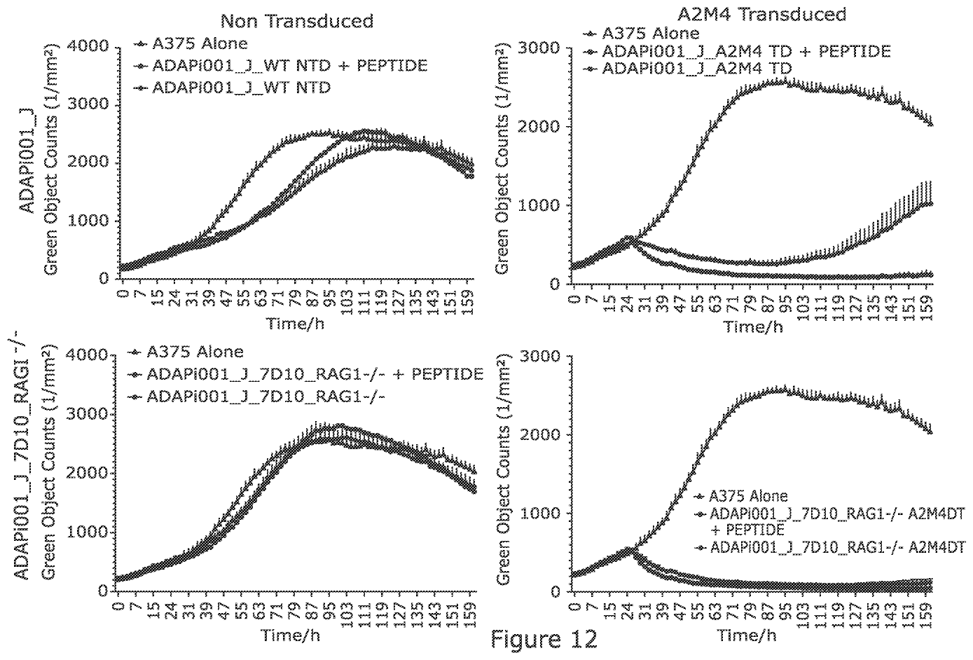

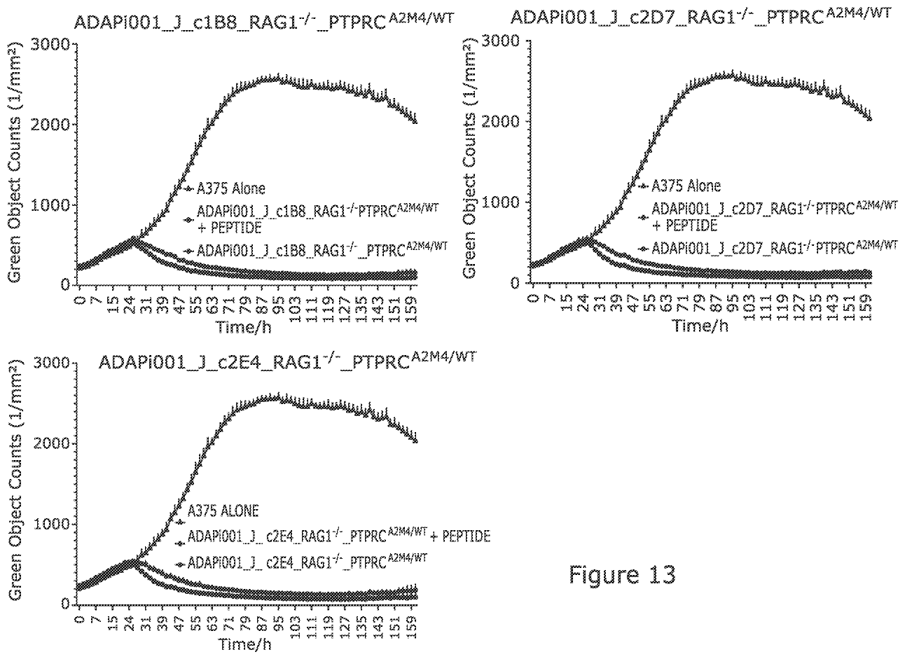

A375 (HLA-A*02 positive and MAGE-A4 positive) and Colo205 (HLA-A*02 positive and MAGE-A4 antigen negative) tumour cell lines were used as target cells. 200,000 targets were co-cultured in a 96 well round bottom plate, with either 1×10\hat{ }6 iPSC derived T cells, or PBL controls (rested over 2 hours). Target cell lines and T cells were then cultured with or without 0.5 μg of ADP-A2A4 peptide (GVYDGREHTV). Additionally, iPSC derived T cells were cultured without peptide and target cells. All cells were then incubated at 37° C. for 24 hours prior to cell staining. Following co-culture, up-regulation in expression of the T cell activation markers CD69 and CD25 was determined by flow cytometry. iT cells were compared to non-transduced and ADP A2M4 lentivirus transduced PBL controls (FIG. 8). The edited ADP A2M4 iT-cells were seen to up-regulate the activation markers CD69 and CD25 when incubated with antigen positive tumour cells lines.

### RAG1 Null Cell Knock-In

iPSC Cell Culture

Sequential RAG1 editing and knock-in of the A2MR TCR into the PTPRC locus were performed the iPSC line ADAPi001_J. The ADAPi001_J iPSC line was cultured on the Synthemax™ II-SC Substrate (Corning) with mTeSR™ Plus culture media (STEMCELL Technologies). Both iPSC lines were adapted for enzymatic dissociation with TrypLE™ Select (ThermoFisher) and passaged every 4-5 days. iPSC cultures were maintained in a humidified 37° C., 5% 02, 5% CO₂ incubator. The expression of pluripotency markers (POU5F1, NANOG, TRA-160 and SOX2) and absence of the differentiation marker SSEA-1 was routinely monitored by FACS analysis. Editing experiments were performed sequentially. Clone 7D10 with RAG1 inactivation was derived from wild type ADAPi001_J. Clones 167, 2D7 and 2E7 (RAG1^{-/-} PTPRC^{A2M4/WT}) were generated via knock-in of the A2M4 TCR into PTPRC Exon 33 into the RAG1^{-/-} clone 7D10.

### Guide RNA Sequences

RAG1 Exon 2 was targeted with the guide RNA TCTTTTCAAAGGATCTCACC. This sequence corresponds to nucleotides 36,573,405-36,573,425, chromosome 11 (Human GRCh38 Ensembl release 103—February 2021). Exon 33 PTPRC was targeted with the guide RNA GCAAGTCCAGCTTTAAATCA (nucleotides 198756151-198756171, Chromosome 1. Human GRCh38, Human GRCh38 Ensembl release 103—February 2021). Guide RNA sequences were synthesised by IDT.

### Preparation of Ribonucleoprotein (RNP) Complexes

crRNA and tracrRNA were annealed by initial denaturation 95° C. for 5 min before cooling to room temperature. Equimolar quantities of annealed crRNA/tracrRNA duplexes and Cas9 protein were incubated at room temperature for 15 min to generate 10 μM Ribonucleoprotein (RNP) complexes.

### Inactivation of RAG1

RNP complexes were introduced into iPSC cells via nucleofection with the 4D-Nucleofector™ using the 16-well Nucleocuvette™ strips (Lonza). 100×10³ ADAPi-001_J (Wildtype) were resuspended in buffer P3 (Lonza) (5×10⁶/ml). 3 μl of RNP complex (10 μM) was added to 20 μl cell suspension. Nucelofection was performed with program CA-137. Following nucleofection cells were immediately seeded into mTeSR™ Plus culture media (STEMCELL Technologies) supplemented with 1× CloneR™ (STEMCELL Technologies). Edited cell pools were expanded for two additional passages before seeding for clonal isolation. Cells were seeded at an average of 100 cells/well. 9600 iPSC were seeded in well A1 before 2× serial dilution in column 1 (A1-H1). Cells were then diluted 2× across all rows of the plate. Edited ADAPiJ001_J were seed into mTeSR Plus supplemented with 1× CloneR™. Media was exchanged every 2 days for complete mTeSR Plus. Cells were cultured for 12-14 days before screening wells for the presence of single colonies. Clones were expanded and genomic DNA isolated using QuickExtract® (Lucigen) for screening by PCR in order to sequence the INDELS in RAG1 (Exon2). PCR reactions (NEB Q5 HotStart) were performed using FWD primer CTTGGGACTCAGTTCTGCCC (Chromosome 11 nucleotides 36573327-36573346 (+) Human GRCh38 Ensembl release 103—February 2021) and REV primer GAACTCAGTGGGGTGGATCG. Chromosome 11 nucleotides 36573809-36573829 (−) Human GRCh38 Ensembl release 103—February 2021). PCR reactions were performed with 05® Hot Start High-Fidelity 2× Master Mix (NEB). PCR program denaturation 98° C., 30 s, 1 cycle, 40 cycles denaturation 98° C., 5s, annealing 69° C., 10s, Extension 72° C., 30 s, 1 cycle extension 72° C., 2 min. PCR products from each clone were cloned into TOPO Blunt (Thermofisher) and plasmid clones were sanger sequenced to characterise the INDELS present in each edited iPSC clone. This analysis revealed that clone ADAPi001_J_c7D10_RAG1^{-/-} had a 11 bp deletion in 1 allele and a 16 bp deletion in the second allele. Both mutations are predicted to introduce inactivating frameshift mutations into RAG1 (FIG. 1).

Knock-In of A2M4 TCR into PTPRC (Exon 33)—ADAPi001_J_c7D10_RAG1^{-/-}

Knock-in of A2M4 into the PTPRC locus (Exon 33) was performed using CRISPR Cas9 and rAAV supplied repair templates. The editing experiment was performed with the gRNA sequence gcaagtccagctttaaatca (nucleotides 198756152 to 198756171 on Chromosome 1. Human GRCh38, Ensembl release 103—February 2021). crRNA was synthesised by Integrated DNA Technologies.

1×10⁵ iPSC were seeded in a well of a twelve well plate 24 hrs before transfection. Cells were seeded in complete mTeSR-Plus (STEMCELL Technologies) supplemented with 1× ClonerR™ (STEMCELL Technologies). The transfection mix was prepared according to manufacturer's instructions. 500 ng of annealed crRNA/tracrRNA duplexes and 2.5 μg Cas9 protein were added to 50 μl Optimem (Thermofisher) and 5 μl Cas9 and incubated at room temperature for 15 min to generate Ribonucleoprotein (RNP) complexes. 3 μl Lipofectamine CRISPR-MAX (Thermofisher) in 50 μl Optimem was added following RNP complex formation and then the complete transfection mix added to cells. 4 hrs post transfection iPSC were transduced with rAAV (Serotype3) encoding PTPRC_FuT2A_A2M4_LOXPNEO (1×3 Vg/cell). Media was exchanged for complete mTeSR-Plus (STEMCELL Technologies) supplemented with 100 μg/ml G418 (Thermofisher) 24 hrs post rAAV transduction. Edited cell pools were expanded for 1 passage before clonal isolation with limiting dilution in 96 well plates coated with Synthemax™. 1.5×10⁴ iPSC were seeded in well A1 before 2× serial dilution in column 1 (A1-H1). Cells were then diluted 2× across all rows of the plate. Edited iPSC were seeded into mTeSR Plus supplemented with 1× CloneR™. Media was exchanged every 2 days for complete mTeSR Plus supplemented with 100 μg/ml G418 (Thermofisher). Cells were cultured for 12-14 days before screening wells for the presence of single colonies. Clones were expanded and genomic DNA isolated using QuickExtract™ (Lucigen) for screening by PCR in order to confirm targeted transgene insertion.

PCR reactions were performed across homology arm boundaries. The 5′ LHA boundary was amplified with the primers PTPRC-5int-FWD CAGTGATTCCTGCCCTGATTCTTA and PTPRC-5int-REV TACAGCCACAGGATCACGAGAAAG. The primer PTPRC-5int-FWD corresponds to nucleotides 198755562 to 198755585 (+) on chromosome 1 (Human GRCh38, Ensembl release 103—February 2021) and lies 93 nucleotides upstream of the LHA sequence. The PTPRC-5int-REV primer is in the A2M4 transgene sequence. The 3′ RHA boundary was amplified with the primers PTPRC-3int-FWD CCTGCCGAGAAAGTATCCATCAT and PTPRC-3int-REV TAGCATACACACACATACCACCTT. The primer PTPRC-3int-FWD1 is in the NEO cassette and PTPRC-3int-REV1 corresponds to nucleotides 198756729 to 198756752 (−) on chromosome 1 and lies 47 nucleotides downstream from the RHA. (Human GRCh38, Ensembl release 103—February 2021). PCR reactions were performed with Q5® Hot Start High-Fidelity 2× Master Mix (NEB). PCR program denaturation 98° C., 30 s, 1 cycle, 40 cycles denaturation 98° C., 5s, annealing 66° C., 10s, Extension 72° C., 25 s, 1 cycle extension 72° C., 5 min. PCR products were gel purified and sanger sequenced to confirm integration. Positive clones were expanded, and small cell banks produced. Maintenance of pluripotency marker expression was confirmed by flow cytometry

### IncuCyte® Cytotoxicity Assay

GFP^{+ve} A375 (MAGE-A4^{+ve}, HLA-A2*02^{+ve}) targets cells were seeded in 384 well plates (Greiner Bio-One) at a density of 1500/well in 200 of complete RPMI 1640 (RPMI 1640, 10% (v/v) FCS, 100 U/ml penicillin 100 μg/ml streptomycin, 2 mM L-Glutamine). Plates were added to the Incucyte Zoom, and incubated at 37° C., with 5% CO₂ in a humidified incubator.

24 hours later, targets were pulsed with MAGE-A4 peptide (GVYDGREHTV) at a final concentration of 10 μM. 6000 iT cells were added in triplicate in 10 μl media. Images were taken every 2 hours, for six days in the Incucyte. Data was then analysed using ZOOM2018A software using Top-Hat process definitions.

### CONCLUSION

We have presented a novel editing strategy that has enabled the differentiation of ADP A2M4 TCR expressing iT-cells. Edited ADP A2M4 TCR iT-cell activation and cytolytic effector function has been shown to be antigen dependent. The phenotype of edited ADP A2M4 iT-cells including CD56 and CD8αα expression, potent cytokine and cytotoxic activity suggests that edited ADP A2M4 iT-cells possess an innate and adaptive like phenotype.

Although dextramer staining suggests that edited ADP A2M4 iT-cells only express the ADP-A2M4 TCR the technology is applicable to a full TCR repertoire. The edited A2M4 TCR iT-cells exhibit potent cytolytic and effector function, which is comparable or increased compared to A2M4 TCR transduced PBL from healthy donors. This suggests that, like autologous A2M4 TCR T-cells, ADP-A2M4 iT-cells will be effective in cell therapy and bring clinical benefit to patients.

The generation ADP-A2M4 iT-cells is an important milestone in producing an iPSC derived allogeneic platform. The ability to promote TCR expression in iT-cells via genetic knock-in at a single, defined locus offers an opportunity to produce multiple clonal iPSC banks encoding specific TCRs against a range of tumour antigens. In the future, this “off-the-shelf” platform could deliver a range of defined and consistent T-cell therapies to patients specific to their tumour antigen expression profile in a timely manner.

Sequences

SEQ ID NO: 1, MAGE A4

| | | | | |
|---|---|---|---|---|
| MSSEQKSQHC | KPEEGVEAQE | EALGLVGAQA | PTTEEQEAAV | SSSSPLVPGT |
| LEEVPAAESA | GPPQSPQGAS | ALPTTISFTC | WRQPNEGSSS | QEEEGPSTSP |
| DAESLFREAL | SNKVDELAHF | LLRKYRAKEL | VTKAEMLERV | IKNYKRCFPV |
| IFGKASESLK | MIFGIDVKEV | DPASNTYTLV | TCLGLSYDGL | LGNNQIFPKT |
| GLLIIVLGTI | AMEGDSASEE | EIWEELGVMG | VYDGREHTVY | GEPRELLTQD |
| WVQENYLEYR | QVPGSNPARY | EFLWGPRALA | ETSYVKVLEH | VVRVNARVRI |
| AYPSLREAAL | LEEEEGV | | | |

```
SEQ ID NO: 2, MAGE A4 peptide
GVYDGREHTV
SEQ ID NO: 3; (CD8α) CDRs bold underlined, signal sequence italic
underlined
MALPVTALLLPLALLLHAARPSQFRVSPLDTWNLGETVELKCQVLLSNPTSGCSWLFQPRGAAASPT
FLLYLSQNKPKAAGLDTQRFSCKRLGDTFVLTLSDFRRENECYYFCSALSNSIMYFSHFVPVFLPAK
PTTTPAPRPPTPAPTIASQPLSLRPEACRPAAGAVHTRGLDFACDIYIWAPLAGTCGVLLLSLVITL
YCNHRNRRRVCKCPRPVVKSGDKPSLSARYV
SEQ ID NO: 4; (CD8α)
ATGGCCTTACCAGTGACCGCCTTGCTCCTGCCGCTGGCCTTGCTGCTCCACGCCGCCAGGCCGAGCCA
GTTCCGGGTGTCGCCGCTGGATCGGACCTGGAACCTGGGCGAGACAGTGGAGCTGAAGTGCCAGGTGC
TGCTGTCCAACCCGACGTCGGGCTGCTCGTGGCTCTTCCAGCCGCGCGGCGCCGCCGCCAGTCCCACC
TTCCTCCTATACCTCTCCCAAAACAAGCCCAAGGCGGCCGAGGGGCTGGACACCCAGCGGTTCTCGGG
CAAGAGGTTGGGGGACACCTTCGTCCTCACCCTGAGCGACTTCCGCCGAGAGAACGAGGGCTACTATT
TCTGCTCGGCCCTGAGCAACTCCATCATGTACTTCAGCCACTTCGTGCCGGTCTTCCTGCCAGCGAAG
CCCACCACGACGCCAGCGCCGCGACCACCAACACCGGCGCCCACCATCGCGTCGCAGCCCCTGTCCCT
GCGCCCAGAGGCGTGCCGGCCAGCGGCGGGGGGCGCAGTGCACACGAGGGGGCTGGACTTCGCCTGTG
ATATCTACATCTGGGCGCCCTTGGCCGGGACTTGTGGGGTCCTTCTCCTGTCACTGGTTATCACCCTT
TACTGCAACCACAGGAACCGAAGACGTGTTTGCAAATGTCCCCGGCCTGTGGTCAAATCGGGAGACAA
GCCCAGCCTTTCGGCGAGATACGTCGGTTCAAGAGCTAAAAGAAGTGGTAGTGGTGCCCCTGTGA
SEQ ID NO: 5; (MAGE A4 TCR α chain) CDRs bold underlined
MKKHLTTFLVILWLYFYRGNGKNQVEQSPQSLIILEGKNCTLQCNYTVSPFSNLRWYKQDTGRGPVSL
TILTFSENTKSNGRYTATLDADTKQSSLHITASQLSDSASYICVVSGGTDSWGKLQFGAGTQVVVTPD
IQNPDPAVYQLRDSKSSDKSVCLFTDFDSQTNVSQSKDSDVYITDKTVLDMRSMDFKSNSAVAWSNKS
DFACANAFNNSIIPEDTFFPSPESSCDVKLVEKSFETDINLNFQNLSVIGFRILLLKVAGENLLMTLR
LWSSGSRAKR
SEQ ID NO: 6; (MAGE A4 TCR α chain coding sequence)
ATGAAGAAGCACCTGACCACCTTTCTCGTGATCCTGTGGCTGTACTTCTACCGGGGCAACGGCAAGAA
CCAGGTGGAACAGAGCCCCCAGAGCCTGATCATCCTGGAAGGCAAGAACTGCACCCTGCAGTGCAACT
ACACCGTGTCCCCCTTCAGCAACCTGCGGTGGTACAAGCAGGACACCGGCAGAGGCCCTGTGTCCCTG
ACCATCCTGACCTTCAGCGAGAACACCAAGAGCAACGGCCGGTACACCGCCACCCTGGACGCCGATAC
AAAGCAGAGCAGCCTGCACATCACCGCCAGCCAGCTGAGCGATAGCGCCAGCTACATCTGCGTGGTGT
CCGGCGGCACAGACAGCTGGGGCAAGCTGCAGTTTGGCGCCGGAACACAGGTGGTCGTGACCCCCGAC
ATCCAGAACCCTGACCCTGCCGTGTACCAGCTGCGGGACAGCAAGAGCAGCGACAAGAGCGTGTGCCT
GTTCACCGACTTCGACAGCCAGACCAACGTGTCCCAGAGCAAGGACAGCGACGTGTACATCACCGACA
AGACCGTGCTGGACATGCGGAGCATGGACTTCAAGAGCAATAGCGCCGTGGCCTGGTCCAACAAGAGC
GACTTCGCCTGCGCCAACGCCTTCAACAACAGCATTATCCCCGAGGACACATTCTTCCCAAGCCCCGA
GAGCAGCTGCGACGTCAAGCTGGTGGAAAAGAGCTTCGAGACAGACACCAACCTGAACTTCCAGAACC
TGAGCGTGATCGGCTTCAGAATCCTGCTGCTGAAGGTGGCCGGCTTCAACCTGCTGATGACCCTGAGA
CTGTGGTCCAGCGGCAGCCGGGCCAAGAGA
SEQ ID NO: 7; (MAGE A4 TCR β chain) CDRs bold underlined
MASLLFFCGAFYLLGTGSMDADVTQTPRNRITKTGKRIMLECSQTKGHDRMYWYRQDPGLGLRLIYYS
FDVKDINKGEISDGYSVSRQAQAKFSLSLESAIPNQTALYFCATSGQGAYEEQFFGPGTRLTVLEDLK
NVFPPEVAVFEPSEAEISHTQKATLVCLATGFYPDHVELSWWVNGKEVHSGVSTDPQPLKEQPALNDS
RYCLSSRLRVSATFWQNPRNHERCQVQFYGLSENDEWTQDRAKPVTQIVSAEAWGRADCGFTSESYQQ
GVLSATILYEILLGKATLYAVLVSALVLMAMVKRKDSRG
SEQ ID NO: 8; (MAGE A4 TCR β chain coding sequence)
ATGGCCAGCCTGCTGTTCTTCTGCGGCGCCTTCTACCTGCTGGGCACCGGCTCTATGGATGCCGACGT
GACCCAGACCCCCCGGAACAGAATCACCAAGACCGGCAAGCGGATCATGCTGGAATGCTCCCAGACCA
AGGGCCACGACCGGATGTACTGGTACAGACAGGACCCTGGCCTGGGCCTGCGGCTGATCTACTACAGC
TTCGACGTGAAGGACATCAACAAGGGCGAGATCAGCGACGGCTACAGCGTGTCCAGACAGGCTCAGGC
CAAGITCAGCCTGTCCCTGGAAAGCGCCATCCCCAACCAGACCGCCCTGTACTTTTGTGCCACAAGCG
GCCAGGGCGCCTACGAGGAGCAGTTCTTTGGCCCTGGCACCCGGCTGACAGTGCTGGAAGATCTGAAG
AACGTGTTCCCCCCAGAGGTGGCCGTGTTCGAGCCTTCTGAGGCCGAAATCAGCCACACCCAGAAAGC
CACACTCGTGTGTCTGGCCACCGGCTTCTACCCCGACCACGTGGAACTGTCTTGGTGGGTCAACGGCA
AAGAGGTGCACAGCGGCGTGTCCACCGATCCCCAGCCTCTGAAAGAACAGCCCGCCCTGAACGACAGC
CGGTACTGCCTGAGCAGCAGACTGAGAGTGTCCGCCACCTTCTGGCAGAACCCCAGAAACCACTTCAG
ATGCCAGGTGCAGTTTTACGGCCTGAGCGAGAACGACGAGTGGACCCAGGACAGAGCCAAGCCCGTGA
CACAGATCGTGTCTGCCGAAGCTTGGGGGCGCGCCGATTGTGGCTTTACCAGCGAGAGCTACCAGCAG
GGCGTGCTGAGCGCCACCATCCTGTACGAGATCCTGCTGGGAAAGGCCACACTGTACGCCGTGCTGGT
GTCTGCCCTGGTGCTGATGGCCATGGTCAAGCGGAAGGACAGCCGGGGC
SEQ ID NO: 9; (MAGE A4 TCR a chain variable region) 136AA-CDRs bold
underlined
MKKHLTTFLVILWLYFYRGNGKNQVEQSPQSLIILEGKNCTLQCNYTVSPFSNLRWYKQDTGRGPVSL
TILTFSENTKSNGRYTATLDADTKQSSLHITASQLSDSASYICVVSGGTDSWGKLQFGAGTQVVVTPD
SEQ ID NO: 10; (MAGE A4 TCR β chain variable region) 133AA-CDRs
bold underlined
MASLLFFCGAFYLLGTGSMDADVTQTPRNRITKTGKRIMLECSQTKGHDRMYWYRQDPGLGLRLIYYS
FDVKDINKGEISDGYSVSRQAQAKFSLSLESAIPNQTALYFCATSGOGAYEEQFFGPGTRLTVLE
SEQ ID NO: 11; CDR1 MAGE A4 TCR α chain, (residues 48-53)
VSPFSN
SEQ ID NO: 12; CDR2 MAGE A4 TCR α chain, (residues 71-76)
LTFSEN
SEQ ID NO: 13; CDR3 MAGE A4 TCR α chain, (residues 111-125)
CVVSGGTDSWGKLQF
SEQ ID NO: 14; CDR1 MAGE A4 TCR β chain, (residues 46-50)
KGHDR
SEQ ID NO: 15; CDR2 MAGE A4 TCR β chain, (residues 68-73)
SFDVKD
SEQ ID NO: 16; CDR3 MAGE A4 TCR β chain, (residues 110-123)
CATSGQGAYEEQFF
SEQ ID NO: 17; CDR1 CD8α (residues 45-53)
VLLSNPTSG
SEQ ID NO: 18; CDR2 CD8α (residues 72-79)
YLSQNKPK
SEQ ID NO: 19; CDR3 CD8α (residues 118-123)
LSNSIM
SEQ ID NO: 20 Human Alpha-fetoprotein
MKWVESIFLI FLLNFTESRT LHRNEYGIAS ILDSYQCTAE ISLADLATIF
FAQFVQEATY KEVSKMVKDA LTAIEKPTGD EQSSGCLENQ LPAFLEELCH
EKEILEKYGH SDCCSQSEEG RHNCFLAHKK PTPASIPLFQ VPEPVTSCEA
YEEDRETFMN KFIYEIARRH PFLYAPTILL WAARYDKIIP SCCKAENAVE
CFQTKAATVT KELRESSLLN QHACAVMKNF GTRIFQAITV TKLSQKFTKV
NFTEIQKLVL DVAHVHEHCC RGDVLDCLOD GEKIMSYICS QQDTLSNKIT
ECCKLTTLER GQCIIHAEND EKPEGLSPNL NRFLGDRDEN QFSSGEKNIE
LASFVHEYSR RHPQLAVSVI LRVAKGYQEL LEKCFQTENP LECQDKGEEE
LOKYIQESQA LAKRSCGLFQ KLGEYYLQNA FLVAYTKKAP QLTSSELMAI
TRKMAATAAT CCQLSEDKLL ACGEGAADII IGHLCIRHEM TPVNPGVGQC
CTSSYANRRP CFSSLVVDET YVPPAFSDDK FIFHKDLCQA QGVALQTMKQ
EFLINLVKQK PqITEEQLEA VIADFSGLLE KCCOGQEQEV CFAEEGQKLI
SKTRAALGV
SEQ ID NO: 21 Human Alpha-fetoprotein peptide
FMNKFIYEI
Parental AFP TCR TRAV12-2*02/TRAJ41*01/TRAC alpha chain amino acid
extracellular sequence (SEQ ID No: 22)
                                    10 20
                                    * *
Q K E V E Q N S G P L S V P E G A I A S L N C T Y S D
        30 40 50
        * * *
R G S Q S F F W Y R Q Y S G K S P E L I M S I Y S N G
                    60 70 80
                    * * *
D K E D G R E T A Q L N K A S Q Y V S L L I R D S Q P
                                90 100
                                * *
S D S A T Y L C A V N S D S G Y A L N F G K G T S L L
  110 120 130
    * * *
V I P H I Q N P D P A V Y Q L R D S K S S D K S V C L
              140 150 160
                * * *
F T D F D S Q T N V S Q S K D S D V Y I T D K T V L D
                          170 180
                            * *
M R S M D F K S N S A V A W S N K S D F A C A N A F N
190 200
* *
N S I I P E D T F F P S P E S S
Parental AFP TCR TRBV9*01/TRBD2/TRBJ2-7*01/TRBC2 beta chain amino
acid extracellular sequence (SEQ ID No: 23)
                                    10 20
                                    * *
D S G V T Q T P K H L I T A T G Q R V t L R C S P R S
        30 40 50
        * * *
G D L S V Y W Y Q Q S L D Q G L Q F L I Q Y Y N G E E
                    60 70 80
                    * * *
R A K G N I L E R F S A Q Q F P D L H S E L N L S S L
                                90 100
                                * *
E L G D S A L Y F C A S S L G G E S E Q Y F G P G T R
  110 120 130
    * * *
L T V T E D L K N V E P P E V A V F E P S E A E I S H
              140 150 160
                * * *
T Q K A T L V C L A T G F Y P D H V E L S W W V N G K
                          170 180
                            * *
E V H S G V S T D P Q P L K E Q P A L N D S R Y C L S
190 200 210
* * *
S R L R V S A T F W Q N P R N H F R C Q V Q F Y G L S
          220 230 240
            * * *
                E N D E W T Q D R A K P V T Q I V S A E A W G R A D
Reference TCR alpha chain DNA sequence (SEQ ID No: 24)
caaaaagaagttgagcagaattctggacccctcagtgttccagagggagccattgcctctctcaactg
cacttacagtgaccgaggttcccagtccttcttctggtacagacaatattctgggaaaagccctgagt
tgataatgtccatatactccaatggtgacaaagaagatggaaggtttacagcacagctcaataaagcc
agccagtatgtttctctgctcatcagagactcccagcccagtgattcagccacctacctctgtgccgt
gaatagtgattccgggtatgcactcaacttcggcaaaggcacctcgctgttggtcacaccccatatcc
agaaccctgaccctgccgtgtaccagctgagagactctaagtcgagtgacaagtctgtctgcctattc
accgattttgattctcaaacaaatgtgtcacaaagtaaggattctgatgtgtatatcacagacaaatg
tgtgctagacatgaggtctatggacttcaagagcaacagtgctgtggcctggagcaacaaatctgact
ttgcatgtgcaaacgccttcaacaacagcattattccagaagacaccttcttccccagcccagaaagt
tcc
Reference TCR beta chain DNA sequence (SEQ ID No: 25)
gattctggagtcacacaaaccccaaagcacctgatcacagcaactggacagcgagtgacgctgagatg
ctcccctaggtctggagacctctctgtgtactggtaccaacagagcctggaccagggcctccagttcc
tcattcagtattataatggagaagagagagcaaaaggaaacattcttgaacgattctccgcacaacag
ttccctgacttgcactctgaactaaacctgagctctctggagctgggggactcagctttgtatttctg
tgccagcagcctcgggggggaatctgagcagtacttcgggccgggcaccaggctcacggtcacagagg
acctgaaaaacgtgttcccacccgaggtcgctgtgtttgagccatcagaagcagagatctcccacacc
caaaaggccacactggtgtgcctggccaccggtttctaccccgaccacgtggagctgagctggtgggt
gaatgggaaggaggtgcacagtggggtctgcacagacccgcagcccctcaaggagcagcccgccctca
atgactccagatacgctctgagcagccgcctgagggtctcggccaccttctggcaggacccccgcaac
cacttccgctgtcaagtccagttctacgggctctcggagaatgacgagtggacccaggatagggccaa
acccgtcacccagatcgtcagcgccgaggcctggggtagagcagac
DRGSQS (αCDR1), SEQ ID NO:26
IYSNGD (αCDR2) , SEQ ID NO: 27
AVNSDSGYALNF (αCDR3) , SEQ ID NO: 28
SGDLS (βCDR1), SEQ ID NO: 29
YYNGEE (βCDR2) , SEQ ID NO: 30
ASSLGGESEQY (βCDR3) , SEQ ID NO: 31
DRGSQA (αCDR1), SEQ ID NO: 32
AVNSDSSYALNF (αDR2), SEQ ID NO: 33
AVNSDSGVALNE (αCDR2), SEQ ID NO: 34
AVNSQSGYALNF (αCDR2), SEQ ID NO: 35
AVNSQSGYSLNE (αCDR2), SEQ ID NO: 36
AVNSQNGYALNF (αCDR2), SEQ ID NO: 37
DRGSFS (αCDR1), SEQ ID NO: 38
DRGSYS (αCDR1), SEQ ID NO: 39
AVNSQSSYALNF (xCDR2), SEQ ID NO: 40
Variant AFP TCR (AFP TRAV12-2*02/TRAJ41*01/TRAC alpha chain amino
acid extracellular sequence, CDRs underlined (SEQ ID No: 41)
QKEVEQNSGPLSVPEGAIASLNCTYSDRGSQAFFWYRQYSGKSPELIMSIYSNGDKEDGRETAQLNKA
SQYVSLLIRDSQPSDSATYLCAVNSQSGYALNFGKGTSLLVTPHIQNPDPAVYQLRDSKSSDKSVCLF
TDFDSQTNVSQSKDSDVYITDKTVLDMRSMDFKSNSAVAWSNKSDFACANAFNNSIIPEDTFFPSPES
S
Variant AFP TCR TRBV9*01/TRBD2/TRBJ2-7*01/TRBC2 beta chain amino
acid extracellular sequence, CDRs underlined (SEQ ID No: 42)
DSGVTQTPKHLITATGQRVTLRCSPRSGDLSVYWYQQSLDQGLOFLIQYYNGEERAKGNILERFSAQQ
FPDLHSELNLSSLELGDSALYFCASSLGGESEQYFGPGTRLTVTEDLKNVFPPEVAVFEPSEAEISHT
QKATLVCLATGFYPDHVELSWWVNGKEVHSGVSTDPQPLKEQPALNDSRYCLSSRLRVSATFWONPRN
HFRCQVQFYGLSENDEWTQDRAKPVTQIVSAEAWGRAD
Left Homology Arm (LHA), SEQ ID NO: 43
TTTGGGGTTGCTCCAAGGTAAAGTTCAAAAAGTATCCTGCAGTCAACCCTTTAGCACCATAAAGAAAC
TAAATTATTTAGATGTTTTTATGAGAACATATCAAAAAGTACTTTTCTGTCATCCAATACTTCCACAA
ATAAATCATTAGTTCTTGCTAATCTTCATCTGGCATAAAAATAATGACATCAACTTTCTTCATGTAAT
TTCCCACTTAATTCCTTTACTAGGAGCAATATCAATTCCTATATGACGTCATTGCCAGCACCIACCCT
GCTCAGAATGGACAAGTAAAGAAAAACAACCATCAAGAAGATAAAATTGAATTTGATAATGAAGTGGA
CAAAGTAAAGCAGGATGCTAATTGTGTTAATCCACTTGGTGCCCCAGAAAAGCTCCCTGAAGCAAAGG
AACAGGCTGAAGGTTCTGAACCCACGAGTGGCACTGAGGGGCCAGAACATTCTGTCAATGGTCCTGCA
AGTCCAGCTTTAAATCAAGGTTCA
Right Homology Arm (RHA), SEQ ID NO: 44
GAAAAGACATAAATGAGGAAACTCCAAACCTCCTGTTAGCTGTTATTTCTATTTTTGTAGAAGTAGGA
AGTGAAAATAGGTATACAGTGGATTAATTAAATGCAGCGAACCAATATTTGTAGAAGGGTTATATTTT
ACTACTGTGGAAAAATATTTAAGATAGTTTTGCCAGAACAGTTTGTACAGACGTATGCTTATTTTAAA
ATTTTATCTCTTATTCAGTAAAAAACAACTTCTTTGTAATCGTTATGTGTGTATATGTATGTGTGTAT
GGGTGTGTGTTTGTGTGAGAGACAGAGAAAGAGAGAGAATTCTTTCAAGTGAATCTAAAAGCTTTTGC
TTTTCCTTTGTTTTTATGAAGAAAAAATACATTTTATATTAGAAGTGTTAACTTAGCTTGAAGGATCT
GTTTTTAAAAATCATAAACTGTGTGCAGACTCAATAAAATCATGTACATTTCTGAAATGACCTCAAGA
TGTCCTCCTTGTTCTACTCATATA
FuT2A_A2M4, SEQ ID NO: 45
TCCAGCGGCAGCCGGGCCAAGAGATCTGGATCAGGTGAGGGCAGAGGCAGCCTGCTGACATGTGGCGA
CGTGGAAGAAAACCCTGGCCCTATGAAGAAGCACCTGACCACCTTTCTCGTGATCCTGTGGCTGTACT
TCTACCGGGGCAACGGCAAGAACCAGGTGGAACAGAGCCCCCAGAGCCTGATCATCCTGGAAGGCAAG
AACTGCACCCTGCAGTGCAACTACACCGTGTCCCCCTTCAGCAACCTGCGGTGGTACAAGCAGGACAC
CGGCAGAGGCCCTGTGTCCCTGACCATCCTGACCTTCAGCGAGAACACCAAGAGCAACGGCCGGTACA
CCGCCACCCTGGACGCCGATACAAAGCAGAGCAGCCTGCACATCACCGCCAGCCAGCTGAGCGATAGC
GCCAGCTACATCTGCGTGGTGTCCGGCGGCACAGACAGCTGGGGCAAGCTGCAGTTTGGCGCCGGAAC
ACAGGTGGTCGTGACCCCCGACATCCAGAACCCTGACCCTGCCGTGTACCAGCTGCGGGACAGCAAGA
GCAGCGACAAGAGCGTGTGCCTGTTCACCGACTTCGACAGCCAGACCAACGTGTCCCAGAGCAAGGAC
AGCGACGTGTACATCACCGACAAGACCGTGCTGGACATGCGGAGCATGGACTTCAAGAGCAATAGCGC
CGTGGCCTGGTCCAACAAGAGCGACTTCGCCTGCGCCAACGCCTTCAACAACAGCATTATCCCCGAGG
ACACATTCTTCCCAAGCCCCGAGAGCAGCTGCGACGTCAAGCTGGTGGAAAAGAGCTTCGAGACAGAC
ACCAACCTGAACTTCCAGAACCTGAGCGTGATCGGCTTCAGAATCCTGCTGCTGAAGGTGGCCGGCTT
CAACCTGCTGATGACCCTGAGACTGTGGTCCAGCGGCAGCCGGGCCAAGAGATCTGGATCCGGCGCTA
CCAACTTTAGCCTGCTGAAGCAGGCCGGGGACGTGGAAGAAAACCCTGGCCCTAGGATGGCCAGCCTG
CTGTTCTTCTGCGGCGCCTTCTACCTGCTGGGCACCGGCTCTATGGATGCCGACGTGACCCAGACCCC
CCGGAACAGAATCACCAAGACCGGCAAGCGGATCATGCTGGAATGCTCCCAGACCAAGGGCCACGACC
GGATGTACTGGTACAGACAGGACCCTGGCCTGGGCCTGCGGCTGATCTACTACAGCTTCGACGTGAAG
GACATCAACAAGGGCGAGATCAGCGACGGCTACAGCGTGTCCAGACAGGCTCAGGCCAAGTTCAGCCT
GTCCCTGGAAAGCGCCATCCCCAACCAGACCGCCCTGTACTTTTGTGCCACAAGCGGCCAGGGCGCCT
ACGAGGAGCAGTTCTTTGGCCCTGGCACCCGGCTGACAGTGCTGGAAGATCTGAAGAACGTGTTCCCC
CCAGAGGTGGCCGTGTTCGAGCCTTCTGAGGCCGAAATCAGCCACACCCAGAAAGCCACACTCGTGTG
TCTGGCCACCGGCTTCTACCCCGACCACGTGGAACTGTCTTGGTGGGTCAACGGCAAAGAGGTGCACA
GCGGCGTGTCCACCGATCCCCAGCCTCTGAAAGAACAGCCCGCCCTGAACGACAGCCGGTACTGCCTG
AGCAGCAGACTGAGAGTGTCCGCCACCTTCTGGCAGAACCCCAGAAACCACTTCAGATGCCAGGTGCA
GTTTTACGGCCTGAGCGAGAACGACGAGTGGACCCAGGACAGAGCCAAGCCCGTGACACAGATCGTGT
CTGCCGAAGCTTGGGGGCGCGCCGATTGTGGCTTTACCAGCGAGAGCTACCAGCAGGGCGTGCTGAGC
GCCACCATCCTGTACGAGATCCTGCTGGGAAAGGCCACACTGTACGCCGTGCTGGTGTCTGCCCTGGT
GCTGATGGCCATGGTCAAGCGGAAGGACAGCCGGGGCTAA
Synthetic PolyA sequence, SEQ ID NO : 46
AATAAAATATCTTTATTTTCATTACATCTGTGTGTTGGTTTTTTGTGTG
EF1A promoter, SEQ ID NO : 47
GCTCCGGTGCCCGTCAGTGGGCAGAGCGCACATCGCCCACAGTCCCCGAGAAGTTGGGGGGAGGGGTC
GGCAATTGAACCGGTGCCTAGAGAAGGTGGCGCGGGGTAAACTGGGAAAGTGATGTCGTGTACTGGCT
CCGCCTTTTTCCCGAGGGTGGGGGAGAACCGTATATAAGTGCAGTAGTCGCCGTGAACGTTCTTTTTC
GCAACGGGTTTGCCGCCAGAACACAG
LOXP, SEQ ID NO: 48
ATAACTTCGTATAATGTATGCTATACGAAGTTAT
NEO Resistance gene, SEQ ID NO : 49
ATGGGATCGGCCATTGAACAAGATGGATTGCACGCAGGTTCTCCGGCCGCTTGGGTGGAGAGGCTATT
CGGCTATGACTGGGCACAACAGACGATCGGCTGCTCTGATGCCGCCGTGTTCCGGCTGTCAGCGCAGG
GGCGCCCGGTTCTTTTTGTCAAGACCGACCTGTCCGGTGCCCTGAATGAACTGCAGGACGAGGCAGCG
CGGCTATCGTGGCTGGCCACGACGGGCGTTCCTTGCGCAGCTGTGCTCGACGTTGTCACTGAAGCGGG
AAGGGACTGGCTGCTATTGGGCGAAGTGCCGGGGCAGGATCTCCTGTCATCTCACCTTGCTCCTGCCG
AGAAAGTATCCATCATGGCTGATGCAATGCGGCGGCTGCATACGCTTGATCCGGCTACCTGCCCATTC
GACCACCAAGCGAAACATCGCATCGAGCGAGCACGTACTCGGATGGAAGCCGGTCTTGTCGATCAGGA
TGATCTGGACGAAGAGCATCAGGGGCTCGCGCCAGCCGAACTGTTCGCCAGGCTCAAGGCGCGCATGC
CCGACGGCGAGGATCTCGTCGTGACCCATGGCGATGCCTGCTTGCCGAATATCATGGTGGAAAATGGC
CGCTTTTCTGGATTCATCGACTGTGGCCGGCTGGGTGTGGCGGACCGCTATCAGGACATAGCGTTGGC
TACCCGTGATATTGCTGAAGAGCTTGGCGGCGAATGGGCTGACCGCTTCCTCGTGCTTTACGGTATCG
CCGCTCCCGATTCGCAGCGCATCGCCTTCTATCGCCTTCTTGACGAGTTCTTCTGA
Primer PTPRC-5int-FWD (SEQ ID NO: 50)
CAGTGATTCCTGCCCTGATTCTTA
Primer PTPRC-5int-REV (SEQ ID NO: 51)
TACAGCCACAGGATCACGAGAAAG
Primer PTPRC-3int-FWD (SEQ ID NO: 52)
CCTGCCGAGAAAGTATCCATCAT
Primer PTPRC-3int-REV (SEQ ID NO: 53).
TAGCATACACACACATACCACCTT
Primer ITR_F (SEQ ID NO: 54)
GGAACCCCTAGTGATGGAGTT
Primer ITR_R (SEQ ID NO: 55)
CGGCCTCAGTGAGCGA
```

1. A modified induced pluripotent stem cell iPSC or haemogenic lineage cell comprising at least one heterologous nucleic acid sequence encoding a heterologous T-cell receptor (TCR) integrated at or into a locus in the cell genome.

2. The modified induced pluripotent stem cell iPSC or haemogenic lineage cell according to claim 1, wherein the at least one heterologous nucleic acid sequence encoding a heterologous TCR is an expressible heterologous nucleic acid sequence.

3. The modified induced pluripotent stem cell iPSC or haemogenic lineage cell according to either claim 1 or claim 2, wherein the modified induced pluripotent stem cell iPSC or haemogenic lineage cell expresses or presents the at least one heterologous TCR encoded by a heterologous TCR encoding nucleic acid sequence, preferably expressed or presented at the cell surface.

4. The modified induced pluripotent stem cell iPSC or haemogenic lineage cell according to any previous claim, wherein the modified haemogenic lineage cell is derived from the modified iPSC.

5. The modified induced pluripotent stem cell iPSC or haemogenic lineage cell according to any previous claim, wherein the haemogenic lineage cell is selected from any one of;
(a) a mesoderm cell, optionally which may express any one or more of the mesodermal markers, Brachyury, Goosecoid, MixI1, KDR (also known as FLK1 or VEGFR2), FoxA2, GATA6 or PDGF alpha R,
(b) a haemogenic endothelial cell, optionally which may be (a) CD34+ or (b) CD34+CD73− or (c) CD34+CD73−CXCR4− (CD184−),
(c) a haematopoietic progenitor cell, optionally which may be (a) CD34+ or (b) CD34+CD45+ or (c) CD34+ and/or CD45+ in combination with any one or more of CD117+, CD133+, CD45+, FLK+, CD38−, (d) CD34+, CD133+, CD45+, FLK1+, CD38−
(d) a progenitor T cell, optionally which may be (a) CD5+ and/or CD7+, or (b) CD5+ and/or CD7+ in combination with any one or more of: CD44+, CD25+, CD2+, CD45+, CD3−, CD4−, CD8−,
(e) a double positive T cell (DP T cell), optionally which may be (a) CD4+CD8+(b) CD4+CD8+ in combination with any one or more of CD3+, CD28+, CD45+,
(f) a single positive T cells (SP T cell) optionally which may be (a) CD4+(b) CD8+(b) CD4+ or CD8+ in combination with any one or more of CD3+, CD28+, CD45+, or
(g) a mature T cell, optionally alpha beta T cell, gamma delta T cell, NKT cell, T helper cell (TH) which are CD4+, cytotoxic T cell (TC) which are CD8+.

6. The modified induced pluripotent stem cell iPSC or haemogenic lineage cell according to any previous claim, wherein RAG1 expression is reduced or eliminated.

7. The modified induced pluripotent stem cell iPSC or haemogenic lineage cell according to claim 6, wherein the rag1 gene is inactivated or knocked-out.

8. The modified induced pluripotent stem cell iPSC or haemogenic lineage cell according to any previous claim, wherein the heterologous nucleic acid sequence encoding a heterologous TCR is integrated at or into one or both alleles of the locus in the cell genome.

9. The modified induced pluripotent stem cell iPSC or haemogenic lineage cell according to claim 6, wherein the locus is a gene encoding an endogenous protein of the cell.

10. The modified induced pluripotent stem cell iPSC or haemogenic lineage cell according to claim 7, wherein the heterologous nucleic acid sequence encoding a heterologous TCR is integrated adjacent to or within the gene encoding the endogenous protein

11. The modified induced pluripotent stem cell iPSC or haemogenic lineage cell according to either of claim 9 or 10, wherein the heterologous nucleic acid sequence encoding a heterologous TCR is integrated within an intron or exon of the gene encoding the endogenous protein, optionally a 3′ exon.

12. The modified induced pluripotent stem cell iPSC or haemogenic lineage cell according to claim 9, wherein the heterologous nucleic acid sequence encoding a heterologous TCR is integrated within the 3′ exon before the TAG stop sequence of the gene or nucleic add sequence encoding the endogenous protein.

13. The modified induced pluripotent stem cell iPSC or haemogenic lineage cell according to any one of claims 7 to 10, wherein the integration of the heterologous nucleic acid sequence encoding a heterologous TCR is non-disruptive to the production of the endogenous protein.

14. The modified induced pluripotent stem cell iPSC or haemogenic lineage cell according to any one of claims 9 to 13, comprising a fusion sequence between the nucleic acid encoding the heterologous TCR and the nucleic acid encoding the endogenous gene, preferably a fusion gene or sequence or multicistronic fusion gene or sequence between the nucleic acid encoding the heterologous TCR and the nucleic acid encoding the endogenous protein.

15. The modified induced pluripotent stem cell iPSC or haemogenic lineage cell according to claim 14 wherein the nucleic acid encoding the heterologous TCR is connected to the nucleic acid encoding the endogenous protein by a nucleic acid sequence encoding a peptide comprising an enzymatic cleavage site and/or a nucleic acid sequence which mediates ribosome-skipping.

16. The modified induced pluripotent stem cell iPSC or haemogenic lineage cell according to either of claim 14 or 15, wherein the nucleic acid encoding the heterologous TCR comprises a coding sequence of a TCRα and TCRβ chain, optionally with an intervening nucleic acid sequence encoding a peptide comprising an enzymatic cleavage site and/or nucleic acid sequence which mediates ribosome-skipping.

17. The modified induced pluripotent stem cell iPSC or haemogenic lineage cell according to either of claim 15 or 16, wherein the nucleic acid sequence which mediates ribosome-skipping is a T2A or P2A skip sequence.

18. The modified induced pluripotent stem cell iPSC or haemogenic lineage cell according to any one of claims 15 to 17, wherein the nucleic acid sequence encoding a peptide comprising an enzymatic cleavage site encodes a furin cleavage site, preferably RAKR.

19. The modified induced pluripotent stem cell iPSC or haemogenic lineage cell according to any one of claims 9 to 18, wherein the transcription and expression of the heterologous TCR and the endogenous protein is from the same promoter.

20. The modified induced pluripotent stem cell iPSC or haemogenic lineage cell according to any one of claims 9 to 19, wherein the heterologous TCR is expressed as a fusion protein with the endogenous protein, optionally connected by a peptide comprising an enzymatic cleavage site, preferably a furin cleavage site, preferably RAKR and/or ribosome skip sequence, preferably T2A or P2A skip sequence.

21. The modified induced pluripotent stem cell iPSC or haemogenic lineage cell according to any one of claims 1 to 20, wherein the heterologous TCR is expressed and/or presented in the cell as a nascent heterologous TCR comprising a TCR alpha chain and TCR beta chain, preferably expressed and/or presented at the cell surface.

22. The modified induced pluripotent stem cell iPSC or haemogenic lineage cell according to any one of claims 9 to 21, wherein the endogenous protein is a protein that is trafficked to the cell surface, optionally via the secretory pathway and/or to the plasma membrane.

23. The modified induced pluripotent stem cell iPSC or haemogenic lineage cell according to any one of claims 3 to 22, wherein the endogenous protein is a membrane protein or transmembrane protein, optionally a receptor protein, preferably a receptor protein tyrosine phosphatase (PTP), preferably CD45 or protein tyrosine phosphatase receptor type C or PTPRC.

24. The modified induced pluripotent stem cell iPSC or haemogenic lineage cell according to any previous claim, wherein the locus is or is in a gene encoding a membrane protein or transmembrane protein, optionally a receptor protein, preferably a receptor protein tyrosine phosphatase (PTP), preferably CD45 or protein tyrosine phosphatase receptor type C or PTPRC.

25. The modified induced pluripotent stem cell iPSC or haemogenic lineage cell according to any previous claim, wherein the locus is or is in the PTPRC (CD45) gene on chromosome 1.

26. The modified induced pluripotent stem cell iPSC or haemogenic lineage cell according to claim 25, wherein the integration is at exon 33 of the PTPRC (CD45) gene, optionally before the TAG stop codon or immediately adjacent to and/or before the TAG stop codon.

27. The modified induced pluripotent stem cell iPSC or haemogenic lineage cell according to any one of claim 14 to claim 26, wherein the fusion gene or sequence or multicistronic fusion gene or sequence comprises the nucleic acid encoding the heterologous TCR and nucleic acid encoding PTPRC with an intervening nucleic acid sequence encoding a peptide comprising an enzymatic cleavage site, preferably a furin cleavage site, and nucleic acid sequence which mediates ribosome-skipping, preferably selected from a T2A or P2A skip sequence and wherein the nucleic acid sequence encoding the heterologous TCR comprises the coding sequence of a TCRα and TCR chain, with an intervening nucleic acid sequence encoding a peptide comprising an enzymatic cleavage site, preferably a furin cleavage site, and nucleic acid sequence which mediates ribosome-skipping, preferably selected from T2A or P2A skip sequence.

28. The modified induced pluripotent stem cell iPSC or haemogenic lineage cell according to any previous claim, wherein the heterologous TCR binds or specifically binds to an antigen or peptide antigen thereof selected from,
(a) a cancer and/or tumour antigen or peptide antigen thereof, or
(b) a cancer and/or tumour antigen or peptide antigen thereof associated with a cancerous condition and/or presented by tumour or cancer cell or tissue.

29. The modified induced pluripotent stem cell iPSC or haemogenic lineage cell according to claim 28 wherein the cancer and/or tumour antigen or peptide antigen thereof is selected from;
(a) a cancer-testis antigen,
(b) a MAGE antigen,
(c) MAGE A4 or peptide antigen thereof, optionally a peptide antigen comprising the sequence GVYDGREHTV (SEQ ID NO: 2), or
(d) AFP or peptide antigen thereof, optionally a peptide antigen comprising the sequence FMNKFIYEI (SEQ ID No: 21).

30. The modified induced pluripotent stem cell iPSC or haemogenic lineage cell according to either one of claim 28 or 29 wherein the cancer and/or tumour antigen or peptide antigen thereof is complexed with a peptide presenting molecule, optionally major histocompatibility complex (MHC) or human leukocyte antigen (HLA), optionally class I or class II, optionally HLA-A2 or HLA-A*02, or HLA-A*0201.

31. The modified induced pluripotent stem cell iPSC or haemogenic lineage cell according to any previous claim, wherein the heterologous TCR comprises a TCR having;
(a) an alpha chain variable domain comprising an amino acid sequence that has at least 80%, identity to SEQ ID NO:9 or the sequence of amino acid residues 1-136 of SEQ ID NO:6, and/or the beta chain variable domain comprising an amino acid sequence that has at least 80%, identity to SEQ ID NO:10 or the sequence of amino acid residues 1-133 of SEQ ID NO:7, or
(b) an alpha chain variable domain comprising an amino acid sequence that has at least 80%, identity to the sequence of amino acid residues 1-112 of SEQ ID NO:22, and/or the beta chain variable domain comprising an amino acid sequence that has at least 80%, identity to the sequence of amino acid residues 1-112 of SEQ ID NO:23.

32. The modified induced pluripotent stem cell iPSC or haemogenic lineage cell according to any previous claim, wherein the iPSC or haemogenic lineage cell further comprises a nucleic acid encoding and/or expresses or presents a heterologous co-receptor, optionally wherein the co-receptor is a CD8 co-receptor.

33. The modified induced pluripotent stem cell iPSC or haemogenic lineage cell according to claim 32, wherein the heterologous CD8 co-receptor is heterodimer or homodimer, a CD8αb heterodimer or a CD8αα homodimer.

34. The modified induced pluripotent stem cell iPSC or haemogenic lineage cell according to either one of claim 32 or 33, wherein the heterologous CD8 co-receptor comprises;
(a) a CDR 1 of at least 80% sequence identity to amino acid sequence VLLSNPTSG, SEQ ID NO:17, CDR 2 of at least 80% sequence identity to amino acid sequence YLSQNKPK SEQ ID NO:18 and CDR 3 of at least 80% sequence identity amino acid sequence LSNSIM SEQ ID NO:19, or
(b) an amino acid sequence having at least 80% sequence identity to amino acids number 22 to 235 of SEQ ID NO: 19.

35. The modified induced pluripotent stem cell iPSC or haemogenic lineage cell according to any previous claim, wherein the iPSC or haemogenic lineage cell further comprises a nucleic acid encoding and/or expresses or presents a heterologous co-stimulatory ligand; optionally 4-1BBL or CD80.

36. The modified induced pluripotent stem cell iPSC or haemogenic lineage cell according to any one of claims 1 to 35, wherein the binding of the iPSC or haemogenic lineage cell and/or heterologous TCR to the cancer and/or tumour antigen or peptide antigen thereof or a cancer and/or tumour cell or tissue expressing or presenting the cancer and/or tumour antigen or peptide antigen thereof according to any of claims 28 to 30 induces activation of the iPSC or haemogenic lineage cell, optionally as determined by any one or more of;
(a) up-regulation of T cell activation markers, for example either CD69 and/or CD25 on CD3+ cells,
(b) up-regulation of cytokine production, for example any one or more of IFN gamma, IL-2 or Granzyme B,
(c) induced cell cytotoxic activity in the presence of the antigen or antigen peptide, or
(d) ability to kill tumour cells presenting the antigen or antigen peptide.

37. A nucleic acid construct or vector comprising a nucleic acid region encoding the heterologous TCR according to any previous claim and at least one homology region comprising a nucleic acid region homologous to a nucleic acid region at the locus in the cell genome for integration of the nucleic acid region encoding the heterologous TCR.

38. The nucleic acid construct or vector according to claim 37, wherein the locus is or is in a gene encoding a membrane protein or transmembrane protein, optionally a receptor protein, preferably a receptor protein tyrosine phosphatase (PTP), preferably CD45 or protein tyrosine phosphatase receptor type C or PTPRC.

39. The nucleic acid construct or vector according to either claim 37 or 38, wherein the locus is or is in the PTPRC (CD45) gene on chromosome 1.

40. The nucleic acid construct or vector according to any one of claims 37 to 39, wherein the locus is at exon 33 of the PTPRC (CD45) gene, optionally before the TAG stop codon or immediately adjacent to and/or before the TAG stop codon.

41. The nucleic acid construct or vector according to any one of claims 37 to 40, wherein the nucleic acid encoding the heterologous TCR comprises a coding sequence of a TCRα and TCRβ chain, optionally with an intervening nucleic acid sequence encoding a peptide comprising an enzymatic cleavage site and/or nucleic acid sequence which mediates ribosome-skipping, preferably a furin cleavage site, preferably RAKR and/or ribosome skip sequence, preferably T2A or P2A skip sequence.

42. The nucleic acid construct or vector according to any one of claims 37 to 41, wherein the construct or vector comprises a left hand and a right hand homology region each homologous to a nucleic acid region at the locus in the cell genome for integration of the nucleic acid region encoding the heterologous TCR and which flank opposite sides of the integration site.

43. The nucleic acid construct or vector according to any one of claims 37 to 42, wherein the construct or vector further comprises any one or more of:
(a) a recombination target sequence, preferably loxP (locus of X-over P1) sequence,
(b) an expressible selection marker sequence, preferably an antibiotic resistance gene, and optionally a neomycin resistance gene, preferably constitutively expressed from a promoter for example from an EF1A promoter.

44. The nucleic acid construct or vector according to any one of claims 37 to 43, wherein the construct or vector comprises a nucleotide sequence encoding the heterologous TCR comprising the sequence SEQ ID No: 45, homology regions comprising the sequences SEQ ID No: 43 and SEQ ID No: 44, a recombination target sequence comprising SEQ ID No: 48 and an expressible selection marker sequence comprising SEQ ID No: 47 and SEQ ID No: 49.

45. A process of producing a modified induced pluripotent stem cell iPSC or haemogenic lineage cell according to any one of claims 1 to 36 comprising introducing the nucleic acid construct or vector according to any one of claims 37 to 44 into an unmodified induced pluripotent stem cell iPSC or haemogenic lineage cell, optionally as defined in claim 5, under conditions to permit integration of the nucleic acid sequence encoding a heterologous T-cell receptor (TCR) at or into a locus in the cell genome and optionally isolating the modified induced pluripotent stem cell iPSC or haemogenic lineage cell.

46. A pharmaceutical composition comprising the modified induced pluripotent stem cell iPSC or haemogenic lineage cell according to any one of claims 1 to 36 and a pharmaceutically acceptable carrier.

47. The modified induced pluripotent stem cell iPSC or haemogenic lineage cell according to any one of claims 1 to 36, or pharmaceutical composition of claim 46, for use in therapy and/or medicine.

48. The modified induced pluripotent stem cell iPSC or haemogenic lineage cell according to any one of claims 1 to 36, or pharmaceutical composition of claim 46, for use in treatment, prevention or delaying the progression of cancer and/or tumour in an individual or subject optionally wherein the treatment is cancer immunotherapy therapy and/or adoptive T cell therapy, optionally allogenic adoptive T cell therapy.

49. Use of the modified induced pluripotent stem cell iPSC or haemogenic lineage cell according to any one of claims 1 to 36, or pharmaceutical composition of claim 46, in the manufacture of a medicament for the treatment of cancer and/or tumour in an individual or subject, optionally wherein the treatment is cancer immunotherapy therapy and/or adoptive T cell therapy, optionally allogenic adoptive T cell therapy.

50. A method of treating, preventing or delaying the progression of cancer and/or tumour in an individual or subject comprising administering to the individual the modified induced pluripotent stem cell iPSC or haemogenic lineage cell according to any one of claims 1 to 34, or pharmaceutical composition of claim 44, optionally wherein the treatment is cancer immunotherapy therapy and/or adoptive T cell therapy, optionally allogenic adoptive T cell therapy.

51. The modified induced pluripotent stem cell iPSC or haemogenic lineage cell according to any one of claims 1 to 36, or pharmaceutical composition of claim 46, for use according to any of claims 47 to 49 or in the method of claim 50, wherein the cancer and/or tumour is a solid tumour.

52. The modified induced pluripotent stem cell iPSC or haemogenic lineage cell according to any one of claims 1 to 36, or pharmaceutical composition of claim 46, for use according to any of claims 47 to 49, or in the method of claim 50, wherein the modified induced pluripotent stem cell iPSC or haemogenic lineage cell, or pharmaceutical composition is for use or used in combination with one or more further therapeutic agent optionally administered or for administration separately, sequentially or simultaneously.

53. A kit comprising,
(a) the modified induced pluripotent stem cell iPSC or haemogenic lineage cell according to any one of claims 1 to 36, or pharmaceutical composition of claim 46, and a package insert comprising instructions for use thereof for treating, preventing or delaying the progression of cancer and/or tumour in an individual or subject, or
(b) the modified induced pluripotent stem cell iPSC or haemogenic lineage cell according to any one of claims 1 to 36, or pharmaceutical composition of claim 46, and a package insert comprising instructions for use thereof for treating, preventing or delaying the progression of cancer and/or tumour in an individual or subject, in combination with one or more further therapeutic agent optionally administered or for administration separately, sequentially or simultaneously.


SEQUENCE LISTING (70 sequences)

SEQ ID NO: 1 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 2 - protein, 10 aa, Homo sapiens
GVYDGREHTV

SEQ ID NO: 3 - protein, 235 aa, Artificial Sequence
  source: /note=“Description of Artificial Sequence Synthetic polypeptide”
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 4 - DNA, 745 bp, Artificial Sequence
  source: /note=“Description of Artificial Sequence Synthetic polynucleotide”
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcggttcaagagctaaaagaagtggtagtggtgcccctgtga

SEQ ID NO: 5 - protein, 282 aa, Artificial Sequence
  source: /note=“Description of Artificial Sequence Synthetic polypeptide”
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 6 - DNA, 846 bp, Artificial Sequence
  source: /note=“Description of Artificial Sequence Synthetic polynucleotide”
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagaga

SEQ ID NO: 7 - protein, 311 aa, Artificial Sequence
  source: /note=“Description of Artificial Sequence Synthetic polypeptide”
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 8 - DNA, 933 bp, Artificial Sequence
  source: /note=“Description of Artificial Sequence Synthetic polynucleotide”
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

SEQ ID NO: 9 - protein, 136 aa, Artificial Sequence
  source: /note=“Description of Artificial Sequence Synthetic polypeptide”
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPD

SEQ ID NO: 10 - protein, 133 aa, Artificial Sequence
  source: /note=“Description of Artificial Sequence Synthetic polypeptide”
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLE

SEQ ID NO: 11 - protein, 6 aa, Artificial Sequence
  source: /note=“Description of Artificial Sequence Synthetic peptide”
VSPFSN

SEQ ID NO: 12 - protein, 6 aa, Artificial Sequence
  source: /note=“Description of Artificial Sequence Synthetic peptide”
LTFSEN

SEQ ID NO: 13 - protein, 15 aa, Artificial Sequence
  source: /note=“Description of Artificial Sequence Synthetic peptide”
CVVSGGTDSWGKLQF

SEQ ID NO: 14 - protein, 5 aa, Artificial Sequence
  source: /note=“Description of Artificial Sequence Synthetic peptide”
KGHDR

SEQ ID NO: 15 - protein, 6 aa, Artificial Sequence
  source: /note=“Description of Artificial Sequence Synthetic peptide”
SFDVKD

SEQ ID NO: 16 - protein, 14 aa, Artificial Sequence
  source: /note=“Description of Artificial Sequence Synthetic peptide”
CATSGQGAYEEQFF

SEQ ID NO: 17 - protein, 9 aa, Artificial Sequence
  source: /note=“Description of Artificial Sequence Synthetic peptide”
VLLSNPTSG

SEQ ID NO: 18 - protein, 8 aa, Artificial Sequence
  source: /note=“Description of Artificial Sequence Synthetic peptide”
YLSQNKPK

SEQ ID NO: 19 - protein, 6 aa, Artificial Sequence
  source: /note=“Description of Artificial Sequence Synthetic peptide”
LSNSIM

SEQ ID NO: 20 - protein, 609 aa, Artificial Sequence
  source: /note=“Description of Artificial Sequence Synthetic polypeptide”
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 21 - protein, 9 aa, Homo sapiens
FMNKFIYEI

SEQ ID NO: 22 - protein, 205 aa, Artificial Sequence
  source: /note=“Description of Artificial Sequence Synthetic polypeptide”
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 23 - protein, 242 aa, Artificial Sequence
  source: /note=“Description of Artificial Sequence Synthetic polypeptide”
DSGVTQTPKHLITATGQRVTLRCSPRSGDLSVYWYQQSLDQGLQFLIQYYNGEERAKGNILERFSAQQFPDLHSELNLSSLELGDSALYFCASSLGGESEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 24 - DNA, 615 bp, Artificial Sequence
  source: /note=“Description of Artificial Sequence Synthetic polynucleotide”
caaaaagaagttgagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaatagtgattccgggtatgcactcaacttcggcaaaggcacctcgctgttggtcacaccccatatccagaaccctgaccctgccgtgtaccagctgagagactctaagtcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcc

SEQ ID NO: 25 - DNA, 726 bp, Artificial Sequence
  source: /note=“Description of Artificial Sequence Synthetic polynucleotide”
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcctcgggggggaatctgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagac

SEQ ID NO: 26 - protein, 6 aa, Artificial Sequence
  source: /note=“Description of Artificial Sequence Synthetic peptide”
DRGSQS

SEQ ID NO: 27 - protein, 6 aa, Artificial Sequence
  source: /note=“Description of Artificial Sequence Synthetic peptide”
IYSNGD

SEQ ID NO: 28 - protein, 12 aa, Artificial Sequence
  source: /note=“Description of Artificial Sequence Synthetic peptide”
AVNSDSGYALNF

SEQ ID NO: 29 - protein, 5 aa, Artificial Sequence
  source: /note=“Description of Artificial Sequence Synthetic peptide”
SGDLS

SEQ ID NO: 30 - protein, 6 aa, Artificial Sequence
  source: /note=“Description of Artificial Sequence Synthetic peptide”
YYNGEE

SEQ ID NO: 31 - protein, 11 aa, Artificial Sequence
  source: /note=“Description of Artificial Sequence Synthetic peptide”
ASSLGGESEQY

SEQ ID NO: 32 - protein, 6 aa, Artificial Sequence
  source: /note=“Description of Artificial Sequence Synthetic peptide”
DRGSQA

SEQ ID NO: 33 - protein, 12 aa, Artificial Sequence
  source: /note=“Description of Artificial Sequence Synthetic peptide”
AVNSDSSYALNF

SEQ ID NO: 34 - protein, 12 aa, Artificial Sequence
  source: /note=“Description of Artificial Sequence Synthetic peptide”
AVNSDSGVALNF

SEQ ID NO: 35 - protein, 12 aa, Artificial Sequence
  source: /note=“Description of Artificial Sequence Synthetic peptide”
AVNSQSGYALNF

SEQ ID NO: 36 - protein, 12 aa, Artificial Sequence
  source: /note=“Description of Artificial Sequence Synthetic peptide”
AVNSQSGYSLNF

SEQ ID NO: 37 - protein, 12 aa, Artificial Sequence
  source: /note=“Description of Artificial Sequence Synthetic peptide”
AVNSQNGYALNF

SEQ ID NO: 38 - protein, 6 aa, Artificial Sequence
  source: /note=“Description of Artificial Sequence Synthetic peptide”
DRGSFS

SEQ ID NO: 39 - protein, 6 aa, Artificial Sequence
  source: /note=“Description of Artificial Sequence Synthetic peptide”
DRGSYS

SEQ ID NO: 40 - protein, 12 aa, Artificial Sequence
  source: /note=“Description of Artificial Sequence Synthetic peptide”
AVNSQSSYALNF

SEQ ID NO: 41 - protein, 205 aa, Artificial Sequence
  source: /note=“Description of Artificial Sequence Synthetic polypeptide”
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 42 - protein, 242 aa, Artificial Sequence; identical to SEQ ID NO: 23
  source: /note=“Description of Artificial Sequence Synthetic polypeptide”

SEQ ID NO: 43 - DNA, 500 bp, Artificial Sequence
  source: /note=“Description of Artificial Sequence Synthetic polynucleotide”
tttggggttgctccaaggtaaagttcaaaaagtatcctgcagtcaaccctttagcaccataaagaaactaaattatttagatgtttttatgagaacatatcaaaaagtacttttctgtcatccaatacttccacaaataaatcattagttcttgctaatcttcatctggcataaaaataatgacatcaactttcttcatgtaatttcccacttaattcctttactaggagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttca

SEQ ID NO: 44 - DNA, 500 bp, Artificial Sequence
  source: /note=“Description of Artificial Sequence Synthetic polynucleotide”
gaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatata

SEQ ID NO: 45 - DNA, 1944 bp, Artificial Sequence
  source: /note=“Description of Artificial Sequence Synthetic polynucleotide”
tccagcggcagccgggccaagagatctggatcaggtgagggcagaggcagcctgctgacatgtggcgacgtggaagaaaaccctggccctatgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagagatctggatccggcgctaccaactttagcctgctgaagcaggccggggacgtggaagaaaaccctggccctaggatggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggctaa

SEQ ID NO: 46 - DNA, 49 bp, Artificial Sequence
  source: /note=“Description of Artificial Sequence Synthetic oligonucleotide”
aataaaatatctttattttcattacatctgtgtgttggttttttgtgtg

SEQ ID NO: 47 - DNA, 230 bp, Artificial Sequence
  source: /note=“Description of Artificial Sequence Synthetic polynucleotide”
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 48 - DNA, 34 bp, Artificial Sequence
  source: /note=“Description of Artificial Sequence Synthetic oligonucleotide”
ataacttcgtataatgtatgctatacgaagttat

SEQ ID NO: 49 - DNA, 804 bp, Artificial Sequence
  source: /note=“Description of Artificial Sequence Synthetic polynucleotide”
atgggatcggccattgaacaagatggattgcacgcaggttctccggccgcttgggtggagaggctattcggctatgactgggcacaacagacgatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 50 - DNA, 24 bp, Artificial Sequence
  source: /note=“Description of Artificial Sequence Synthetic primer”
cagtgattcctgccctgattctta

SEQ ID NO: 51 - DNA, 24 bp, Artificial Sequence
  source: /note=“Description of Artificial Sequence Synthetic primer”
tacagccacaggatcacgagaaag

SEQ ID NO: 52 - DNA, 23 bp, Artificial Sequence
  source: /note=“Description of Artificial Sequence Synthetic primer”
cctgccgagaaagtatccatcat

SEQ ID NO: 53 - DNA, 24 bp, Artificial Sequence
  source: /note=“Description of Artificial Sequence Synthetic primer”
tagcatacacacacataccacctt

SEQ ID NO: 54 - DNA, 21 bp, Artificial Sequence
  source: /note=“Description of Artificial Sequence Synthetic primer”
ggaacccctagtgatggagtt

SEQ ID NO: 55 - DNA, 16 bp, Artificial Sequence
  source: /note=“Description of Artificial Sequence Synthetic primer”
cggcctcagtgagcga

SEQ ID NO: 56 - protein, 12 aa, Artificial Sequence
  source: /note=“Description of Artificial Sequence Synthetic peptide”
AVNSQSGVALNF

SEQ ID NO: 57 - DNA, 12 bp, Artificial Sequence
  source: /note=“Description of Artificial Sequence Synthetic probe”
tctgcgcgctcg

SEQ ID NO: 58 - DNA, 20 bp, Artificial Sequence
  source: /note=“Description of Artificial Sequence Synthetic oligonucleotide”
tcttttcaaaggatctcacc

SEQ ID NO: 59 - DNA, 20 bp, Artificial Sequence
  source: /note=“Description of Artificial Sequence Synthetic oligonucleotide”
gcaagtccagctttaaatca

SEQ ID NO: 60 - DNA, 20 bp, Artificial Sequence
  source: /note=“Description of Artificial Sequence Synthetic primer”
cttgggactcagttctgccc

SEQ ID NO: 61 - DNA, 20 bp, Artificial Sequence
  source: /note=“Description of Artificial Sequence Synthetic primer”
gaactcagtggggtggatcg

SEQ ID NO: 62 - DNA, 108 bp, Artificial Sequence
  source: /note=“Description of Artificial Sequence Synthetic polynucleotide”
  CDS (1)..(93)
gaacaggctgaaggttctgaacccacgagtggcactgaggggccagaacattctgtcaatggtcctgcaagtccagctttaaatcaaggttcataggaaaagacataa

SEQ ID NO: 63 - protein, 31 aa, Artificial Sequence
  source: /note=“Description of Artificial Sequence Synthetic polypeptide”
EQAEGSEPTSGTEGPEHSVNGPASPALNQGS

SEQ ID NO: 64 - DNA, 159 bp, Artificial Sequence
  source: /note=“Description of Artificial Sequence Synthetic polynucleotide”
  CDS (1)..(159)
tctgtcaatggtcctgcaagtccagctttaaatcaaggttcatccagcggcagccgggccaagagatctggatcaggtgagggcagaggcagcctgctgacatgtggcgacgtggaagaaaaccctggccctatgaagaagcacctgaccacctttctc

SEQ ID NO: 65 - protein, 53 aa, Artificial Sequence
  source: /note=“Description of Artificial Sequence Synthetic polypeptide”
SVNGPASPALNQGSSSGSRAKRSGSGEGRGSLLTCGDVEENPGPMKKHLTTFL

SEQ ID NO: 66 - DNA, 62 bp, Homo sapiens
cacatattaaattttcagaatggaaatttaagctgttccgggtgagatcctttgaaaagaca

SEQ ID NO: 67 - DNA, 51 bp, Artificial Sequence
  source: /note=“Description of Artificial Sequence Synthetic oligonucleotide”
cacatattaaattttcagaatggaaatttaagctgatcctttgaaaagaca

SEQ ID NO: 68 - DNA, 57 bp, Homo sapiens
atggaaatttaagctgttccgggtgagatcctttgaaaagacacctgaagaagctca

SEQ ID NO: 69 - DNA, 41 bp, Artificial Sequence
  source: /note=“Description of Artificial Sequence Synthetic oligonucleotide”
atggaaatttaatcctttgaaaagacacctgaagaagctca

SEQ ID NO: 70 - protein, 4 aa, Unknown
  source: /note=“Description of Unknown Furin cleavage site sequence”
RAKR